(19) United States
(12) Patent Application Publication
(10) Pub. No.: US 2015/0038354 A1
(43) Pub. Date: Feb. 5, 2015

(54) FULL LENGTH ANTIBODY DISPLAY SYSTEM FOR EUKARYOTIC CELLS AND ITS USE

(71) Applicant: Hoffmann-La Roche Inc., Nutley, NJ (US)

(72) Inventors: PETER MICHAEL HUELSMANN, HABACH (DE); HENDRIK KNOETGEN, PENZBERG (DE)

(73) Assignee: HOFFMANN-LA ROCHE INC., Nutley, NJ (US)

(21) Appl. No.: 14/310,309

(22) Filed: Jun. 20, 2014

Related U.S. Application Data
(63) Continuation of application No. PCT/EP2012/076163, filed on Dec. 19, 2012.

(30) Foreign Application Priority Data
Dec. 22, 2011 (EP) 11195375.8
Aug. 2, 2012 (EP) 12179029.9

(51) Int. Cl. G01N 33/577

(52) U.S. Cl. CPC G01N 33/577; C07K 2317/14
USPC 506/9; 435/320.1; 506/14

(57) ABSTRACT

Herein is reported a method of selecting a cell expressing a bispecific antibody comprising the steps of (a) generating a population of eukaryotic cells by transduction with a population of lentiviral virus particles, whereby each cell of the population of cells displays a membrane-bound full length antibody which is encoded by the lentiviral nucleic acid, and which specifically binds to two or more antigens or two or more epitopes on the same antigen, and (b) selecting from the population of eukaryotic cells a cell depending on the properties of the displayed membrane-bound full length antibody, whereby each lentiviral virus particle of the population of lentiviral virus particles comprises a bicistronic expression cassette comprising the EV71-IRES for the expression of the membrane-bound antibody.

### CROSS REFERENCE TO RELATED APPLICATIONS

This application is a continuation of International Application No. PCT/EP2012/076163 having an international filing date of Dec. 19, 2012, the entire contents of which are incorporated herein by reference, and which claims benefit under 35 U.S.C. §119 to European Patent Application No. 12179029.9, filed Aug. 2, 2012, and European Patent Application No. 11195375.8, filed Dec. 22, 2011.

### Sequence Listing

The instant application contains a Sequence Listing submitted via EFS-Web. Said ASCII copy, created on Jun. 17, 2014, is named P4837C1SeqList.txt, and is 11,484 bytes in size.

The present invention is related to the field of monoclonal antibodies, especially to nucleic acids encoding such antibodies. The invention provides methods for generating and selecting a eukaryotic cell expressing and displaying on its surface an antibody, especially a full length monoclonal antibody, especially a bispecific monoclonal antibody, which is capable of specifically binding one or more antigen(s) of interest.

### BACKGROUND OF THE INVENTION

Known methods for the isolation of recombinant antibodies are phage display (Hogenboom, Methods Mol. Biol. 178 (2002) 1-37), ribosome/mRNA display (Lipovsek and Plueckthun, J. Immunol. Method 290 (2004) 51-67) and microbial cell display (Boder and Wittrup, Nat. Biotechnol. 15 (1997) 553-557).

A screening system based on Vaccinia virus-mediated expression of whole antibodies in mammalian cells is reported in US 2002/0123057. Another screening system is based on cell surface expression of antibodies in mammalian cells (Ho, et al., Proc. Natl. Acad. Sci. USA 103 (2006) 9637-9642).

Phage display allows for the screening of 10¹² to 10¹³ clones in a single panning round (Barbas III, et al., (eds.), Phage Display—A Laboratory manual, Cold Spring Habour Press, (2001)), whereas the throughput of a mammalian screening procedure in a one antibody per cell formant is limited to the concomitant analysis of about 10⁶ to 10⁷ clones.

Cellular display is described by Higuchi et al. in COS cells (J. Immunol. Meth. 202 (1997) 193-204). Beerli et al. report a Sindbis virus-based scFv cell surface display library produced from antigen-specific B-cells in BHK cells (Proc. Natl. Acad. Sci. USA 105 (2008) 14336-14341). Ho and Pastan report methods with HEK293 cells (scFv) (Methods Mol. Biol. 562 (2009) 99-113). Lymphocyte display is reported by Alonso-Camino et al. (PLoS One. 4 (2009) e7174). Zhou et al. report methods using HEK293 cells (Acta Biochim. Biophys. Sin. 42 (2010) 575-584). Zhou et al. report the Flp-In system (MAbs. 2 (2010) 508-518).

Taube, R., et al report (PLOS One 3 (2008) e3181) stable expression of human antibodies on the surface of human cells and lentiviral virus particles.

In WO 2007/047578 the cell display of antibody libraries is reported.

### SUMMARY OF THE INVENTION

It has been found that full length antibodies can be expressed and displayed on eukaryotic cells by using a lentiviral virus particle comprising a bicistronic expression cassette. The expression of full length antibodies on eukaryotic cells using a lentiviral virus particle as reported herein is possible by using the IRES (internal ribosomal entry site) element of the EV71, which is linking either the expression cassettes of an antibody light chain and an antibody heavy chain or the expression cassettes of two antibody heavy chains.

In case of the presence of an antibody light chain expression cassette and an antibody heavy chain expression cassette in the lentiviral vector the antibody heavy chain expression cassette may comprises after the exon encoding the C-terminal antibody domain a non-constitutive splice site providing a means for expressing beside the soluble antibody heavy chain also a membrane-bound antibody heavy chain resulting in the presentation of membrane-bound full length antibody.

In case of the presence of two antibody heavy chain expression cassettes in the lentiviral vector either both or only the second antibody heavy chain expression cassette may comprises after the exon encoding the C-terminal antibody domain an exon encoding a transmembrane domain resulting in the presentation of membrane-bound full length antibody.

Further are provided herein methods for generating and selecting a eukaryotic cell expressing and displaying on its surface an antibody, especially a full length monoclonal antibody.

One aspect as reported herein is a lentiviral vector comprising a bicistronic expression cassette, which comprises in 5′- to 3′-direction
a promoter,
a first nucleic acid encoding a full length antibody light chain,
the EV71-IRES,
a second nucleic acid encoding a full length antibody heavy chain,
a spliceable intron, and
a transmembrane domain or a GPI-anchor.

This lentiviral vector as reported herein is an expression vector in that it comprises a bicistronic expression cassette for the expression of a full length antibody light chain and a full length antibody heavy chain, wherein the IRES separating the two expression cassettes is the EV71-IRES.

It has been found that only the EV71-IRES can be used for the expression of full length antibody in a bicistronic expression cassette in a lentiviral expression system.

Due to the provision of the spliceable intron a soluble form of the antibody heavy chain as well as a membrane-bound form of the antibody heavy chain can be expressed from the expression vector as reported herein.

By expression of a soluble form and a membrane-bound form of the antibody heavy chain the cell on the one hand secretes a full length antibody, which can be tested e.g. in an ELISA, and at the same time presents a membrane-bound form of the full length antibody on its surface, which can be used for the selection of the cell e.g. by FACS enabling the isolation of a single cell clone.

One aspect as reported herein is a lentiviral vector comprising a bicistronic expression cassette, which comprises in 5′- to 3′-direction
a promoter,
a first nucleic acid encoding a first full length antibody heavy chain,
the EV71-IRES,
a second nucleic acid encoding a second full length antibody heavy chain, and
a transmembrane domain or a GPI-anchor.

This lentiviral vector as reported herein is an expression vector in that it comprises a bicistronic expression cassette for the expression of two different full length antibody heavy chains, wherein the IRES separating the two expression cassettes is the EV71-IRES.

By expression of a membrane-bound form of the antibody heavy chain the cell presents a membrane-bound form of the full length antibody on its surface, which can be used for the selection of the cell e.g. by FACS enabling the isolation of a single cell clone.

In one embodiment the antibody is an antibody that specifically binds to an antigen. Thus, in one embodiment the antibody or the antibody encoding nucleic acid, respectively, is obtained from a B-cell that has been selected for that specifically binds to an antigen.

In one embodiment the antibody is a bivalent monospecific antibody. In one embodiment the antibody specifically binds to an antigen.

In one embodiment the antibody is a bivalent bispecific antibody. In one embodiment the antibody specifically binds to two different antigens or to two epitopes on the same antigen.

In one embodiment the antibody is a tetravalent bispecific antibody. In one embodiment the antibody specifically binds to two different antigens or to two epitopes on the same antigen.

In one embodiment the expression vector is a lentiviral (expression) vector.

One aspect as reported herein is a lentiviral vector library comprising two or more lentiviral particles each comprising an expression vector as reported herein, wherein the antibodies encoded by each vector differ in at least one amino acid from each other.

In one embodiment the vector library comprises of from 1,000 to 1,000,000 different expression vectors.

In one embodiment the antibodies encoded by the vectors of the vector library differ in at least one amino acid residue in the variable domains of the antibody.

In one embodiment the antibodies encoded by the vectors of the vector library differ in at least one amino acid residue in one of the CDRs of the antibody. In one embodiment the CDR is the heavy chain CDR3.

One aspect as reported herein is a eukaryotic cell comprising a bicistronic expression cassette as reported herein. In one embodiment the bicistronic expression cassette has been transduced into the cell.

One aspect as reported herein is a eukaryotic cell library comprising two or more eukaryotic cells each comprising a bicistronic expression cassette or a vector as reported herein, wherein the antibodies expressed by each cell differ in at least one amino acid from each other.

In one embodiment the eukaryotic cell library comprises of from 1,000 to 1,000,000 different mammalian cells.

In one embodiment the antibodies expressed by the cells of the eukaryotic cell library differ in at least one amino acid residue in the variable domains of the antibody.

In one embodiment the antibodies encoded by the eukaryotic cells of the eukaryotic cell library differ in at least one amino acid residue in one of the CDRs of the antibody. In one embodiment the CDR is the heavy chain CDR3.

One aspect as reported herein is a eukaryotic cell library comprising the vector library as reported herein.

In one embodiment the eukaryotic cells of the eukaryotic cell library express and display a single antibody.

In one embodiment the eukaryotic cells of the eukaryotic cell library display a single antibody.

In one embodiment the eukaryotic cell library is a population of eukaryotic cells expressing a library of antibodies wherein the encoding nucleic acid is derived from B-cells of an immunized animal. In one embodiment the B-cells are pre-selected for their specificity towards the antigen of interest.

In one embodiment the eukaryotic cell library is a population of eukaryotic cells wherein each cell comprises a first expression cassette encoding a full length antibody that specifically binds to a first antigen and a second expression cassette encoding a full length antibody that specifically binds to a second antigen.

In one embodiment the eukaryotic cell library is a population of eukaryotic cells wherein each cell comprises a first expression cassette encoding a first full length antibody light chain and a first full length antibody heavy chain binding to a first antigen and a second expression cassette encoding a second full length antibody light chain and a second full length antibody heavy chain that specifically binds to a second antigen.

In one embodiment the eukaryotic cell library is a population of eukaryotic cells wherein each cell comprises an expression cassette encoding a first full length antibody heavy chain that specifically binds to a first antigen and a second full length antibody heavy chain that specifically binds to a second antigen, wherein the eukaryotic cell expresses a common light chain.

In one embodiment the first full length antibody heavy chain comprises a hole mutation and the second antibody heavy chain comprises a knob mutation.

In one embodiment the first full length antibody light chain comprises as constant domain a CH1 domain and the first full length antibody heavy chain comprises as first constant domain a CL domain, or the second full length antibody light chain comprises as constant domain a CH1 domain and the second full length antibody heavy chain comprises as first constant domain a CL domain.

In one embodiment the expression vector library is obtained by randomization of one or more amino acids residues in one or more CDRs of a parent expression vector.

In one embodiment the expression vector library is obtained by combination of two different half antibodies.

One aspect as reported herein is a method for the isolation or selection of an antibody which specifically binds to one or more, especially two, antigen(s) of interest.

It has been found that the screening method reported herein can be performed in a “one antibody per cell” format, which is advantageous because it allows the screen to be completed in one single round of selection.

Herein is reported a method of generating, selecting, and/or isolating a cell expressing an antibody, which specifically binds to an antigen.

In one embodiment the antibody is a monoclonal full length antibody. In one embodiment the antibody is a bispecific monoclonal full length antibody.

The methods as reported herein allow cloning the variable regions of the antibody or the entire antibody from the selected cell.

One aspect as reported herein is a method to recombinantly produce antibodies selected with the method as reported herein.

In one embodiment the full length antibody comprises a constant region of human origin, especially of human IgG1, IgG2, or IgG4 class.

The methods as reported herein allow to recombinantly produce antibodies with desired specificity in a fully species specific form, especially as fully human antibodies.

One aspect as reported herein is a method of selecting a cell expressing an antibody which specifically binds to an antigen of interest comprising the steps of
(a) optionally selecting from a population of B-cells a sub-population of B-cells or a single B-cell or a clonal population of B-cells secreting an antibody that specifically binds one or more antigens,
(b) generating a lentiviral expression library, wherein each member of the lentiviral expression library encodes a variant of the antibody or the antibodies that specifically binds one or more antigens, by
   (i) generating a multitude of DNA molecules, wherein the generating comprises the step of amplifying a pool of DNA molecules from the sub-population of B-cells or the step of generating a library of DNA molecules from the DNA encoding a single antibody that specifically binds to one or two antigens of interest by randomization of the encoding nucleic acid sequence, and
   (ii) cloning the multitude of DNA molecules into a lentiviral expression vector comprising an EV71-IRES linked bicistronic expression cassette for the expression of a full length antibody light chain and a full length antibody heavy chain in a soluble as well as membrane bound form;
(c) transducing a population of eukaryotic cells with a population of lentiviral virus particles each comprising a member of the lentiviral expression library;
(d) displaying antibodies encoded by the lentiviral expression library on the surface of the eukaryotic mammalian cells; and
(e) isolating from the population of eukaryotic cells a cell, wherein the cell is selected for the capability of the antibody displayed on its surface to specifically bind the antigen or antigens of interest or a fragment or antigenic determinant thereof.

One aspect as reported herein is a method of selecting a cell expressing a bispecific antibody (which specifically binds to two antigens of interest) comprising the steps of
(a) generating a lentiviral expression library, wherein each member of the lentiviral expression library encodes a variant of the bispecific antibody, by
   (i) generating a multitude of DNA molecules from the DNA encoding a single bispecific antibody by randomization of the encoding nucleic acid sequence, and
   (ii) cloning the multitude of DNA molecules into a lentiviral expression vector comprising an EV71-IRES linked bicistronic expression cassette for the expression of a full length bispecific antibody as membrane bound form;
(b) transducing a population of eukaryotic cells with a population of lentiviral virus particles each comprising a member of the lentiviral expression library;
(c) displaying antibodies encoded by the lentiviral expression library on the surface of the eukaryotic mammalian cells; and
(d) isolating from the population of eukaryotic cells a cell, wherein the cell is selected for the capability of the antibody displayed on its surface to specifically bind the antigens of interest or a fragment or antigenic determinant thereof.

The use of a lentiviral expression library in combination with a lentiviral expression vector comprising an EV71-IRES linked bicistronic expression cassette for the expression of a full length antibody light chain and a full length antibody heavy chain in a soluble as well as membrane bound form allows for a high screening efficiency.

In one embodiment the method comprises generating a multitude of DNA molecules encoding antibodies, the generating a multitude of DNA molecules comprising the steps of:
(1) amplifying from a sub-population of B-cells a first pool of DNA molecules encoding heavy chain variable regions (HCVRs); and
(2) amplifying from the sub-population of B-cells a second pool of DNA molecules encoding light chain variable regions (LCVRs);
(3) cloning a combination of the multitude of DNA molecule encoding LCVRs and of the multitude of DNA molecules encoding HCVRs into a lentiviral expression vector comprising an EV71-IRES linked bicistronic expression cassette for the expression of a full length antibody light chain and a full length antibody heavy chain simultaneously in a soluble as well as membrane bound form.

In one embodiment the method comprises generating a multitude of DNA molecules encoding antibodies that specifically bind to one or two antigens of interest, the generating a multitude of DNA molecules comprising the steps of:
(1) amplifying from a single B-cell or a clonal population of B-cells a DNA molecule encoding the HCVR and a DNA molecule encoding the LCVR, and
(2) randomization the DNA molecule encoding the HCVR and/or the DNA molecule encoding the LCVR by randomizing at least one codon and thereby generating a multitude of DNA molecules encoding the HCVR and a multitude of DNA molecules encoding the LCVR;
(3) cloning a combination of the randomized multitude of DNA molecule encoding LCVRs and of the multitude of DNA molecules encoding HCVRs into a lentiviral expression vector comprising an EV71-IRES linked bicistronic expression cassette for the expression of a full length antibody light chain and a full length antibody heavy chain simultaneously in a soluble as well as membrane bound form.

In one embodiment the method comprises the generating of a lentiviral expression library, the generating comprising the steps of:
(i) generating a multitude of DNA molecules encoding antibodies, the generating comprising the steps of:
   (1) isolating mRNA from the sub-population of B-cells;
   (2) transcribing the mRNA to cDNA;
   (3) amplifying from the cDNA a first pool of DNA molecules using a first mixture of oligonucleotides comprising at least two oligonucleotides capable of amplifying HCVR coding regions; and
   (4) amplifying from the cDNA a second pool of DNA molecules using a second mixture of oligonucleotides comprising at least two oligonucleotides capable of amplifying LCVR coding regions;
(ii) cloning a pair of DNA molecules of the first and the second pool of DNA molecules into a lentiviral expression vector comprising an EV71-IRES linked bicistronic expression cassette for the expression of a full length antibody light chain and a full length antibody heavy chain simultaneously in a soluble as well as membrane bound form.

In one embodiment the method comprises the generating of a lentiviral expression library, the generating comprising the steps of:
(i) generating a multitude of DNA molecules encoding antibodies that specifically binds to one or two antigens, the generating comprising the steps of:
   (1) isolating mRNA from a single B-cell or a clonal population of B-cells;
   (2) transcribing the mRNA to cDNA;
   (3) amplifying from the cDNA a first DNA molecule using a first mixture of oligonucleotides comprising at least two oligonucleotides capable of amplifying HCVR coding region; and
   (4) amplifying from the cDNA a second DNA molecule using a second mixture of oligonucleotides comprising at least two oligonucleotides capable of amplifying LCVR coding region;
   (5) randomizing the first and/or the second DNA molecule and thereby generating a first pool of DNA molecules and a second pool of DNA molecules,
(ii) cloning a pair of DNA molecules of the first and the second pool of DNA molecules into a lentiviral expression vector comprising an EV71-IRES linked bicistronic expression cassette for the expression of a full length antibody light chain and a full length antibody heavy chain simultaneously in a soluble as well as membrane bound form.

One aspect as reported herein is a method of selecting a cell expressing a bispecific antibody (which specifically binds to two antigens of interest) comprising the steps of
(a) generating a lentiviral expression library, wherein each member of the lentiviral expression library encodes variants of the heavy chains of a bispecific antibody, by
   (i) generating a multitude of DNA molecules from the DNA encoding the heavy chains of a single bispecific antibody by randomization of the encoding nucleic acid sequence, and
   (ii) cloning the multitude of DNA molecules into a lentiviral expression vector comprising an EV71-IRES linked bicistronic expression cassette for the expression of the two heavy chains of the bispecific antibody, wherein the nucleic acid downstream of the EV71-IRES encodes an antibody heavy chain with a C-terminal transmembrane domain;
(b) transducing a population of eukaryotic cells expressing an antibody light chain that can form an antigen binding site with any of the antibody heavy chains with a population of lentiviral virus particles each comprising a member of the lentiviral expression library;
(c) displaying antibodies encoded by the lentiviral expression library on the surface of the eukaryotic mammalian cells; and
(d) isolating from the population of eukaryotic cells a cell, wherein the cell is selected for the capability of the antibody displayed on its surface to specifically bind the antigens of interest or a fragment or antigenic determinant thereof.

One aspect as reported herein is a lentiviral expression vector for displaying a full length antibody on the surface of a eukaryotic cell.

In one embodiment the expression vector comprises DNA elements encoding a signal peptide, an EV71-IRES, a transmembrane region and, optionally, a detection tag.

In one embodiment the expression vector comprises a restriction site allowing the cloning, especially the orientation specific cloning, of DNA molecules encoding a full length antibody heavy chain and a full length antibody light chain into the expression vector.

One aspect as reported herein is an expression library comprising the expression vector as reported herein.

One aspect as reported herein is a eukaryotic cell comprising the expression vector as reported herein or comprising at least one member of the expression library as reported herein.

Monoclonal antibodies produced by the method as reported herein can be used for research purposes, diagnostic purposes or the treatment of diseases.

In one embodiment the eukaryotic cell is a mammalian cell or a yeast cell. In one embodiment the mammalian cell is a CHO cell or a HEK cell.

One aspect as reported herein is a method of selecting a cell expressing an antibody comprising the steps of
(a) generating a population of eukaryotic cells by transduction with a population of lentiviral virus particles, whereby each cell of the population of cells displays a membrane-bound full length antibody which is encoded by the lentiviral nucleic acid, and which specifically binds to one or more antigens or one or more epitopes on the same antigen, and
(b) selecting from the population of eukaryotic cells a cell depending on the properties of the displayed membrane-bound full length antibody,
whereby each lentiviral virus particle of the population of lentiviral virus particles comprises a bicistronic expression cassette comprising the EV71-IRES for the expression of the membrane-bound antibody.

In one embodiment each bicistronic expression cassette of the lentiviral virus particle of the population of lentiviral virus particles encodes a different variant of a parent antibody, which specifically binds to one or more antigens or one or more epitopes on the same antigen.

In one embodiment the bicistronic expression cassettes comprises in 5′- to 3′-direction
a promoter,
a first nucleic acid encoding a full length antibody light chain,
the EV71-IRES,
a second nucleic acid encoding a full length antibody heavy chain,
a spliceable intron, and
a transmembrane domain or a GPI-anchor.

In one embodiment each cell of the population of eukaryotic cells displays a membrane-bound full length antibody and secretes a full length antibody.

In one embodiment each cell of the population of eukaryotic cells displays and secretes a single full length antibody.

In one embodiment the antibody is a bispecific antibody.

One aspect as reported herein is a lentiviral vector comprising a bicistronic expression cassette, which comprises in 5′- to 3′-direction
a promoter,
a first nucleic acid encoding a full length antibody light chain,
the EV71-IRES,
a second nucleic acid encoding a full length antibody heavy chain,
an alternatively spliceable intron for the simultaneous production of membrane-bound antibody and a secreted antibody, and
a transmembrane domain or a GPI-anchor.

One aspect as reported herein is a lentiviral vector comprising a bicistronic expression cassette, which comprises in 5′- to 3′-direction
a promoter,
a first nucleic acid encoding a first full length antibody heavy chain,
the EV71-IRES,
a second nucleic acid encoding in 5′- to 3′-direction a second full length antibody heavy chain and a transmembrane domain or a GPI-anchor.

One aspect as reported herein is the use of a lentiviral vector according to previous aspects for the generation of a population of eukaryotic cells displaying and secreting or displaying a full length antibody.

One aspect as reported herein is a method of selecting a cell expressing a bispecific antibody comprising the steps:
(a) generating a population of eukaryotic cells by transduction with a population of lentiviral virus particles, whereby each of the lentiviral virus particles comprises a bicistronic expression cassette, which comprises a first heavy chain variable domain encoding nucleic acid in a hole- or knob-locus, which is upstream of the EV71-IRES, and a second heavy chain variable domain encoding nucleic acid in the respective other locus, which is downstream of the EV71-IRES, whereby the first heavy chain variable domain binds to a first antigen and the second variable domain binds to a second antigen, whereby the first antigen and the second antigen can be the same or different, whereby the eukaryotic cell expresses a common light chain, whereby one or both of the heavy chains further comprise a transmembrane domain at their C-terminus, and
(b) selecting from the population of eukaryotic cells a cell depending on the properties of the displayed membrane-bound full length bispecific antibody.

In one embodiment only the heavy chain downstream of the EV71-IRES comprises a transmembrane domain at its C-terminus.

One aspect as reported herein is a method of selecting a cell secreting a bispecific antibody comprising the steps:
(a) generating a population of eukaryotic cells by transduction with a population of lentiviral virus particles, whereby each of the lentiviral virus particles comprises a bicistronic expression cassette encoding a secreted bispecific antibody, which comprises a first heavy chain variable domain encoding nucleic acid in a hole- or knob-locus upstream of the EV71-IRES and a second heavy chain variable domain encoding nucleic acid in the respective other locus downstream of the EV71-IRES, whereby the first heavy chain variable domain binds to a first antigen and the second variable domain binds to a second antigen, whereby the first antigen and the second antigen can be the same or different, whereby the eukaryotic cell expresses a common light chain,
(b) selecting from the population of eukaryotic cells a cell depending on the properties of the secreted full length bispecific antibody.

In one embodiment the method comprises as first step:
immunizing a transgenic animal with an antigen of interest, wherein the B-cells of the experimental animal express the same light chain.

In one embodiment the method comprises the step:
selecting the B-cells of the immunized experimental animal by bulk sorting by FACS.

In one embodiment the method comprises the step:
obtaining the heavy chain encoding nucleic acid of each B-cell by individual PCR amplification by two separate/sequential polymerase chain reactions introducing unique restriction sites to enable directed cloning into a shuttle vector/lentiviral expression vector.

In one embodiment the method comprises the step:
performing a PCR of the complete first heavy chain encoding nucleic acid and the variable domain of the second heavy chain encoding nucleic acid (2.2 kbp) including the EV71-IRES and cloning into second shuttle vector without transmembrane-domain with the removal of the transmembrane-domain of the first heavy chain by restriction cutting and religation of the vector.

All embodiments as reported herein shall refer to all aspects of the invention and may be combined in any possible combination.

### DETAILED DESCRIPTION OF THE INVENTION

Herein is reported a selection method using the expression of full length antibodies in their natural environment, i.e. the secretory pathway of mammalian cells, that ensures that all the cellular components normally involved in antibody synthesis and processing (folding, disulfide bond formation, glycosylation etc.) are available in a physiological form and concentration.

### General Aspects

As known to a person skilled in the art enables the use of recombinant DNA technology the production of numerous derivatives of a nucleic acid and/or polypeptide. Such derivatives can, for example, be modified in one individual or several positions by substitution, alteration, exchange, deletion, or insertion. The modification or derivatization can, for example, be carried out by means of site directed mutagenesis. Such modifications can easily be carried out by a person skilled in the art (see e.g. Sambrook, J., et al., Molecular Cloning: A Laboratory Manual, Cold Spring Harbor Laboratory Press, New York, USA (1999)). The use of recombinant technology enables a person skilled in the art to transform various host cells with heterologous nucleic acid(s). Although the transcription and translation, i.e. expression, machinery of different cells use the same elements, cells belonging to different species may have among other things a different so-called codon usage. Thereby identical polypeptides (with respect to amino acid sequence) may be encoded by different nucleic acid(s). Also, due to the degeneracy of the genetic code, different nucleic acids may encode the same polypeptide.

The use of recombinant DNA technology enables the production of numerous derivatives of a nucleic acid and/or polypeptide. Such derivatives can, for example, be modified in one individual or several positions by substitution, alteration, exchange, deletion, or insertion. The modification or derivatization can, for example, be carried out by means of site directed mutagenesis. Such modifications can easily be carried out by a person skilled in the art (see e.g. Sambrook, J., et al., Molecular Cloning: A Laboratory Manual, Cold Spring Harbor Laboratory Press, New York, USA (1999); Hames, B. D., and Higgins, S. J., Nucleic acid hybridization—a practical approach, IRL Press, Oxford, England (1985)).

The use of recombinant technology enables the transformation of various host cells with heterologous nucleic acid(s). Although the transcription and translation, i.e. expression, machinery of different cells use the same elements, cells belonging to different species may have among other things a different so-called codon usage. Thereby identical polypeptides (with respect to amino acid sequence) may be encoded by different nucleic acid(s). Also, due to the degeneracy of the genetic code, different nucleic acids may encode the same polypeptide.

### DEFINITIONS

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, and multispecific antibodies (e.g., bispecific antibodies).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “expression” as used herein refers to transcription and/or translation processes occurring within a cell. The level of transcription of a nucleic acid sequence of interest in a cell can be determined on the basis of the amount of corresponding mRNA that is present in the cell. For example, mRNA transcribed from a sequence of interest can be quantitated by RT-PCR or by Northern hybridization (see Sambrook et al., 1999, supra). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook et al., 1999, supra).

An “expression cassette” refers to a construct that contains the necessary regulatory elements, such as promoter and polyadenylation site, for expression of at least the contained nucleic acid in a cell.

An “expression vector” is a nucleic acid providing all required elements for the expression of the comprised structural gene(s) in a host cell. Typically, an expression plasmid comprises a prokaryotic plasmid propagation unit, e.g. for E. coli, comprising an origin of replication, and a selectable marker, an eukaryotic selection marker, and one or more expression cassettes for the expression of the structural gene(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A., et al., Sequences of Proteins of Immunological Interest, 5th ed., Public Health Service, National Institutes of Health, Bethesda, Md. (1991), NIH Publication 91-3242.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc-region as defined herein.

A “gene” denotes a nucleic acid which is a segment e.g. on a chromosome or on a plasmid which can affect the expression of a peptide, polypeptide, or protein. Beside the coding region, i.e. the structural gene, a gene comprises other functional elements e.g. a signal sequence, promoter(s), introns, and/or terminators.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat, E. A., et al., Sequences of Proteins of Immunological Interest, 5th ed. Public Health Service, National Institutes of Health, Bethesda, Md. (1991), NIH Publication 91-3242). With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3 (Almagro, J. C. and Fransson, J., Front. Biosci. 13 (2008) 1619-1633). Unless otherwise indicated, HVR residues and other residues in the variable domain (e.g., FR residues) are numbered herein according to Kabat et al., supra.

An “internal ribosome entry site” or “IRES” describes a sequence which functionally promotes translation initiation independent from the gene 5′ of the-IRES and allows two cistrons (open reading frames) to be translated from a single transcript in an animal cell. The IRES provides an independent ribosome entry site for translation of the open reading frame immediately downstream (downstream is used interchangeably herein with 3′) of it. Unlike bacterial mRNA which can be polycistronic, i.e. encode several different polypeptides that are translated sequentially from the mRNAs, most mRNAs of animal cells are monocistronic and code for the synthesis of only one protein. With a polycistronic transcript in a eukaryotic cell, translation would initiate from the 5′ most translation initiation site, terminate at the first stop codon, and the transcript would be released from the ribosome, resulting in the translation of only the first encoded polypeptide in the mRNA. In a eukaryotic cell, a polycistronic transcript having an IRES operably linked to the second or subsequent open reading frame in the transcript allows the sequential translation of that downstream open reading frame to produce the two or more polypeptides encoded by the same transcript. The use of IRES elements in vector construction has been previously described, see, e.g., Pelletier, J., et al., Nature 334 (1988) 320-325; Jang, S. K., et al., J. Virol. 63 (1989) 1651-1660; Davies, M. V., et al., J. Virol. 66 (1992) 1924-1932; Adam, M. A., et al., J. Virol. 65 (1991) 4985-4990; Morgan, R. A., et al. Nucl. Acids Res. 20 (1992) 1293-1299; Sugimoto, Y., et al., Biotechnology 12 (1994) 694-698; Ramesh, N., et al., Nucl. Acids Res. 24 (1996) 2697-2700; and Mosser, D. D., et al., BioTechniques 22 (1997) 150-152).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “nucleic acid” as used herein, refers to a polymeric molecule consisting of individual nucleotides (also called bases) a, c, g, and t (or u in RNA), for example to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed (e.g. by mutagenesis), deleted, or added. A nucleic acid can either be isolated, or integrated in another nucleic acid, e.g. in an expression cassette, a plasmid, or the chromosome of a host cell. A nucleic acid is likewise characterized by its nucleic acid sequence consisting of individual nucleotides.

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

A “nucleic acid” as used herein, also refers to a naturally occurring or partially or fully non-naturally occurring nucleic acid encoding a polypeptide which can be produced recombinantly. The nucleic acid can be build up of DNA-fragments which are either isolated or synthesized by chemical means. The nucleic acid can be integrated into another nucleic acid, e.g. in an expression plasmid or the genome/chromosome of a eukaryotic host cell. Plasmid includes shuttle and expression plasmids. Typically, the plasmid will also comprise a prokaryotic propagation unit comprising an origin of replication (e.g. the ColE1 origin of replication) and a selectable marker (e.g. ampicillin or tetracycline resistance gene), for replication and selection, respectively, of the plasmid in prokaryotes.

“Operably linked” refers to a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader and a polypeptide, contiguous and in (reading) frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within or downstream of coding sequences and at considerable distance from the promoter. A polyadenylation site is operably linked to a coding sequence if it is located at the downstream end of the coding sequence such that transcription proceeds through the coding sequence into the polyadenylation sequence. A translation stop codon is operably linked to an exonic nucleic acid sequence if it is located at the downstream end (3′ end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

A “polycistronic transcription unit” is a transcription unit in which more than one structural gene is under the control of the same promoter.

The term “polyadenylation signal” (polyA signal) as used within this application denotes a nucleic acid sequence used to induce cleavage and polyadenylation of primary transcripts of a specific nucleic acid sequence segment. The 3′ untranslated region comprising a polyadenylation signal can be selected from the group consisting of the 3′ untranslated region comprising a polyadenylation signals derived from SV40, the gene for bovine growth hormone (bGH), immunoglobulin genes, and the thymidine kinase gene (tk, e.g. Herpes Simplex thymidine kinase polyadenylation signal).

A “promoter” refers to a polynucleotide sequence that controls transcription of a gene/structural gene or nucleic acid sequence to which it is operably linked. A promoter includes signals for RNA polymerase binding and transcription initiation.

The promoters used will be functional in the cell type of the host cell in which expression of the selected sequence is contemplated. A large number of promoters including constitutive, inducible and repressible promoters from a variety of different sources, are well known in the art (and identified in databases such as GenBank) and are available as or within cloned polynucleotides (from, e.g., depositories such as ATCC as well as other commercial or individual sources).

A “promoter” comprises a nucleotide sequence that directs the transcription of a structural gene. Typically, a promoter is located in the 5′ non-coding or untranslated region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee, R. E., et al., Mol. Endocrinol. 7 (1993) 551), cyclic AMP response elements (CREs), serum response elements (SREs; Treisman, R., Seminars in Cancer Biol. 1 (1990) 47), glucocorticoid response elements (GREs), and binding sites for other transcription factors, such as CRE/ATF (O'Reilly, M. A., et al., J. Biol. Chem. 267 (1992) 19938), AP2 (Ye, J., et al., J. Biol. Chem. 269 (1994) 25728), SP1, cAMP response element binding protein (CREB; Loeken, M. R., Gene Expr. 3 (1993) 253) and octamer factors (see, in general, Watson et al., (eds.), Molecular Biology of the Gene, 4th ed. (The Benjamin/Cummings Publishing Company, Inc. (1987)), and Lemaigre, F. P. and Rousseau, G. G., Biochem. J. 303 (1994) 1-14). If a promoter is an inducible promoter, then the rate of transcription increases in response to an inducing agent. In contrast, the rate of transcription is not regulated by an inducing agent if the promoter is a constitutive promoter. Repressible promoters are also known. For example, the c-fos promoter is specifically activated upon binding of growth hormone to its receptor on the cell surface. Tetracycline (tet) regulated expression can be achieved by artificial hybrid promoters that consist e.g. of a CMV promoter followed by two Tet-operator sites. The Tet-repressor binds to the two Tet-operator sites and blocks transcription. Upon addition of the inducer tetracycline, Tet-repressor is released from the Tet-operator sites and transcription proceeds (Gossen, M. and Bujard, H., PNAS 89 (1992) 5547-5551). For other inducible promoters including metallothionein and heat shock promoters, see, e.g., Sambrook et al. (supra) and Gossen et al., Curr. Opin. Biotech. 5 (1994) 516-520. Among the eukaryotic promoters that have been identified as strong promoters for high-level expression are the SV40 early promoter, adenovirus major late promoter, mouse metallothionein-I promoter, Rous sarcoma virus long terminal repeat, Chinese hamster elongation factor 1 alpha (CHEF-1, see e.g. U.S. Pat. No. 5,888,809), human EF-1 alpha, ubiquitin, and human cytomegalovirus immediate early promoter (CMV IE).

The “promoter” can be constitutive or inducible. An enhancer (i.e., a cis-acting DNA element that acts on a promoter to increase transcription) may be necessary to function in conjunction with the promoter to increase the level of expression obtained with a promoter alone, and may be included as a transcriptional regulatory element. Often, the polynucleotide segment containing the promoter will include enhancer sequences as well (e.g., CMV or SV40).

The term “transcription terminator” denotes a DNA sequence of 50-750 base pairs in length which gives the RNA polymerase the signal for termination of the mRNA synthesis. Very efficient (strong) terminators at the 3′ end of an expression cassette are advisable to prevent the RNA polymerase from reading through particularly when using strong promoters. Inefficient transcription terminators can lead to the formation of an operon-like mRNA which can be the reason for an undesired, e.g. plasmid-coded, gene expression.

Within the scope of the present invention, transfected cells may be obtained with substantially any kind of transfection method known in the art. For example, the nucleic acid may be introduced into the cells by means of electroporation or microinjection. Alternatively, lipofection reagents such as FuGENE 6 (Roche Diagnostics GmbH, Germany), X-tremeGENE (Roche Diagnostics GmbH, Germany), and LipofectAmine (Invitrogen Corp., USA) may be used. Still alternatively, the nucleic acid may be introduced into the cell by appropriate viral vector systems based on retroviruses, lentiviruses, adenoviruses, or adeno-associated viruses (Singer, 0., Proc. Natl. Acad. Sci. USA 101 (2004) 5313-5314).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt, T. J., et al., Kuby Immunology, 6th ed., W.H. Freeman and Co., N.Y. (2007), page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano, S., et al., J. Immunol. 150 (1993) 880-887; Clackson, T., et al., Nature 352 (1991) 624-628).

The term “vector” denotes a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “animal” denotes an organism comprising an immune system capable of producing antibodies. In one embodiment the animal is selected from fish, amphibians, birds, reptiles, and mammals, especially artiodactyls, rodents and primates. In one embodiment the animal is selected from the group consisting of sheep, elk, deer, donkey, mule deer, mink, horse, cattle, pig, goat, dog, cat, rat, hamster, guinea pig, and mouse. In one embodiment the animal is a mouse, a rat or, a primate. In one embodiment the animal is a non-human primate or a human. In one embodiment the animal is a transgenic animal with a human immunoglobulin locus.

A light chain variable region (LCVR) is encoded by a rearranged nucleic acid molecules derived from the germline genes of the respective animal. A light chain variable region is either a kappa LCVR or a lambda LCVR.

In one embodiment the light chain variable region is a human kappa LCVRs. In one embodiment the light chain variable region is a light chain variable region which is encoded by a nucleic acid (DNA) which can be amplified from human B-cells or the B-cells of a transgenic animal with a human immunoglobulin locus using a primer combination of one or more of SEQ ID NO: 12 to 18 with SEQ ID NO: 19, and PCR conditions as described in Example 11.

In one embodiment the light chain variable region is a human lambda LCVR. In one embodiment the light chain variable region is a variable region which is encoded by a nucleic acid (DNA) which can be amplified from human B-cells or the B-cells of a transgenic animal comprising a human immunoglobulin locus using a primer combination of one or more of SEQ ID NO: 20 to 27 with SEQ ID NO: 28, and PCR conditions as described in Example 11.

A heavy chain variable region (HCVR) is encoded by a rearranged nucleic acid molecules derived from the germline genes of the respective animal. In one embodiment the heavy chain variable region is a human heavy chain variable region. In one embodiment the heavy chain variable region is a heavy chain variable region which is encoded by a nucleic acid (DNA) which can be amplified from human B-cells or the B-cells of a transgenic animal comprising a human immunoglobulin locus using a primer combination of one or more of SEQ ID NO: 1 to 4 with SEQ ID NO: 5 and PCR conditions described in Example 11.

A heavy chain variable region (HCVR) is encoded by a rearranged nucleic acid molecules derived from the germline genes of the respective animal. In one embodiment the heavy chain variable region is a human heavy chain variable region. In one embodiment the heavy chain variable region is a heavy chain variable region which is encoded by a nucleic acid (DNA) which can be amplified from human B-cells or the B-cells of a transgenic animal comprising a human immunoglobulin locus using a primer combination of one or more of SEQ ID NO: 6 to 10 with SEQ ID NO: 11 and PCR conditions described in Example 11.

### Antibody

The methods provided herein are for the production of recombinant monoclonal antibodies. An antibody can be of various structures, such as but not limited to monospecific antibodies, multispecific antibodies (e.g., bispecific antibodies), monovalent antibodies, and multivalent antibodies (e.g. bivalent antibodies).

In certain embodiments, the antibody is a chimeric antibody. Certain chimeric antibodies are described, e.g., in U.S. Pat. No. 4,816,567; and Morrison, S. L., et al., Proc. Natl. Acad. Sci. USA 81 (1984) 6851-6855. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro, J. C. and Fransson, J., Front. Biosci. 13 (2008) 1619-1633, and are further described, e.g., in Riechmann, I., et al., Nature 332 (1988) 323-329; Queen, C., et al., Proc. Natl. Acad. Sci. USA 86 (1989) 10029-10033; U.S. Pat. No. 5,821,337, U.S. Pat. No. 7,527,791, U.S. Pat. No. 6,982,321, and U.S. Pat. No. 7,087,409; Kashmiri, S. V., et al., Methods 36 (2005) 25-34 (describing SDR (a-CDR) grafting); Padlan, E. A., Mol. Immunol. 28 (1991) 489-498 (describing “resurfacing”); Dall′Acqua, W. F., et al., Methods 36 (2005) 43-60 (describing “FR shuffling”); and Osbourn, J., et al., Methods 36 (2005) 61-68 and Klimka, A., et al., Br. J. Cancer 83 (2000) 252-260 (describing the “guided selection” approach to FR shuffling).

Human framework regions that may be used for humanization include but are not limited to: framework regions selected using the “best-fit” method (see, e.g., Sims, M. J., et al., J. Immunol. 151 (1993) 2296-2308); framework regions derived from the consensus sequence of human antibodies of a particular subgroup of light or heavy chain variable regions (see, e.g., Carter, P., et al., Proc. Natl. Acad. Sci. USA 89 (1992) 4285-4289; and Presta, L. G., et al., J. Immunol. 151 (1993) 2623-2632); human mature (somatically mutated) framework regions or human germline framework regions (see, e.g., Almagro, J. C. and Fransson, J., Front. Biosci. 13 (2008) 1619-1633); and framework regions derived from screening FR libraries (see, e.g., Baca, M., et al., J. Biol. Chem. 272 (1997) 10678-10684 and Rosok, M. J., et al., J. Biol. Chem. 271 (1996) 22611-22618).

In certain embodiments, the antibody is a human antibody. Human antibodies can be produced using various techniques known in the art. Human antibodies are described generally in van Dijk, M. A. and van de Winkel, J. G., Curr. Opin. Pharmacol. 5 (2001) 368-374 and Lonberg, N., Curr. Opin. Immunol. 20 (2008) 450-459.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, N., Nat. Biotech. 23 (2005) 1117-1125 and also, e.g., U.S. Pat. No. 6,075,181 and U.S. Pat. No. 6,150,584 describing XENOMOUSE™ technology; U.S. Pat. No. 5,770,429 describing HUMAB® technology; U.S. Pat. No. 7,041,870 describing K-M MOUSE® technology, and US 2007/0061900, describing VELoCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described (see, e.g., Kozbor, D., J. Immunol. 133 (1984) 3001-3005; Brodeur, B. R., et al., Monoclonal Antibody Production Techniques and Applications, Marcel Dekker, Inc., New York (1987), pp. 51-63; and Boerner, P., et al., J. Immunol. 147 (1991) 86-95). Human antibodies generated via human B-cell hybridoma technology are also described in Li, J., et al., Proc. Natl. Acad. Sci. USA 103 (2006) 3557-3562. Additional methods include those described, for example, in U.S. Pat. No. 7,189,826 (describing production of monoclonal human IgM antibodies from hybridoma cell lines) and Ni, J., Xiandai Mianyixue 26 (2006) 265-268 (describing human-human hybridomas). Human hybridoma technology (Trioma technology) is also described in Vollmers, H. P. and Brandlein, S., Histology and Histopathology 20 (2005) 927-937 and Vollmers, H. P. and Brandlein, S., Methods and Findings in Experimental and Clinical Pharmacology 27 (2005) 185-191.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom, H. R., et al., Methods Mol. Biol. 178 (2002) 1-37 and further described, e.g., in the McCafferty, J., et al., Nature 348 (1990) 552-554; Clackson, T., et al., Nature 352 (1991) 624-628; Marks, J. D., et al., J. Mol. Biol. 222 (1992) 581-597; Marks, J. D. and Bradbury, A., Methods in Molecular Biology 248 (2003) 161-175; Sidhu, S. S., et al., J. Mol. Biol. 338 (2004) 299-310; Lee, C. V., et al., J. Mol. Biol. 340 (2004) 1073-1093; Fellouse, F. A., Proc. Natl. Acad. Sci. USA 101 (2004) 12467-12472; and Lee, C. V., et al., J. Immunol. Methods 284 (2004) 119-132.

In certain phage display methods, repertoires of VH and VL genes are separately cloned by polymerase chain reaction (PCR) and recombined randomly in phage libraries, which can then be screened for antigen-binding phage as described in Winter, G., et al., Ann. Rev. Immunol. 12 (1994) 433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths, A. D., et al., EMBO J. 12 (1993) 725-734. Finally, naive libraries can also be made synthetically by cloning non-rearranged V-gene segments from stem cells, and using PCR primers containing random sequence to encode the highly variable CDR3 regions and to accomplish rearrangement in vitro, as described by Hoogenboom, H. R. and Winter, G., J. Mol. Biol. 227 (1992) 381-388. Patent publications describing human antibody phage libraries include, for example, U.S. Pat. No. 5,750,373, and US 2005/0079574, US 2005/0119455, US 2005/0266000, US 2007/0117126, US 2007/0160598, US 2007/0237764, US 2007/0292936, and US 2009/0002360.

Antibodies or antibody fragments isolated from human antibody libraries are considered human antibodies or human antibody fragments herein.

In certain embodiments, the antibody is a multispecific antibody, e.g. a bispecific antibody. Multispecific antibodies are monoclonal antibodies that have binding specificities for at least two different sites. In certain embodiments, one of the binding specificities is for a first antigen and the other is for a different second antigen. In certain embodiments, bispecific antibodies may bind to two different epitopes of the same antigen. Bispecific antibodies may also be used to localize cytotoxic agents to cells which express the antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553; using “diabody” technology for making bispecific antibody fragments (see, e.g., Holliger, P., et al., Proc. Natl. Acad. Sci. USA 90 (1993) 6444-6448); and using single-chain Fv (sFv) dimers (see, e.g., Gruber, M., et al., J. Immunol. 152 (1994) 5368-5374); and preparing trispecific antibodies as described, e.g., in Tutt, A., et al., J. Immunol. 147 (1991) 60-69).

Engineered antibodies with three or more functional antigen binding sites, including “Octopus antibodies”, are also included herein (see, e.g. US 2006/0025576).

The antibody or fragment can also be a multispecific antibody as described in WO 2009/080251, WO 2009/080252, WO 2009/080253, WO 2009/080254, WO 2010/112193, WO 2010/115589, WO 2010/136172, WO 2010/145792, or WO 2010/145793.

### Methods

In certain embodiments, the methods provided herein are used to alter, i.e. to increase or decrease, the extent to which the antibody is glycosylated.

Where the antibody comprises an Fc-region, the carbohydrate attached thereto may be altered. Native antibodies produced by mammalian cells typically comprise a branched, biantennary oligosaccharide that is generally attached by an N-linkage to Asn297 of the CH2 domain of the Fc-region (see, e.g., Wright, A. and Morrison, S. L., TIBTECH 15 (1997) 26-32). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, the method provided results in the production of antibodies having a carbohydrate structure that lacks fucose attached (directly or indirectly) to an Fc-region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc-region (EU numbering according to Kabat of Fc-region residues); however, Asn297 may also be located about ±3 amino acids upstream or downstream of position 297, i.e., between positions 294 and 300, due to minor sequence variations in antibodies. Such fucosylation variants may have improved ADCC function (see, e.g., US 2003/0157108; US 2004/0093621). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki, A., et al., J. Mol. Biol. 336 (2004) 1239-1249; Yamane-Ohnuki, N., et al., Biotech. Bioeng. 87 (2004) 614-622. Examples of cell lines capable of producing defucosylated antibodies include Lec13 CHO cells deficient in protein fucosylation (Ripka, J., et al., Arch. Biochem. Biophys. 249 (1986) 533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki, N., et al., Biotech. Bioeng. 87 (2004) 614-622; Kanda, Y., et al., Biotechnol. Bioeng. 94 (2006) 680-688; and WO 2003/085107).

In certain embodiments, the methods provided can be used to produce antibodies with bisected oligosaccharides, e.g., in which a biantennary oligosaccharide attached to the Fc-region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc-region can also be produced. Such antibody variants may have improved CDC function. Such antibody variants are described, e.g., in WO 1997/30087; WO 1998/58964; and WO 1999/22764.

Antibodies may be produced using recombinant methods and compositions, e.g., as described in U.S. Pat. No. 4,816,567. Nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0) or human embryonic kidney cells (HEK293). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc-region effector function are not needed. For expression of antibody fragments and polypeptides in bacteria, see, e.g., U.S. Pat. No. 5,648,237, U.S. Pat. No. 5,789,199, and U.S. Pat. No. 5,840,523; see also Charlton, K. A., In: Methods in Molecular Biology, Vol. 248, Lo, B. K. C. (ed.), Humana Press, Totowa, N.J. (2003), pp. 245-254, describing expression of antibody fragments in E. coli. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (see Gerngross, T. U., Nat. Biotech. 22 (2004) 1409-1414; and Li, H., et al., Nat. Biotech. 24 (2006) 210-215).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (see, e.g., U.S. Pat. No. 5,959,177, U.S. Pat. No. 6,040,498, U.S. Pat. No. 6,420,548, U.S. Pat. No. 7,125,978, and U.S. Pat. No. 6,417,429 (describing PLANTIBODIES™ technology for producing antibodies in transgenic plants)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR negative (DHFR(−)) CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

### SPECIFIC EMBODIMENTS OF THE INVENTION

Herein are reported methods for the selection of cells expressing antibodies with a desired specificity as well as methods for producing such antibodies.

Although it is possible to identify antibodies with various screening methods subsequent development at production scale can be hampered by constrains of protein expression, incorrect folding and/or incorrect posttranslational modifications as well as incorrect antibody assembly, such as homodimer formation.

In contrast to an antigen binding fragment has a full length antibody several additional features, such as prolonged serum half-live (weeks compared to hours or days), support of secondary immune functions, such as ADCC, CDC, FcRn binding

In one embodiment the antibody specifically binds to one antigen of interest.

In one embodiment the antibody specifically binds to two different antigens or to two different, non-overlapping epitopes on the same antigen.

Generally the antigen of interest is a protein antigen, a non-protein antigen or a hapten. The antigen of interest is in one embodiment selected from the group consisting of (a) antigen of a microorganism or of a pathogen, (b) tumor antigen, (c) self-antigen, and (d) allergen.

A tumor antigen is a compound, such as a peptide, associated with a tumor or cancer and which can be bound by an antibody. Tumor antigens can be prepared from cancer cells either by preparing crude extracts of cancer cells, for example, as described in Cohen, et al., Cancer Research, 54 (1994) 1055, by partially purifying the antigens, by recombinant technology or by de novo synthesis of known antigens. Tumor antigens include antigens that are antigenic portions of or are a whole tumor or cancer polypeptide. Such antigens can be isolated or prepared recombinantly or by any other means known in the art. Cancers or tumors include, but are not limited to, biliary tract cancer; brain cancer; breast cancer; cervical cancer; chorio carcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

The term “antigenic determinant” denotes the portion of an antigen that is specifically recognized by B-lymphocytes. B-lymphocytes respond to foreign antigenic determinants by antibody production.

With respect to an antibody displayed on a mammalian cell the specificity of the binding of an antigen is in one embodiment determined in a fluorescence assay essentially as set forth herein in Example 12, wherein the intensity of a fluorescence signal is correlated with the amount of antigen bound by a cell displaying the antibody. Antibodies displayed on mammalian cells are regarded as that specifically binds an antigen, when the intensity of the fluorescence signal is higher than the signal detected for control cells. In one embodiment the signal is at least two times higher than that of control cells.

The term “expression library” denotes a multitude of expression vectors of the same type, wherein individual expression vectors expresses a different antibody. In one embodiment the expression library is a viral expression library. In one embodiment the expression library is a lentiviral expression library.

The term “multiplicity of infection” (MOI) denotes the ratio between the number of infectious virus particles in a viral, especially a lentiviral, expression library and the number of cells exposed to the virus.

Herein is reported a method of generating, selecting, and/or isolating a cell expressing an antibody of desired specificity.

In more detail, the method comprises:

providing a nucleic acid encoding a full length antibody

In one embodiment the nucleic acid is obtained by selecting from a population of isolated B-cells a sub-population of B-cells by selecting B-cells for their capability of that specifically binds the antigen of interest.

In one embodiment the nucleic acid is obtained by selecting from a population of isolated B-cells a single B-cell by selecting a B-cell for its capability of that specifically binds the one or two antigens of interest.

In one embodiment the single B-cell is a clonal B-cell population.

In one embodiment the nucleic acid is obtained by amplifying the variable domain encoding nucleic acid from the isolated mRNA of a single B-cell or a clonal B-cell population and transcribing the amplified mRNA into cDNA.

### Generating a Lentiviral Expression Library

The lentiviral expression vector as reported herein and used in the methods as reported herein is a vector comprising a bicistronic expression cassette for the expression of a full length antibody in soluble and membrane-bound form. By providing a soluble and a membrane-bound form of the antibody at the same time the cell expressing the antibody can be selected based on the surface presented antibody and the antibody can be tested for e.g. binding specificity by using the secreted antibody.

It has been found that for expressing and displaying a full length antibody on a mammalian cell it is required to use a bicistronic expression construct that further comprises a spliceable nucleic acid in order to express a soluble and membrane-bound form from the same expression cassette.

Due to the fact that the size of the lentiviral expression vector is limited in order to be efficiently packaged in a viral particle and due to the fact that a full length antibody has to be expressed and presented the transmembrane encoding nucleic acid has also to be shortened and reduced in size.

The diversity of the lentiviral expression library can be generated by:
(i) In one embodiment the diversity of the lentiviral expression library is generated by using the HCVR and LCVR encoding nucleic acids obtained from a pool of B-cells producing an antibody that specifically binds to one antigen, or two different antigens, or that specifically binds to two different, non-overlapping epitopes of the same antigen.
(ii) In one embodiment the diversity of the lentiviral expression library is generated by using pairs of HCVR and LCVR encoding nucleic acids selected from pools of HCVR and LCVR encoding nucleic acids, which are obtained by randomizing at least one codon of the HCVR and the LCVR encoding nucleic acid obtained from a single B-cell producing an antibody that specifically binds to one antigen, or two different antigens, or that specifically binds to two different, non-overlapping epitopes of the same antigen.

In one embodiment the single B-cell is a clonal population of B-cells.

In one embodiment the at least one codon is in the CDR of the HCVR or the LCVR. In one embodiment the CDR is the CDR3. In one embodiment the CDR3 is the HCDR3.
(iii) In one embodiment the diversity of the lentiviral expression library is generated by using pairs of different HCVR encoding nucleic acids and a single LCVR encoding nucleic acid, whereby the different HCVR encoding nucleic acids are obtained by randomizing at least one codon of the HCVR encoding nucleic acid obtained from a single B-cell producing an antibody that specifically binds to one antigen, or two different antigens, or that specifically binds to two different, non-overlapping epitopes of the same antigen.

In one embodiment the single B-cell is a clonal population of B-cells.

In one embodiment the at least one codon is in the CDR of the HCVR. In one embodiment the CDR is the CDR3.
(iv) In one embodiment the diversity of the lentiviral expression library is generated by using pairs of different LCVR encoding nucleic acids and a single HCVR encoding nucleic acid, whereby the different LCVR encoding nucleic acids are obtained by randomizing at least one codon of the LCVR encoding nucleic acid obtained from a single B-cell producing an antibody that specifically binds to one antigen, or two different antigens, or that specifically binds to two different, non-overlapping epitopes of the same antigen.

In one embodiment the single B-cell is a clonal population of B-cells.

In one embodiment the at least one codon is in the CDR of the LCVR. In one embodiment the CDR is the CDR3.

In one embodiment the generating of the diversity of the lentiviral expression library comprises the steps of
(a):
   (i) isolating RNA from the sub-population of B-cells,
   (ii) transcribing the RNA to cDNA;
   (iii) amplifying from the cDNA a first pool of DNA molecules using a first mixture of oligonucleotides comprising at least two oligonucleotides capable of amplifying HCVR coding regions;
   (iv) amplifying from the cDNA a second pool of DNA molecules using a second mixture of oligonucleotides comprising at least two oligonucleotides capable of amplifying LCVR coding regions; and
   (v) providing pairs of one member of the first pool of DNA molecules and one member of the second pool of DNA molecules;
or (b):
   (i) isolating RNA from a single B-cell, or from a clonal population of B-cells,
   (ii) transcribing the RNA to cDNA;
   (iii) amplifying from the cDNA a first DNA molecule using a first mixture of oligonucleotides comprising at least two oligonucleotides capable of amplifying HCVR coding regions;
(iv) amplifying from the cDNA a second DNA molecule using a second mixture of oligonucleotides comprising at least two oligonucleotides capable of amplifying LCVR coding regions;
   (v) generating a first pool of DNA molecules by randomizing at least one codon of the first DNA molecule,
   (vi) generating a second pool of DNA molecules by randomizing at least one codon of the second DNA molecule, and
   (vii) providing pairs of one member of the first pool of DNA molecules and one member of the second pool of DNA molecules;
or (c):
   (i) isolating RNA from a single B-cell, or from a clonal population of B-cells,
   (ii) transcribing the RNA to cDNA;
   (iii) amplifying from the cDNA a first DNA molecule using a first mixture of oligonucleotides comprising at least two oligonucleotides capable of amplifying HCVR coding regions;
   (iv) amplifying from the cDNA a second DNA molecule using a second mixture of oligonucleotides comprising at least two oligonucleotides capable of amplifying LCVR coding regions;
   (v) generating a pool of DNA molecules by randomizing at least one codon of the first DNA molecule, and
   (vi) providing pairs of one member of the pool of DNA molecules and the second DNA molecule;
or (d):
   (i) isolating RNA from a single B-cell, or from a clonal population of B-cells,
   (ii) transcribing the RNA to cDNA;
   (iii) amplifying from the cDNA a first DNA molecule using a first mixture of oligonucleotides comprising at least two oligonucleotides capable of amplifying HCVR coding regions;
   (iv) amplifying from the cDNA a second DNA molecule using a second mixture of oligonucleotides comprising at least two oligonucleotides capable of amplifying LCVR coding regions;
   (v) generating a pool of DNA molecules by randomizing at least one codon of the second DNA molecule, and
   (vi) providing pairs of one member of the pool of DNA molecules and the first DNA molecule.

To produce a secreted polypeptide, the structural gene of interest includes a DNA segment that encodes a “signal sequence” or “leader peptide”. The signal sequence directs the newly synthesized polypeptide to and through the ER membrane where the polypeptide can be routed for secretion. The signal sequence is cleaved off by a signal peptidases during the protein crosses the ER membrane. As for the function of the signal sequence the recognition by the host cell's secretion machinery is essential. Therefore the used signal sequence has to be recognized by the host cell's proteins and enzymes of the secretion machinery.

The lentiviral expression vector used for the generation of the lentiviral expression library allows for the expression of a secreted and a membrane-bound form of the antibody. The membrane-bound form is expressed via the linking of the C-terminal constant domain of the antibody heavy chain to an alternatively spliceable nucleic acid (intron) and further an exon encoding a transmembrane or a signal peptide for a GPI-anchor.

The term “GPI-anchor” as used within this application denotes a posttranslational modification attached to a C-terminus of a polypeptide or protein. A “GPI-anchor” has a core structure comprising at least one ethanolamine phosphate residue, a trimannoside, a glucosamine residue, and an inositol phospholipid. Notwithstanding this core structure a GPI-anchor normally possesses a certain microheterogeniety and therefore a protein having a GPI-anchor normally is a mixture of proteins with homologous GPI-anchors of the same core structure having different side chain modifications.

The term “signal peptide for a GPI-anchor” denotes a C-terminal amino acid sequence of a polyp eptide or protein which consists of one amino acid to which the GPI-anchor will be attached, an optional spacer peptide, and a hydrophobic peptide. Almost all of this signal peptide, i.e. the optional spacer peptide and the hydrophobic peptide, is removed posttranslationally by the enzyme GPI-transaminase and a bond between the amino group of the core ethanolamine phosphate of the GPI-anchor and the amino acid to which the GPI-anchor is attached is formed.

The term “transmembrane domain” as used within this application denotes a polyp eptide or protein which is encoded on the DNA level by at least one exon and which comprises an extracellular, a transmembrane, and an intracellular region. A transmembrane domain generally comprises three distinct structural regions: an N-terminal extracellular region, a central conserved transmembrane stretch, and a C-terminal cytoplasmatic region. In one embodiment the transmembrane domain comprises in N- to C-terminal direction an extracellular region and a transmembrane region. The transmembrane domain may additionally comprise an intracellular or cytoplasmatic region.

The term “alternatively spliceable nucleic acid” denotes a nucleic acid beginning with a 5′ splice donor site and terminated by a 3′ splice acceptor site. This alternatively spliceable nucleic acid comprises a non-coding region which is not constitutively spliced out of the corresponding pre-mRNA, such as, for example, the intron after the exon encoding an immunoglobulin heavy chain C_{H}3 or C_{H}4 domain. The “alternative splicing event” taking place at the 5′ splice donor site of the alternatively spliceable nucleic acid is a decision event whether the alternatively spliceable nucleic acid is spliced out of the pre-mRNA or if it is at least partially maintained and comprised in the mature (processed) mRNA.

The term “alternative splicing” and grammatical equivalents thereof as used herein refers to a process in eukaryotic cells in which from a single pre-mRNA due to different processing of one or more introns different mature mRNAs can be obtained and accordingly different isoforms of a polypeptide can be expressed. In one embodiment of the invention a single, i.e. only one, intron of the produced pre-mRNA can be spliced alternatively. In another embodiment the second nucleic acid can be spliced alternatively. In a further embodiment comprises the second nucleic acid an alternatively spliceable intron. The different processing is a “yes/no” decision, i.e. in the alternative splicing process the intron to be processed, i.e. the “alternatively spliceable nucleic acid”, is either at least partially retained or spliced out. This has not to be understood as a branching point mechanism resulting in different exons to follow. It is in fact a mechanism in which an alternatively spliceable nucleic acid is either spliced out or at least partially maintained in the mature mRNA. With this mechanism the alternatively spliceable nucleic acid and, thus, the therein comprised in frame translational stop codon are either retained or removed.

Alternative splicing is a regulatory mechanism in eukaryotic cells. With alternative splicing different combinations of exons in a mature mRNA can be obtained from the same pre-mRNA giving rise to a plurality of different proteins encoded by the same DNA.

In order to allow alternative splicing the last exon encoding the C-terminal domain of the antibody heavy chain has to be without an in frame translational stop codon.

The term “an in frame translational stop codon” denotes a translational stop codon (TAA, TAG, or TGA) which is succeeding a coding region of a nucleic acid without a frameshift of the reading frame with respect to the preceding coding region of the nucleic acid, i.e. which terminates the coding region during translation. An in frame translational stop codon is operably linked to the preceding coding region of a nucleic acid.

The term “without an in frame translational stop codon” denotes the absence of a translational stop codon (TAA, TAG, or TGA) in the designated nucleic acid and/or the presence of a translational stop codon, which can be found within or at the end of a coding region of a nucleic acid, but that is due to one or two basepair shifts not recognized during the translation of the processed mRNA (i.e. out-of-frame, not operably linked) and thus does not terminate the coding region in the translation process.

A “spliceable nucleic acid” is characterized by at least a 5′ splice donor site, a 3′ splice acceptor site, and a so called branch site, which is normally located 20-50 bases upstream of the acceptor site. This architecture affects the recognition and the excision of the nucleic acid from the 5′ splice donor site to the 3′ splice acceptor site from the pre-mRNA during RNA splicing. During the splicing step the mature mRNA from which a polypeptide or protein is translated is generated. In one embodiment of the present invention at least one nucleic acid, preferably the second nucleic acid, is a spliceable nucleic acid containing additional regulatory elements, such as an in frame stop codon.

But the splicing process is not exclusive. It is, e.g., possible that an intron is not removed during pre-mRNA processing from the pre-mRNA and is, thus, at least partially embedded into the mature mRNA. If an in frame stop codon is present in this “optionally” included intron the translation stops at this stop codon and a variant of the encoded polypeptide is produced.

The recognition and excision of an intron is often regulated by additional cis-acting elements in the pre-mRNA. Due to their function and position these elements are referred to as exonic splice enhancer (ESE), exonic splice silencer (ESS), intronic splice enhancer (ISE), or intronic splice silencer (ISS), respectively (Black, D. L., Annu Rev. Biochem. 72 (2003) 291-336).

The genomic DNA of most eukaryotic genes has an intron-exon-organization. For example, within the exon encoding the C-terminal domain of the secreted form of an immunoglobulin heavy chain (i.e. C_{H}3 or C_{H}4, respectively) is a 5′ splice donor site.

If this splice donor site is not effective in the processing of the heavy chain pre-mRNA, the intron following this exon, which contains a stop codon, is at least partially retained in the mature mRNA. The mRNA is then translated into an immunoglobulin heavy chain that ends with a C_{H}3 or C_{H}4 domain and represents a soluble immunoglobulin. This is the major processing pathway for immunoglobulin heavy chain genes in immunoglobulin secreting cells.

If this splice donor site is effective in the processing of the immunoglobulin heavy chain pre-mRNA, the consecutive intron, and, thus, the stop codon is removed.

Hence the translation does not stop after the C-terminal domain of an immunoglobulin heavy chain. Furthermore, translation is continued with the succeeding spliced to exons encoding a transmembrane domain. This minor processing pathway for immunoglobulin heavy chain genes results in a plasma-membrane-bound immunoglobulin form presented on the cell surface of an immunoglobulin producing cell.

This process is referred to as “alternative splicing” and the nucleic acid (i.e. the intron) optionally removed in this process is referred to as “alternatively spliceable nucleic acid”.

If a nucleic acid encoding a heterologous polypeptide or a protein is linked to a nucleic acid encoding at least a fragment of a transmembrane domain or to a nucleic acid encoding a signal peptide for a GPI-anchor by/via an alternatively spliceable nucleic acid, i.e. an alternatively spliceable nucleic acid is located in between these two nucleic acids, and whereby these three nucleic acids are operably linked, two variants of the heterologous polypeptide or protein are expressed: a soluble variant, i.e. a variant only comprising the polypeptide or protein, and a plasma-membrane-bound variant, i.e. a variant comprising both, the polypeptide or protein and the transmembrane domain or the GPI-anchor.

For example, for the recombinant expression of immunoglobulin heavy chains in eukaryotic cells a nucleic acid either with genomic intron-exon-organization or only containing the coding regions, i.e. cDNA, is employed. In both cases the nucleic acid ends with the stop codon after the exon encoding the C-terminal domain of the immunoglobulin heavy chain. The thereafter in the genomic organization succeeding introns and exons, comprising an alternatively spliceable nucleic acid and a transmembrane domain, are omitted. Therefore with such a nucleic acid only a soluble immunoglobulin heavy chain is obtained.

If for recombinant expression of immunoglobulins or fragments thereof the genomic organization of the immunoglobulin heavy chain gene is retained at least partially, i.e. if the intron after the exon encoding the C-terminal domain (i.e. the alternatively spliceable nucleic acid) and the succeeding exon(s) encoding a transmembrane domain are retained, alternative splicing is possible. In the alternative splicing event the 3′ terminal codons and the stop codon of the CH3- or CH4-domain encoding exon, respectively, are removed as/with the intronic sequence and a different, mature mRNA is generated instead, in which the coding region, i.e. the reading frame, is elongated at its 3′ end by the additionally maintained exon(s). This mRNA is translated into a C-terminally extended immunoglobulin heavy chain which contains an additional transmembrane domain, or a fragment thereof, encoded by the additional 3′ exon(s). This elongated immunoglobulin heavy chain is incorporated during the assembly of immunoglobulins resulting in plasma-membrane-bound immunoglobulins. It has now surprisingly been found that with such a nucleic acid according to the invention transfected cells producing a heterologous polypeptide can be selected. This methodology is generally applicable and is not restricted to immunoglobulins. To practice this methodology the nucleic acid for recombinant expression of a heterologous polypeptide without an in frame stop codon has to be operably linked to and in frame with the alternatively spliceable nucleic acid derived from an immunoglobulin comprising an in frame translational stop codon and a polyadenylation site. The succeeding third nucleic acid is variable as well and can be selected from any nucleic acid encoding a transmembrane domain or a fragment thereof as well as from any nucleic acid encoding a signal peptide for a GPI-anchor. These elements, i.e. the nucleic acid encoding the polypeptide, the alternatively spliceable nucleic acid, and the nucleic acid encoding the transmembrane domain or the signal peptide for a GPI-anchor, can be selected and combined from different genes as well as different organisms. The only prerequisite is that the three nucleic acids are combined in such a way that the translational stop codon in the alternatively spliceable nucleic acid is in frame with the reading frame of the nucleic acid encoding the polypeptide, i.e. it can be recognized by the ribosome and translation is terminated.

Generally speaking, with the alternative splicing optionally a fraction of the C-terminus of the soluble form of the heterologous polypeptide is/may be removed from the pre-mRNA as part of an intron. This fraction encompasses optionally the 3′ terminal codons, the 3′ untranslated region, and the stop codon, of the secreted form. Therefore, the nucleic acid beginning with a 5′ splice donor site and terminated by a 3′ splice acceptor site that is removed optionally overlaps/may overlap with the C-terminus of the not alternatively processed variant.

In one embodiment wherein the first nucleic acid encodes an immunoglobulin heavy chain comprises the first nucleic acid all exons and all but one intron of the genomically organized immunoglobulin heavy chain gene. In one embodiment encodes the third nucleic acid either a fragment of a transmembrane domain or a signal peptide for a GPI-anchor, whereby the fragment of the transmembrane domain is encoded by a single exon. In another embodiment is the transmembrane domain an immunoglobulin transmembrane domain encoded by an M1-M2-exon-fusion, i.e. by a single exon without the genomically intervening intron. In one embodiment the immunoglobulin transmembrane domain is encoded by a cDNA.

By introducing a nucleic acid with an at least partially retained overall genomic organization of an immunoglobulin heavy chain gene into a host cell, a cell is obtained, that expresses on the one hand soluble heterologous polypeptide and on the other hand plasma-membrane-bound heterologous polypeptide. For example, to obtain the two immunoglobulin variants, i.e. to enable alternative splicing, it is not necessary to maintain the entire genomic organization of the immunoglobulin heavy chain gene, i.e. all introns and exons. It is only required to maintain the alternative splice site in a functional from.

A “functional splice site” is a nucleic acid sequence comprising a 5′ splice donor site and a 3′ splice acceptor site, thereby allowing for the excision of the interj acent nucleic acid sequence from the pre-mRNA. The recognition and excision of an intron is often regulated by additional cis-acting elements on the pre-mRNA. Due to their function and position these elements are referred to as exonic splice enhancer (ESE), exonic splice silencer (ESS), intronic splice enhancer (ISE), or intronic splice silencer (ISS), respectively (Black, D. L., Annu Rev. Biochem. 72 (2003) 291-336, which is incorporated by reference herein).

The plasma-membrane-bound variant of a polypeptide is firmly connected to the cell expressing it. Therefore the plasma-membrane-bound variant can be used as a marker to isolate cells that have been successfully transfected with a nucleic acid for the expression of a heterologous polypeptide or protein, e.g. an immunoglobulin. In one embodiment the polypeptide is an immunoglobulin. In one embodiment the immunoglobulin is selected from the group of IgG, IgE, and IgA.

In the next step of the method the pairs of the DNA molecules are cloned into the lentiviral expression vector.

Thereafter the lentiviral expression library is introduced into a first population of mammalian cells. The transduced cells display the antibodies of the lentiviral expression library on their surface. From the library of transduced cells (i.e. from the first population of mammalian cells) one or more cells is/are selected for the capability of the antibody displayed on its/their surface that specifically binds the antigen of interest or a fragment or antigenic determinant thereof.

In one embodiment the antibody that specifically binds an antigen of interest is a humanized or human antibody, especially a human antibody.

In one embodiment the antibody that specifically binds an antigen of interest is a full length antibody.

The antibody displayed on the surface of the mammalian cell is expressed as a full length antibody comprising a transmembrane region.

The antibody secreted into the cultivation medium by the mammalian cell is a full length antibody, i.e. without a transmembrane domain.

In one embodiment each member of the expression library, especially the lentiviral expression library, encodes a full length antibody, wherein the antibody is expressed as secreted antibody and as membrane-bound antibody comprising a transmembrane region.

In one embodiment the variability of antigen-specific antibodies is increased by randomly combining different light and heavy chain variable regions.

The cloning of variable regions is a standard procedure generally known in the art and has been described for various species, including humans, non-human primates, mouse, rabbit, and chicken. For review see Barbas III, et al., (eds.), Phage Display—A Laboratory Manual, Cold Spring Habour Press (2001), in particular the chapter Andris-Widhopf et al., Generation of Antibody Libraries: PCR Amplification and Assembly of Light- and Heavy-chain Coding Sequences, therein. Andris-Widhopf et al. discloses sequences of oligonucleotides capable of amplifying variable region coding regions (VR coding regions), especially HCVR coding regions or LCVR coding regions, of the afore mentioned species. Furthermore, oligonucleotides capable of amplifying HCVR coding regions or LCVR coding regions, especially human HCVR coding regions or LCVR coding regions, can be designed by the artisan by comparing known sequences of antibody coding regions which are available from databases such as, for example, Immuno genetics (http://imgt.cines.fr/), Kab at (www.kabatdatabase.com), and Vbase (http://vbase.mrc-cpe.cam.ac.uk/), and by identifying consensus sequences suitable for primer design. Based on general knowledge in molecular biology, on the afore mentioned manual (Barbas III, et al., (eds.) Phage Display—A Laboratory Manual, Cold Spring Habour Press (2001)) and the references cited therein, the artisan is able to design oligonucleotides capable of amplifying HCVR coding regions or LCVR coding regions, wherein in one embodiment the primer comprise suitable restriction sites for the cloning of the amplified products. Further Strategies for amplifying and cloning VRs are described in Sblattero, D. and Bradbury, A., Immunotechnology 3 (1998) 271-278 and Weitkamp et al., J. Immunol. Meth. 275 (2003) 223-237.

In one embodiment the variable region nucleic acid comprises restriction sites (RS) to allow for cloning of the assembled coding regions in a defined orientation in the lentiviral expression vector. In one embodiment, the restriction sites are distinct from one another and at least one of them generates a single-stranded overhang (“sticky end”), thus allowing for directional cloning. In one embodiment the RS are eight or more base pairs long and recognized by “rare cutting” restriction enzymes selected from but not limited to the list of Ascl, Fsel, Notl, Pacl, Pmel, Sfil and Swal.

The human HCVR, human kappa LCVR and human lambda LCVR coding regions are amplified by PCR with mixtures of specific sense and antisense primers annealing in the framework 1 and 4 regions, respectively. The principal set of primers is described here: Sblattero, D. and Bradbury A., Immunotechnology 3 (1998) 271-278. As an alternative to the use of a specific mix of antisense primers for the amplification of HCVR, kappa LCVR and lambda LCVR coding sequences, one antisense primer annealing in the gamma, kappa and lambda constant region can be used, respectively.

The efficiency of the subsequent cloning of specific variable region (VR) coding regions can be enhanced by pre-amplifying the transcriptome of the sub-population of B-cells, especially by using the template switch protocol as described by Zhu, et al., BioTechniques 30 (2001) 892-897. However, the pre-amplification of the transcriptome needs to be balanced against the possible loss of certain rare cDNA species and the possible accumulation of sequence errors.

In one embodiment, the transcribing of the RNA to cDNA comprises the steps of pre-amplifying the transcriptome of the sub-population of B-cells or the single B-cell or the clonal population of B-cells, wherein the pre-amplifying comprises the steps of:
(a) selectively transcribing polyadenylated mRNA contained in the RNA to single stranded cDNA; and
(b) amplifying double stranded cDNA from the single stranded cDNA.

In one embodiment amplifying the double stranded cDNA is performed using one or more of the oligonucleotides of SEQ ID NO: 1 to 11. In one embodiment the number of PCR cycles is less than 20, less than 15, of from 10 to 14, or about 14.

In one embodiment the pool of DNA molecules, especially the first and/or the second pool of DNA molecules is either generated by pooling DNA molecules obtained in independent PCR reactions.

The mixture of oligonucleotides, the first mixture of oligonucleotides and/or the second mixture of oligonucleotides comprises or consists of exactly one pair of oligonucleotides capable of amplifying VR coding regions, especially HCVR coding regions or LCVR coding regions.

In one embodiment the generating of the pool of DNA molecules, especially the first and/or the second pool of DNA molecules is performed in a single reaction using more than one pair of oligonucleotides in the reaction.

In one embodiment the mixture of oligonucleotides, especially the first mixture of oligonucleotides, comprises at least two oligonucleotides capable of amplifying human HCVR coding regions.

In one embodiment the mixture of oligonucleotides, especially the first mixture of oligonucleotides, comprises at least two, especially all, oligonucleotides selected from the group consisting of SEQ ID NO: 1 to SEQ ID NO: 11.

In one embodiment the mixture of oligonucleotides, especially the second mixture of oligonucleotides, comprises at least two oligonucleotides capable of amplifying kappa LCVR coding regions, especially human LCVR coding regions.

In one embodiment the mixture of oligonucleotides, especially the second mixture of oligonucleotides, comprises at least two oligonucleotides capable of amplifying kappa LCVR coding regions, wherein especially the mixture of oligonucleotides, especially the second mixture of oligonucleotides, comprises at least two, especially all, oligonucleotides selected from the group consisting of SEQ ID NO: 12 to SEQ ID NO: 19.

In one embodiment the mixture of oligonucleotides, especially the second mixture of oligonucleotides, comprises at least two oligonucleotides capable of amplifying lambda LCVR coding regions, especially human lambda LCVR coding regions.

In one embodiment the mixture of oligonucleotides, especially the second mixture of oligonucleotides, comprises at least two oligonucleotides capable of amplifying lambda LCVR coding regions, wherein further especially the mixture of oligonucleotides, especially the second mixture of oligonucleotides, comprises at least two, especially all, oligonucleotides selected from the group consisting of SEQ ID NO: 20 to SEQ ID NO: 28.

In one embodiment the mixture of oligonucleotides, the first mixture of oligonucleotides or the second mixture of oligonucleotides comprise a total amount of primers capable of amplifying VR coding regions, wherein all forward primers and all reverse primers contained in the total amount are in an equimolar ratio.

In one embodiment the antibody encoded by the expression library, especially by the lentiviral expression library, comprises exactly one LCVR.

To ensure cell surface display of the antibody, the antibody chains are expressed with a signal peptide directing the antibody chains to the secretory pathway through the endoplasmic reticulum of the cell, especially of the mammalian cell, wherein especially the signal peptide is located at the N-terminus of each of the antibody chains, and wherein further especially the signal peptide is cleaved off the antibody chains during the processing and transport in the cell, especially in the mammalian cell. Furthermore, the antibody heavy chain is expressed at a certain fraction with a transmembrane region anchoring the antibody in the cell membrane.

Very especially, the transmembrane region is located at the C-terminus of the antibody heavy chain and causes the antibody to remain attached to the outer surface of the cell. The anchoring of the antibody in the cell membrane can also be achieved, for example, by GPI-linking (Moran & Caras, The Journal of Cell Biology 115 (1991) 1595-1600).

Signal peptides directing a protein to the secretory pathway of a eukaryotic cell are generally known in the art and are disclosed, for example, in Nielsen, et al., Protein Engineering 10 (1997) 1-6.

In one embodiment, the signal peptide is derived from a secretory or type I transmembrane protein.

In one embodiment, the signal peptide is derived from a secretory protein such as member of the serum protein family (albumin, transferrin, lipoproteins, immunoglobulins), an extracellular matrix protein (collagen, fibronectin, proteoglycans), a peptide hormone (insulin, glucagon, endorphins, encephalins, ACTH), a digestive enzyme (trypsin, chymotrypsin, amylase, ribonuclease, deoxyribonuclease) or a milk protein (casein, lactalbumin).

In one embodiment, the signal peptide is derived from an immunoglobulin, especially a light chain variable region.

In one embodiment the signal peptide is a mouse Ig kappa light chain signal peptide.

In one embodiment, the transmembrane region is derived from an integral membrane protein.

In one embodiment, the transmembrane region is an internal stop-transfer membrane-anchor sequence derived from a type I transmembrane protein (Do, et al., Cell 85 (1996) 369-78; Mothes, et al., Cell 89 (1997) 523-533) such as a cell adhesion molecule (integrins, mucins, cadherins), a lectin (Sialoadhesin, CD22, CD33), or a receptor tyrosine kinase (insulin receptor, EGF receptor, FGF receptor, PDGF receptor).

In one embodiment the transmembrane region is the transmembrane region of human membrane-bound immunoglobulin of the class G.

In one embodiment, the transmembrane region is derived from a receptor tyrosine kinase, more especially from human platelet-derived growth factor receptor (hPDGFR), most especially from hPDGFR B chain (accession number NP 002600).

In one embodiment the transmembrane region is derived from human PDGFR beta chain.

Lentiviruses can function in a broad range of host cells, including mammalian, avian, amphibian, reptilian and insect cells. Their genome comprises elements capable of directing expression of proteins, including heterologous proteins, encoded by nucleic acids of the viral genome in large amounts.

The expression of structural and non-structural viral proteins is separated, and the structural proteins can be provided either by a packaging cell line or by a helper virus replicon. In one embodiment, the expression library is based on separate lentiviral RNA replicons. In one embodiment one replicon encodes the nonstructural proteins, the other encodes the structural proteins.

In one embodiment the population of isolated B-cells is derived from an animal exhibiting an increased titer of antibodies that specifically binds the antigen of interest. The titer of antibodies binding an antigen of interest in the blood of an animal can be determined by methods generally known in the art, e.g. by ELISA.

In one embodiment the animal is or has been exposed to the antigen of interest or to a fragment or antigenic determinant thereof, wherein especially the exposure is by way of natural exposure, infection with a pathogen or immunization.

In one embodiment the animal is or has been infected by a pathogen, wherein the pathogen comprises the antigen of interest or a fragment or antigenic determinant thereof.

In one embodiment the population of isolated B-cells is derived from an animal immunized with an immunogenic composition, wherein the immunogenic composition comprises or alternatively consists of: (a) the antigen of interest; (b) a fragment of the antigen of interest; and (c) an antigenic determinant of the antigen of interest.

Any immunogenic composition known in the art may be used in the context of the invention, especially compositions generating a strong immune response. Exemplary immunogenic compositions are compositions comprising a virus-like particle (VLP), especially a VLP of a RNA bacteriophage. Immunogenic compositions useful are reported in WO 2006/097530, WO 2006/045796, WO 2006/032674, WO 2006/027300, WO 2005/117963, WO 2006/063974, WO 2004/084939, WO 2004/085635, WO 2005/068639, WO 2005/108425, WO 2005/117983, WO 2005/004907, WO 2004/096272, WO 2004/016282, WO 2004/009124, WO 2003/039225, WO 2004/007538, WO 2003/040164, WO 2003/031466, WO 2004/009116, and WO 2003/024481.

In one embodiment, the immunizing of the animal is performed with an immunogenic composition, wherein the immunogenicity of the immunogenic composition is enhanced by an immunostimulatory substance, especially by an immunostimulatory oligonucleotide, most especially by a non-methylated CpG-containing oligonucleotide as disclosed, for example, in WO 2003/024481, WO 2005/004907 and WO 2004/084940.

In one embodiment the non-methylated CpG-containing oligonucleotide is G10 (SEQ ID NO: 54 of WO 2005/004907).

In one embodiment the immunizing of the animal with the immunogenic composition is performed by administering the immunogenic compositions to the animal at least three times, especially three to six times, in intervals of at least one week, especially in intervals of two weeks up to three months.

In one embodiment the immunizing of the animal is performed by administering at least 100 μg, especially 200 μg to 1000 μg of the immunogenic composition to the animal per single administration.

In one embodiment the immunogenic composition comprises an adjuvant, especially Freund's complete or incomplete adjuvant or alum.

In one embodiment the population of isolated B-cells or the single B-cells or the clonal population of B-cells is derived from a source selected from: (a) blood; (b) secondary lymphoid organs, especially spleen or lymph node; (c) bone marrow; and (d) tissue comprising memory B-cells. In one embodiment the source is blood.

In one embodiment the population of isolated B-cells comprises or especially consists of peripheral blood mononuclear cells (PBMCs).

In one embodiment, the animal is a mammal or a bird.

In one embodiment, the animal is selected from the group consisting of: (a) human; (b) mouse; (c) rabbit; (d) chicken; and (e) rat.

In one embodiment, the animal is a mammal, especially a rat, a mouse, a rabbit, or a human.

In one embodiment the animal is a transgenic mouse or a transgenic rabbit or a human.

The efficiency of the screening for and cloning of antigen specific antibodies can be significantly increased by enriching antigen specific B-cells. Methods for selecting from the population of isolated B-cells a sub-population of B-cells by selecting B-cells for their capability of that specifically binds the antigen of interest are generally known in the art. These methods are based on the interaction of antigen-specific B-cells contained in the population of isolated B-cells with the antigen of interest.

In one embodiment the selecting from the population of isolated B-cells a sub-population of B-cells or a single B-cell comprises the steps of:
(a) contacting the population of isolated B-cells with the antigen of interest or a fragment or antigenic determinant thereof and
(b) selecting B-cells or a single B-cell that specifically binds the antigen of interest or fragment or antigenic determinant thereof

Methods for selecting from the population of isolated B-cells a sub-population of B-cells are the binding of B-cells to an antigen-covered carrier and FACS sorting and as described in WO 2004/102198.

In one embodiment the selecting from the population of isolated B-cells a sub-population of B-cells or a single B-cell comprises the steps of:
(a) coating a carrier with the antigen of interest or fragment or antigenic determinant thereof;
(b) contacting the population of isolated B-cells with the carrier and allowing the B-cells to bind to the carrier via the antigen of interest or fragment or antigenic determinant thereof;
(c) removing unbound B-cells, wherein especially the carrier comprises or further especially consists of beads, wherein still further especially the beads are paramagnetic beads; and
(d) recovering the sub-populations of B-cells or the single B-cell from the paramagnetic beads.

In one embodiment, the selecting from the population of isolated B-cells a sub-population of B-cells or a single B-cell is performed by FACS sorting.

In one embodiment the selecting from the population of isolated B-cells a sub-population of B-cells or a single B-cell comprises the steps of:
(a) contacting the population of isolated B-cells with the antigen of interest or fragment or antigenic determinant thereof, wherein the antigen of interest or fragment or antigenic determinant thereof is labeled with a fluorescence dye; and
(b) separating B-cells bound to the antigen of interest or fragment or antigenic determinant thereof by FACS sorting.

In one embodiment the fluorescence dye is selected from the group consisting of (a) PerCP, allophycocyanin (APC), (b) texas red, (c) rhodamine, (d) Cy3, (e) Cy5, (f) Cy5.5, (f) Cy7, (g) Alexa Fluor Dyes, especially Alexa 647 nm or Alexa 546 nm (h) phycoerythrin (PE), (i) green fluorescent protein (GFP), (j) a tandem dye (e.g. PE-Cy5), and (k) fluorescein isothiocyanate (FITC).

In one embodiment the fluorescence dye is Alexa 647 nm or Alexa 546 nm.

In one embodiment the labeling of a compound, especially of the antigen of interest or fragment or antigenic determinant thereof, with the fluorescence dye is performed by any method known in the art, especially by direct labeling the compound by coupling the fluorescence dye to the compound, wherein the coupling may be effected via a covalent as well as a non-covalent bound. Alternatively, labeling of a compound, especially of the antigen of interest or fragment or antigenic determinant thereof, with the fluorescence dye is performed indirectly by binding to the compound a second compound, especially an antibody, wherein the second compound comprises the fluorescence dye.

The sub-population of B-cells may, besides the capability of the cells of that specifically binds the antigen of interest, be further selected for additional markers which are specific for those types of B-cells expressing immunoglobulins the cloning of which is intended. Alternatively, certain undesired types of B-cells predominantly expressing undesired types of immunoglobulins may be excluded. Additionally, vitality markers such as, for example, PI (propidium iodide) or 7-AAD (7-Amino-actinomycin) may be applied to select for vital cells. Further additionally or alternatively, cell death or apoptosis markers, such as, for example, YO-PRO-1 or Annexin V may be applied to sort out dead or apoptotic cells.

Furthermore, it is advantageous to include in the selecting from the population of isolated B-cells a sub-population of B-cells a positive selection for the presence of a B-cell specific marker, especially for CD19 or B220.

In one embodiment the selecting from the population of isolated B-cells a sub-population of B-cells or a single B-cell comprises the steps of:
(a) contacting the population of isolated B-cells with the antigen of interest or a fragment or antigenic determinant thereof;
(b) selecting a population of B-cells or a single B-cell that specifically binds the antigen of interest or fragment or antigenic determinant thereof; and
(c) selecting the B-cells for at least one additional parameter, wherein especially the selection for the at least one additional parameter is
   (i) a positive selection for a parameter selected from presence of a B-cell specific marker, especially CD19 or B220, and vitality of the B-cells; and/or
   (ii) a negative selection for a parameter selected from: presence of IgM antibodies; presence of IgD antibodies, presence of cell death markers, and presence of apoptosis markers.

In one embodiment the selecting from the population of isolated B-cells a sub-population of B-cells further comprises the step of selecting for class switched B-cells, especially for IgM- and/or IgD-negative B-cells, most especially for IgM- and IgD-negative B-cells.

In one embodiment, the selecting from the population of isolated B-cells a sub-population of B-cells or a single B-cell comprises the steps of:
(a) contacting the population of isolated B-cells with the antigen of interest or fragment or antigenic determinant thereof, wherein the antigen of interest or fragment or antigenic determinant thereof is labeled with a first fluorescence dye, wherein especially the fluorescence dye is Alexa 647 nm, Alexa 488 or Alexa 546 nm;
(b) contacting the cells of the population of isolated B-cells with anti-IgM and/or anti-IgD antibodies, wherein the anti-IgM and/or anti-IgD antibodies are labeled with a second and/or a third fluorescence dye, wherein the second and/or the third fluorescence dye emits fluorescence at a wavelength which is different from the wavelength of the fluorescence emitted by the first fluorescence dye; and
(c) separating a population of B-cells or a single B-cell bound to the antigen of interest or fragment or antigenic determinant thereof but not bound to the anti-IgM and/or not bound to the anti-IgD antibodies by FACS sorting.

For the efficiency of the subsequent screening process it is advantageous though not absolutely essential, that each cell expressing and displaying an antibody on its surface comprises about one, especially exactly one, single antibody species, wherein especially each cell comprises a different antibody species. This is referred to as “one antibody per cell format”.

A one antibody per cell format can be achieved, for example, by using a viral expression library, especially a lentiviral expression library, and by choosing a low ratio of virus particles per number of eukaryotic, especially mammalian cells, when introducing/transducing the expression library, i.e. the virus particles, into the population of the cells used for the display HEK293.

In one embodiment the expression library is a viral expression library, especially an lentiviral expression library, and the introducing the expression library into a first population of eukaryotic, especially mammalian cells is performed by infecting the eukaryotic, especially mammalian cell with the viral expression library, especially with the lentiviral expression library, wherein further especially the infecting is performed at a multiplicity of infection of at most 10, especially at most 1, more especially at most 0.2, and most especially at most 0.1. In one embodiment the multiplicity of infection is about 0.1.

In one embodiment the isolating of the cell is performed by FACS sorting. In one embodiment the isolating of the cell comprises the steps of:
(a) staining the first population of eukaryotic, especially mammalian cells with the antigen of interest or fragment or antigenic determinant thereof, wherein the antigen of interest or fragment or antigenic determinant thereof is labeled with a fluorescence dye; and
(b) separating an individual cell that specifically binds the antigen of interest, or fragment or antigenic determinant thereof, by means of FACS sorting.

In one embodiment the separating an individual cell that specifically binds the antigen of interest, or fragment or antigenic determinant thereof, by means of FACS sorting comprises the step of further selecting the cell at least one additional parameter. In one embodiment the at least one additional parameter is selected from
(i) a positive selection for vitality of the cell; and/or
(ii) a negative selection for a parameter selected from: presence of IgM antibodies; presence of IgD antibodies, presence of cell death markers, and presence of apoptosis markers.

Negative selection may also include negative selection for the binding of one or more, especially one, undesired antigen(s). It is within the skill of the person skilled in the art to optionally include undesired antigen(s), especially in a non-labeled format, in the screen in order to out-compete cells expressing an antibody binding the undesired antigen(s).

In one embodiment the method further comprises the steps of:
(a) cultivating at least one, especially exactly one, of the individual cells in the presence of a second population of eukaryotic cells, especially mammalian cells;
(b) verifying the capability of the second population of eukaryotic, especially mammalian cells of that specifically binds the antigen of interest, or fragment or antigenic determinant thereof.

In one embodiment the first population of eukaryotic, especially mammalian cells and/or, especially and, the second population of eukaryotic, especially mammalian cells comprises or especially consists of cells selected from: (a) BHK 21 cells, especially ATCC CCL-10; (b) Neuro-2a cells; (c) HEK-293T cells, especially ATCC CRL-11268; (d) CHO-K1 cells, especially ATCC CRL-62; and (e) HEK293 cells.

In one embodiment the first population of eukaryotic, especially mammalian cells and/or the second population of eukaryotic, especially mammalian cells comprises or especially consists of CHO-K1 cells, wherein further especially the expression library is an lentiviral expression library.

The individual cell displaying the antibody of interest can be used to clone and to recombinantly express antibodies comprising the variable regions of the antibody displayed on the cell using methods generally known in the art (see for example Weitkamp, et al., J. Immunol. Meth. 275 (2003) 223-237). In principle, it is possible to express the antibodies in any know form (for different forms of antibodies see Hollinger & Hudson, Nature Biotechnology 23 (2005)), especially as IgG, most especially as fully human IgG.

Thus, herein is reported a method of producing an antibody that specifically binds an antigen of interest the method comprising the steps of:
(a) isolating a cell expressing an antibody according to the method as reported herein;
(b) obtaining RNA from the isolated cell;
(c) synthesizing cDNA encoding the antibody from the RNA;

(d) cloning the cDNA into an expression vector;
(e) expressing the antibody in a cell; and
(f) purifying the antibody.

In one embodiment the antibody comprises a LCVR and a HCVR, wherein especially the HCVR and the LCVR, are derived from the same individual cell.

In one embodiment the synthesizing of the cDNA comprises the step of synthesizing single stranded cDNA from the RNA.

In one embodiment the synthesizing of the cDNA further comprises the step of amplifying the cDNA from the single stranded cDNA, wherein especially the amplifying is performed using
i) one of the oligonucleotides of SEQ ID NO: 1 to 4 and the oligonucleotide of SEQ ID NO: 5 as primer, or
ii) one of the oligonucleotides of SEQ ID NO: 6 to 10 and the oligonucleotide of SEQ ID NO: 11 as primer.

In one embodiment the expressing of the fusion product is performed in mammalian cells, especially in CHO-K1 cells and HEK293 cells.

Herein is reported a method of producing an antibody that specifically binds an antigen of interest by expressing the antibody as an immunoglobulin, especially as a species specific immunoglobulin, most especially as a mouse, rat, rabbit, chicken or human immunoglobulin, most especially as a fully human immunoglobulin.

Herein is reported a method of producing an antibody that specifically binds an antigen of interest, the method comprising the steps of:
(a) isolating a cell expressing an antibody according to the method as reported herein;
(b) obtaining RNA from the cell;
(c) synthesizing cDNA form the RNA;
(d) amplifying from the cDNA a DNA encoding variable regions (VRs) of the antibody expressed by the cell;
(e) generating an expression construct comprising the DNA, wherein the expression construct is encoding at least one VR of the antibody expressed by the cell;
(f) expressing the expression construct in a cell.

In one embodiment, the method comprising the steps of:
(a) isolating a cell expressing an antibody according to the method described above;
(b) obtaining RNA from the cell;
(c) synthesizing cDNA form the RNA;
(d) amplifying from the cDNA a first DNA encoding a HCVR of the antibody expressed by the cell;
(e) generating a first expression construct comprising the first DNA, wherein the first expression construct is encoding a heavy chain immunoglobulin comprising a heavy chain constant region (HCCR) and the HCVR;
(f) amplifying from the cDNA a second DNA encoding a LCVR of the antibody expressed by the cell;
(g) generating a second expression construct comprising the second DNA, wherein the second expression construct is encoding a light chain immunoglobulin comprising a light chain constant region (LCCR) and the LCVR;
(h) expressing the first expression construct and the second expression construct in a cell.

In a further preferred embodiment the HCCR, the HCVR, the LCCR and the LCVR are of human origin.

In one embodiment the expression construct, the first expression construct and/or the second expression construct are further encoding a hydrophobic leader sequence, especially a species specific hydrophobic leader sequence, most especially a human hydrophobic leader sequence. In one embodiment the first expression construct is further encoding a human heavy chain hydrophobic leader sequence. In one embodiment the second expression construct is further encoding a human light chain hydrophobic leader sequence, wherein the human light chain hydrophobic leader sequence is selected from the group consisting of (a) human kappa light chain hydrophobic leader sequence; and (b) human lambda light chain hydrophobic leader sequence.

In one embodiment the synthesizing of the cDNA comprises the step of synthesizing single stranded cDNA from the RNA.

In one embodiment the synthesizing of the cDNA further comprises the step of amplifying the cDNA from the single stranded cDNA.

In one embodiment the HCCR is a human HCCR, especially a human HCCR selected from the group consisting of: (a) human gamma 1 HCCR; (b) human gamma 2 HCCR; and (c) human gamma 4 HCCR.

In one embodiment the LCCR is a human LCCR, especially a human LCCR selected from the group consisting of: (a) human kappa LCCR; and (b) human lambda LCCR.

In one embodiment the amplifying of the first DNA is performed with HCVR specific primers.

In one embodiment the amplifying of the second DNA is performed with LCVR specific primers, wherein especially the LCVR specific primers are selected from kappa LCVR specific primers and lambda LCVR specific primers. In one embodiment the LCVR specific primers are kappa LCVR specific primers, wherein especially the kappa LCVR specific primers are a combination of any one selected from SEQ ID NO: 12 to SEQ ID NO: 18 with SEQ ID NO: 19. In one embodiment the LCVR specific primers are lambda LCVR specific primers, wherein especially the lambda LCVR specific primers are a combination of any one selected from SEQ ID NO: 20 to SEQ ID NO: 27 with SEQ ID NO: 28.

In one embodiment the LCCR is a human kappa LCCR and wherein the LCVR is a human kappa LCVR. In one embodiment the LCCR is a human lambda LCCR and wherein the LCVR is a human lambda LCVR.

In principle, immunoglobulins comprising a heavy and a light chain can be recombinantly produced by expressing two different expression vectors in the same cell. Alternatively, expression constructs encoding the light chain and the heavy chain can be cloned into a single expression vector. Thus, in one embodiment the expressing of the first expression construct and of the second expression construct comprises expressing the first expression construct as part of a first expression vector and expressing the second expression construct as part of a second expression vector, wherein the first expression vector and the second expression vector are co-transfected to the cell. In one embodiment the expressing of the first expression construct and of the second expression construct comprises expressing the first expression construct and the second expression construct as part of the same expression vector.

For the expression of species specific, especially human, antibodies expression cassettes are produced encoding HCCRs or LCCRs of the species, especially of humans, and the corresponding leader sequences and comprising a restriction site allowing to insert the corresponding VR coding regions. In one embodiment the generating the first expression construct comprises the step of cloning the first DNA into a first expression cassette, wherein the first expression cassette is encoding the HCCR, and, especially, the HCCR hydrophobic leader sequence. In one embodiment the generating the second expression construct comprises the step of cloning the second DNA into a second expression cassette, wherein the second expression cassette is encoding the LCCR, and, especially, the LCCR hydrophobic leader sequence.

In one embodiment the antibody is expressed in a form selected from: (a) single chain antibody, especially scFv; (b) diabody; (c) Fab fragment; (d) F(ab′)2 fragment; and (e) full length antibody, especially selected from IgG, IgA, IgE, IgM, and IgD. In one embodiment the antibody is a fully human antibody.

In one embodiment the antibody is expressed as a whole antibody of the IgG class, especially as IgG1, IgG2, IgG3, or IgG4; wherein especially the antibody is a human antibody, most especially a fully human antibody.

The expressing of the antibody may be performed in any eukaryotic expression system known in the art. Typically and especially, the expressing of the antibody is performed in eukaryotic cells, wherein further especially the eukaryotic cells are selected from yeast cells, insect cells and mammalian cells. In one embodiment the expressing of the antibody is performed in mammalian cells, wherein especially the mammalian cells are selected from HEK cells, CHO cells, COS cells. Very especially the mammalian cells are CHO cells.

Herein is further reported an expression vector for displaying full length antibodies on the surface of a eukaryotic, especially mammalian cell. The expression vector is in one embodiment a viral expression vector, more especially a lentiviral expression vector. In one embodiment the expression vector comprises DNA elements encoding a signal peptide, a transmembrane region and wherein the expression vector comprises a restriction site allowing the cloning, especially the orientation specific cloning, of DNA molecules encoding antibody variable regions, into the expression vector. In a further preferred embodiment the expression vector comprises the DNA elements and the restriction site in an orientation allowing the expression of a membrane-bound antibody comprising from the N- to the C-terminus the signal peptide, an antibody heavy chain, and a transmembrane region.

Herein is reported an expression library, especially to an expression library expressing full length antibodies, comprising the expression vector as reported herein.

Herein is reported a eukaryotic, especially mammalian cell comprising the expression vector as reported herein or comprising at least one specimen of the expression library as reported herein.

Herein is further reported a method for antibody optimization/maturation. The method is especially suited for screening small to medium size diversity, i.e. more than 400 variants. The method can be performed using mammalian cells, especially HEK293 cells.

By the cellular display methods as reported herein
enables the expression and display of full length antibodies in/on HEK293 cells;
enables an efficient process requiring limited resources;
enables an economic one tube process as all antibody variants are obtained in one tube;
enables antibody maturation; and
enables the generation of inexpensive libraries, which do not depend on a specific mode of generation e.g. PCR from human donors, or with synthetic DNA-oligomers.

The B-cell based antibody generation and screening methods evolved during the recent years provides for a new method to generate and screen designed/engineered/natural, IgG-based antibody libraries in mammalian cells.

Herein is reported a method using designed/engineered/moderate diverse mammalian cellular libraries of full length antibodies with a diversity of 10³ to 10⁶.

A library size of up to 10⁶ variants allows
for the display of 1.000 light and 1.000 heavy chains (resulting in 10⁶ different antibodies);
for shuffling of a single chain and keeping the other constant (resulting in 10⁶ possible variants);
for the maturation of a single CDR (10⁶ variants allow for the randomization of 4 to 5 amino acid positions (19 amino acid long variants per position, all amino acids without Cys: 130.000 variants if 4 amino acid positions are randomized)).

Herein is reported a method using full length IgG expressed in membrane-bound and secreted form.

Herein is reported a method allowing for custom-based/random design of the antibody sequences.

Herein is reported a method that uses FACS-based panning and/or screening of antibody variants.

The methods as reported herein can be used for the assembly of pre-selected human donor-derived antibody light and heavy chain sequences, e.g. human B-cell derived antibody sequences generated by PCR.

In one embodiment the B-cells are derived/obtained/isolated from blood.

The methods as reported herein can be used for the maturation of the antigen binding properties, such as affinity, species cross-reactivity, pH-dependent antigen binding, of an antibody by e.g. light chain shuffling or modification/randomization of individual (single) CDRs.

The methods as reported herein can be used for the assembly of pre-selected human donor-derived antibody light and heavy chain sequences (e.g. human tumor B-cell derived antibody sequences isolated by PCR).

The methods as reported herein can be used for testing and assembly of rationally designed antibody sequences (e.g. catalytic antibody, pro-antibodies). Thus, the methods as reported herein can be used for antibody engineering by introduction of specific sequence features.

The methods as reported herein can be used for the humanization of antibodies, e.g. for the identification of backmutations in cases if the classical CDR grafting approach fails and/or in cases wherein the testing of 1,000s or 10,000s of variants is required/intended.

The methods as reported herein can be used for the optimization of antibody's biophysical and/or biochemical properties (e.g. stability, aggregation tendency).

The methods as reported herein can be used for the optimization of antibody expression and/or secretion.

The CDR encoding nucleic acid or the variable domain encoding nucleic acid or the B-cell used in the methods as reported herein can be obtained from an immunized animal, or an animal that survived a disease, or an animal that is currently having an active disease, or from transgenic animals having a human IgG locus, or from a naïve animal.

The CDR encoding nucleic acids in the variable domains can be all derived from a naturally occurring variable domain (including those obtained after immunization of an animal), or can be mixed between naturally occurring CDRs and synthetic CDRs, or can be solely synthetic CDRs.

For example a library comprising randomized CDR3 encoding nucleic acids can be a library wherein the individual members are diverse in the length of the encoded CDR3 (e.g. of from 4 to 25 amino acid residues in length), wherein the use of stop codons is avoided, wherein the occurrence of cysteine residues is avoided, wherein glycosylation sites are avoided, wherein instable sequence motives are avoided, and/or wherein the four most common amino acid residues for human CDR3 regions for each of the positions are randomized.

The diversity generating module can be diverse in
randomized CDRs, e.g. randomized CDR3 (sequence design reflecting the human CDR3 length, no stop-codons, no cysteines, no N-glycosylation sites, no instable motifs); and/or
designed CDRs, e.g. “rational” sequence variants based on design or existing sequences; and/or
donor CDRs or variable domains (human donor, immunized animal derived).

The diversity module can be generated by PCR or gene synthesis and the cloning into the expression vector can be effected via classical ligation or sequence and ligation independent cloning (SLIC).

In the expression system an antibody of a single specificity is produced in and displayed by a single cell. This can be achieved by using a viral infection with controlled MOI (lentiviral or bacmam) in a transient system or by single recombination events (LoxP, FLP) in a stable system. The expression system shall assure a high level expression of a membrane-bound and/or secreted fully length antibody.

The screening of the library members can be performed by the isolation of hits via panning and/or by FACS selection. The screening of secreted antibody can be performed in the supernatant. The variable domain encoding nucleic acids of the selected clones are isolated by PCR.

Thus, herein are reported methods for an antigen-driven enrichment of (e.g. affinity-improved) binders with FACS and/or bead-based methods.

### Cellular Display Methods as Reported Herein

### Method for the Display of Full Length Antibody Libraries:

The diversity generating modules, such as a DNA library comprising randomized CDR3 encoding nucleic acid sequences of an antibody, are introduced into the lentiviral display vector as reported herein.

The diversity generating module comprising lentiviral display vector and the required helper plasmids are introduced into an expression system for the generation of infective virus particles.

After isolation of the virus-containing supernatant and quantification of the virus load (e.g. by transduction experiments or via RT-PCR) mammalian cells, such as HEK293 cells, are transduced for library generation with the adjusted MOI, to obtain cells displaying the membrane-bound members of a library and at the same time secreting soluble members of a library.

The individual library members are screened based on the membrane-bound members of the library in order to identify and select cells presenting antibody variants having predetermined characteristics, e.g. with respect to affinity, species cross-reactivity, pH-dependent antigen binding.

In the next step the selected cells are deposited as single cells in order to obtain a clonal cell population or are deposited as pool of cells. Thereafter the deposited cells are cultivated to produce the antibody variant. The secreted antibody variant can be used for further characterization, such as a primary screening.

Clones or populations selected in the first screening are cultured for extended periods.

Thereafter the variable domain encoding nucleic acids are isolated.

### Method for the Display of Bispecific Antibody Libraries:

Bispecific antibodies are generally antibody molecules that specifically bind to two different, non-overlapping epitopes on the same antigen or to two epitopes on different antigens.

Different bispecific antibody formats are known.

Exemplary bispecific antibody formats that can be used in the methods as reported herein are
the Crossmab format: full length IgG antibody comprising a first binding site that specifically binds to a first epitope or antigen and a second binding site that specifically binds to a second epitope or antigen, whereby the individual chains are as follows
   light chain 1 (variable light chain domain+light chain kappa constant domain)
   light chain 2 (variable light chain domain+heavy chain CH1 domain)
   heavy chain 1 (variable heavy chain domain+CH1+Hinge+CH2+CH3 with hole mutation)
   heavy chain 2 (variable heavy chain domain+light chain kappa constant domain+Hinge+CH2+CH3 with knob mutation);
the one-armed single chain format: antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain kappa constant domain+G₄S-Linker+variable heavy chain domain+CH1+Hinge+CH2+CH3 with knob mutation)
   heavy chain (variable heavy chain domain+CH1+Hinge+CH2+CH3 with hole mutation);
the two-armed single chain format: antibody comprising a first binding site that specifically binds to a first epitope or antigen and a second binding site that specifically binds to a second epitope or antigen, whereby the individual chains are as follows
   combined light/heavy chain 1 (variable light chain domain+light chain kappa constant domain+G₄S-Linker+variable heavy chain domain+CH1+Hinge+CH2+CH3 with hole mutation)
   combined light/heavy chain 2 (variable light chain domain+light chain kappa constant domain+G₄S-Linker+variable heavy chain domain+CH1+Hinge+CH2+CH3 with knob mutation);
the common light chain bispecific format: antibody comprising a first binding site that specifically binds to a first epitope or antigen and a second binding site that specifically binds to a second epitope or antigen, whereby the individual chains are as follows
   light chain (variable light chain domain+light chain kappa constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation).
the tetravalent scFv format: bispecific tetravalent antibody comprising a first binding site that specifically binds to a first epitope or antigen and a second binding site (scFv) that specifically binds to a second epitope or antigen, whereby the individual chains are as follows
   light chain (variable light chain domain+light chain kappa constant domain)
   combined heavy chain−scFv (variable heavy chain domain+CH1+Hinge+CH2+CH3+G₄S-Linker+scFv).
the tetravalent scFab format: bispecific tetravalent antibody comprising a first binding site that specifically binds to a first epitope or antigen and a second binding site (scFv) that specifically binds to a second epitope or antigen, whereby the individual chains are as follows
   light chain (variable light chain domain+light chain kappa constant domain)
   combined heavy chain−scFab (variable heavy chain domain+CH1+Hinge+CH2+CH3+G₄S-Linker+scFab).

The structure of the elements of the expression cassettes for the above outlined bispecific antibody formats are as follows (in 5′ to 3′ direction, see also FIG. 7):
the Crossmab format:
[5′-LTR+packaging element+RRE]−[hCMV promoter]−[light chain 1 or 2]−[IRES]−[heavy chain 1 or 2]−[WPRE+3′-LTR];
the one-armed single chain format:
1) [5′-LTR+packaging element+RRE]−[hCMV promoter]−[light chain]−[IRES]−[heavy chain]−[WPRE+3′-LTR], and/or
2) [5′-LTR+packaging element+RRE]−[hCMV promoter]−[combined light/heavy chain]−[WPRE+3′-LTR];
the two-armed single chain format:
[5′-LTR+packaging element+RRE]−[hCMV promoter]−[combined light/heavy chain 1 or 2]−[WPRE+3′-LTR];
the common light chain bispecific format:
1) [5′-LTR+packaging element+RRE]−[hCMV promoter]−[heavy chain 1]−[IRES]−[heavy chain 2]−[WPRE+3′-LTR], and/or
2) [hCMV promoter]−[light chain]−[bGH polyA];
the tetravalent scFv format:
1) [5′-LTR+packaging element+RRE]−[hCMV promoter]−[heavy chain]−[IRES]−[light chain]−[WPRE+3′-LTR];
the tetravalent scFab format:

1) [5′-LTR+packaging element+RRE]−[hCMV promoter]−[heavy chain]−[IRES]−[light chain]−[WPRE+3′-LTR].

The workflow for the display of full length antibodies on the surface of eukaryotic cells and the selection of cells is described in the following:

In the first step the required expression vector(s) as reported herein is (are) constructed based on the bispecific antibody format to be displayed.

Thereafter two independent virus particles are generated for each antibody half. Cells are transfected with shuttle vector and helper plasmids to produce replication incompetent but infective viral particles.

For the generation of the display library either
mammalian cells are infected with a low MOI (Multiplicity of infection) with both viral particles encoding the respective antibody parts, or
mammalian cells are infected with a low MOI with virus particles encoding the first antibody half and subsequently the cells are infected with virus particles for the second antibody half, or
mammalian cells stably expressing a common light chain are infected with virus particle encoding two heavy chains.

Thereafter transduced cells expressing bispecific monoclonal antibodies are selected/enriched.

Antigen binding cells expressing bispecific antibodies are sorted (deposited) as single cells or as pools using e.g. FACS.

The sorted cells are cultured and expanded.

Thereafter a functional screening of the secreted antibodies in the cell supernatant of the FACS sorted and cultured cells expressing bispecific antibodies is performed.

Thereafter picking of cells expressing functional bispecific antibodies is performed.

Finally the combined light and heavy chain expression cassettes are cloned by PCR and DNA-sequencing.

For the generation of the expression vector for bispecific antibodies against a single target rabbits or mice are immunized with recombinant target protein or cells recombinantly or naturally expressing the target protein. Spleen cells or peripheral blood cell are collected after immunization and RNA is prepared. Antigen specific B-cells can be enriched by FACS as bulk using fluorescently labeled antigen as selection marker. Thereafter cDNA is generated and variable domains of heavy and light chain are amplified by PCR and ligated into a display vector for full length IgGs as reported herein or into an expression vector for expression and production.

For the generation of common light chain antibodies transgenic rabbits are immunized as described above. The transgenic rabbit has a knocked out rabbit IgM and kappa Ig locus. The knocked out rabbit Ig locus is replaced by a human Ig locus comprising human light and heavy chain genes. The light chain gene is already fully rearranged in the transgenic Ig locus leading to the expression of a single light chain in all B-cells obtained from these rabbits. The heavy chain transgene is not rearranged. The heavy chain is highly divers through random rearrangement of the variable gene with the J- and D-elements and somatic hypermutation and gene conversion (see e.g. WO 2005/007696).

For the generation of virus particles is the shuttle vector (=lentiviral display vector as reported herein) co-transfected into HEK293 cells together with helper plasmids (e.g. transfection with Cellfectin). The virus particle containing supernatant is harvested by centrifugation. The number of infectious virus particles can be tested by transduction of HEK293 cells with aliquots of the virus stock. The number of antibody expressing HEK293 cells is counted by FACS.

For the generation of antibody expressing and displaying HEK293 cells are the cells infected with a low MOI of shuttle vector containing virus. Cells expressing the specific antibodies are selected and sorted as bulk by FACS after labeling with fluorescently label antigen. After isolation antigen specific combinations of light and heavy chains are isolated by PCR as one fragment including the IRES, i.e. the cognate combination of light and heavy chain variable domain is conserved.

For the generation of half-antibody libraries are PCR-DNA fragments (encoding light and heavy chain of the antigen specific antibodies) ligated in knob and in hole expression vectors. For both the knob and the hole constructs virus particles are generated as described before.

For display of bispecific antibodies are generated HEK293 cells expressing both knob and hole based antibody heavy chains by transduction with the different viruses. For sorting/selection soluble, fluorescently label antigen is added to the cells. Cells are washed for several times to select for bivalent binders (slower off-rate of the antigen bound by two antibody arms). Long half-life binders are sorted as single cells by FACS.

For screening of functional bispecific antibodies the secreted antibodies in the supernatant of the cultured HEK293 cells are tested in a functional assay, cell based or non-cell based (e.g. receptor phosphorylation, proliferation, induction of apoptosis). From selected clones light and heavy chains of functionally active antibodies are cloned by PCR from the HEK293 cells.

One aspect as reported herein is a workflow/method for the display of full length antibodies comprising a common light chain on the surface of eukaryotic cells and the selection of cells and thereby the selection of an antibody comprising the following steps:
immunization of an experimental animal, such as a transgenic rabbit,
selection of antigen-specific B-cells (by FACS, bulk sort),
PCR amplification of heavy chain encoding nucleic acid: two separate polymerase chain reactions introducing unique restriction sites to enable directed cloning into the shuttle vector, one for the knob chain using one or more or all of the primer of SEQ ID NO: 6 to SEQ ID NO: 10 and the primer of SEQ ID NO: 11 and one for the hole chain using one or more or all of the primer of SEQ ID NO: 1 to SEQ ID NO: 4 and the primer of SEQ ID NO: 5; ligation: first heavy chain variable domain encoding nucleic acid into hole-locus without transmembrane domain, i.e. of the EV71-IRES and second heavy chain variable domain encoding nucleic acid into knob-locus with a transmembrane domain, i.e. downstream of the EV71-IRES,
virus generation, infection of a mammalian cell stably expressing a common light chain, selection of bispecific antibodies displayed on the surface of the mammalian cell (by off-rate screening), bulk sort of hits (mammalian cell clones) using FACS,
PCR of the complete first heavy chain encoding nucleic acid and the variable domain encoding nucleic acid (which has no TM domain) of the second heavy chain including the EV71-IRES and cloning into second shuttle vector without transmembrane domain, using e.g. primers of SEQ ID NO: 29 and SEQ ID NO: 30,
virus generation, infection of a mammalian cell expressing a common light chain, single cell sort of cells, screening of the bispecific antibody in the supernatant, and selection of bispecific antibody.

One aspect as reported herein is a workflow/method for the display of full length antibodies comprising a common light chain on the surface of eukaryotic cells and the selection of cells and thereby the selection of an antibody comprising the following steps:
immunization of an experimental animal, such as a transgenic rabbit,
selection of antigen-specific B-cells (by FACS, bulk sort),
PCR amplification of heavy chain encoding nucleic acid (two separate polymerase chain reactions introducing unique restriction sites to enable directed cloning into the shuttle vector: ligation: first heavy chain variable domain encoding nucleic acid into hole-locus with transmembrane-domain, i.e. upstream of the EV71-IRES, and second heavy chain variable domain encoding nucleic acid into knob-locus with a transmembrane domain, i.e. downstream of the EV71-IRES,
virus generation, infection of a mammalian cell expressing a common light chain, selection of mammalian cell membrane-displayed bispecific antibodies (by off-rate screening), bulk sort of hits (mammalian cell clones) using FACS,
PCR of the complete first heavy chain encoding nucleic acid and the variable domain of the second heavy chain encoding nucleic acid (2.2 kbp) including the EV71-IRES and cloning into second shuttle vector without transmembrane-domain; removal of the transmembrane-domain of the first heavy chain by restriction cutting and religation of the vector,
virus generation, infection of a mammalian cell expressing a common light chain, single cell sort of cells, screening of the bispecific antibody in the supernatant, selection of bispecific antibody.

One aspect as reported herein is a workflow/method for the display of full length antibodies comprising a common light chain on the surface of eukaryotic cells and the selection of cells and thereby the selection of an antibody comprising the following steps:
immunization of an experimental animal, such as a transgenic rabbit,
selection of antigen-specific B-cells (by FACS, bulk sort),
PCR amplification of heavy chain encoding nucleic acid: two separate polymerase chain reactions introducing unique restriction sites to enable directed cloning into the shuttle vectors; ligation: first heavy chain variable domain encoding nucleic acid into a first shuttle vector into a hole-locus with or without transmembrane domain, and second heavy chain variable domain encoding nucleic acid into a second shuttle vector into a knob-locus with or without a transmembrane domain, but at least one has a transmembrane domain,
virus generation (one for the first shuttle vector and one for the second shuttle vector), sequential infection of a mammalian cell expressing a common light chain with the first virus and the second virus, selection of bispecific antibodies displayed on the surface of the mammalian cell (by off-rate screening), bulk sort of hits (mammalian cell clones) using FACS,
PCR of the heavy chain variable domains encoding nucleic acid and cloning into a third shuttle vector in a bicistronic expression unit without transmembrane domain and EV71-IRES,
virus generation, infection of a mammalian cell expressing a common light chain, single cell sort of cells, screening of the bispecific antibody in the supernatant, and selection of bispecific antibody.

One aspect as reported herein is a workflow/method for the display of full length bispecific antibodies comprising a common light chain on the surface of eukaryotic cells and the selection of such eukaryotic cells and thereby also the selection of a bispecific antibody comprising the following steps:
a first experimental animal, in one embodiment a transgenic mouse or a transgenic rabbit, is immunized with a first antigen of interest, in one embodiment an extracellular receptor domain, wherein the B-cells of the experimental animal express the same light chain,
a second experimental animal, in one embodiment a transgenic mouse or a transgenic rabbit, is immunized with a second antigen of interest, in one embodiment an extracellular receptor domain, wherein the B-cells of the experimental animal express the same light chain,
whereby the first antigen and the second antigen are different,
selecting the B-cells of the first and second immunized experimental animal, in one embodiment by bulk sorting by FACS,
obtaining the heavy chain encoding nucleic acid of each B-cell by individual PCR amplification by two separate/sequential polymerase chain reactions introducing unique restriction sites to enable directed cloning into a shuttle vector/lentiviral expression vector,
ligation of a first heavy chain variable domain encoding nucleic acid into a shuttle vector/lentiviral expression vector upstream of an IRES, in one embodiment the IRES is the EV71-IRES, into a hole- or knob-locus with transmembrane domain, and ligation of a second heavy chain variable domain encoding nucleic acid into a the same shuttle vector/lentiviral expression vector downstream of the IRES into the respective other locus with a transmembrane domain, i.e. if the heavy chain upstream of the IRES has a hole-locus the heavy chain downstream of the IRES has a knob-locus and vice versa, whereby the first heavy chain variable domain binds to a first antigen and the second variable domain binds to a second antigen, whereby the first antigen and the second antigen are different,
virus generation,
infection of a mammalian cell expressing a common light chain with the virus,
selection of cells displaying bispecific antibodies on their surface by FACS of doubly labeled transduced cells,
PCR of the complete first heavy chain encoding nucleic acid and the variable domain of the second heavy chain encoding nucleic acid (2.2 kbp) including the EV71-IRES and cloning into second shuttle vector without transmembrane-domain; removal of the transmembrane-domain of the first heavy chain by restriction cutting and religation of the vector,
virus generation, infection of a mammalian cell expressing a common light chain, single cell sort of cells, screening of the bispecific antibody in the supernatant, and selection of a bispecific antibody.

One aspect as reported herein is a workflow/method for the display of full length bispecific antibodies comprising a common light chain on the surface of eukaryotic cells and the selection of such eukaryotic cells and thereby also the selection of a bispecific antibody comprising the following steps:
an experimental animal, in one embodiment a transgenic mouse or a transgenic rabbit, is immunized with an antigen of interest, in one embodiment an extracellular receptor domain, wherein the B-cells of the experimental animal express the same light chain,
selecting the B-cells of the immunized experimental animal, in one embodiment by bulk sorting by FACS,
obtaining the heavy chain encoding nucleic acid of each B-cell by individual PCR amplification by two separate/sequential polymerase chain reactions introducing unique restriction sites to enable directed cloning into a shuttle vector/lentiviral expression vector,
ligation of the heavy chain variable domain encoding nucleic acid into a shuttle vector/lentiviral expression vector downstream of an IRES, in one embodiment the IRES is the EV71-IRES, into a heavy chain locus with transmembrane domain, wherein the shuttle vector/lentiviral expression vector comprises upstream of the IRES the common light chain encoding nucleic acid,
virus generation,
infection of a mammalian cell expressing a common light chain with the virus,
selection of cells displaying antibodies on their surface by FACS of antigen specific labeled transduced cells, in one embodiment by bulk sorting by FACS,
obtaining the heavy chain encoding nucleic acid of each selected cell by individual PCR amplification by two separate/sequential polymerase chain reactions introducing unique restriction sites to enable directed cloning into a shuttle vector/lentiviral expression vector,
ligation of a first heavy chain variable domain encoding nucleic acid into a shuttle vector/lentiviral expression vector upstream of an IRES, in one embodiment the IRES is the EV71-IRES, into a hole- or knob-locus without transmembrane domain, and ligation of a second heavy chain variable domain encoding nucleic acid into a the same shuttle vector/lentiviral expression vector downstream of the IRES into the respective other locus without a transmembrane domain, i.e. if the heavy chain upstream of the IRES has a hole-locus the heavy chain downstream of the IRES has a knob-locus and vice versa, whereby the first heavy chain variable domain binds to a first antigen and the second variable domain binds to a second antigen, whereby the first antigen and the second antigen can be the same or different,
virus generation,
infection of a mammalian cell expressing a common light chain with the virus,
selection of a cell secreting a bispecific antibody.

In one embodiment of all aspects the experimental animal whose B-cells express the same light chain, i.e. all B-cells of the experimental animal express only a single light chain, is a transgenic experimental animal. In one embodiment the transgenic experimental animal has a knocked out IgM and kappa Ig locus, which is replaced by a human Ig locus comprising human light and heavy chain genes, whereby the light chain gene is fully rearranged in the transgenic Ig locus and the heavy chain transgene is not rearranged. In this embodiment the heavy chain is highly divers through random rearrangement of the variable gene with the J- and D-elements and somatic hypermutation and gene conversion.

In one embodiment of all aspects the common light chain bispecific antibody comprises a first binding site that specifically binds to a first antigen and a second binding site that specifically binds to a second antigen, whereby the individual chains are as follows
light chain (variable light chain domain+light chain kappa constant domain),
first heavy chain (variable heavy chain domain+CH1+Hinge+CH2+CH3 with hole mutation),
second heavy chain (variable heavy chain domain+CH1+Hinge+CH2+CH3 with knob mutation).

In one embodiment of all aspects as reported herein the primer used for the amplification of the heavy chain variable region from B-cells are i) the primer of SEQ ID NO: 1 to SEQ ID NO: 4 in combination with a primer of SEQ ID NO: 5, and/or ii) the primer of SEQ ID NO: 6 to SEQ ID NO: 10 in combination with a primer of SEQ ID NO: 11.

In one embodiment of all aspects as reported herein the primer used for the amplification of the light chain (kappa) variable region from B-cells are the primer of SEQ ID NO: 12 to SEQ ID NO: 18 in combination with a primer of SEQ ID NO: 19.

In one embodiment of all aspects as reported herein the primer used for the amplification of the light chain (lambda) variable region from B-cells are the primer of SEQ ID NO: 20 to SEQ ID NO: 27 in combination with a primer of SEQ ID NO: 28.

In one embodiment of all aspects as reported herein the primer used for the amplification of the heavy chain variable region from HEK cells are the primer of SEQ ID NO: 29 in combination with the primer of SEQ ID NO: 30.

### Display Vector:

In the basic lentiviral expression vector only a limited capacity for the antibody encoding nucleic acid is available as vectors of more than about 9 kb from 5′-LTR to 3′-LTR cannot be packed into a lentiviral virus particle. The region from the 5′-LTR to the CMV-promoter (2.7 kb) and from the WPRE element to the 3′-LTR (1.5 kb) covers 4.2 kb in size (Example 1). A maximum of 4.8 kb can be integrated without decreasing the efficacy of virus generation.

In more detail a maximum of about 4800 bp can be integrated into the basic vector as the virus titer is halved for each 1000 bp over the 4800 bp limit.

It has been found that an expression cassette for the combined expression light and heavy chain, coupling via the EV71-IRES, can be used to shorten the DNA insert. The encoding nucleic acid for the light chain is used in front of the encoding nucleic acid of the heavy chain.

In one embodiment the EV71-IRES has the sequence of SEQ ID NO: 31.

It has been found that for the coupled expression of two encoding nucleic acids the IRES of enterovirus 71 (EV71) is especially suited. IRES elements derived from the encephalomyocarditis virus (EMCV), the mouse Gtx, the human ELF4g have less than 15% of the efficiency of the EV71-IRES element.

The expression of the bicistronic expression element is driven by the human CMV-promoter comprising Intron A. The bicistronic expression element encodes the secreted (i.e. soluble) and membrane-bound form of a full length human or humanized or chimeric or non-human animal derived antibody.

The elements of the integrate have the following sizes depending on the produced antibody:

i) Membrane-Bound Antibody Only:

| | |
|---|---|
| hCMV intron A | 1100 bp (optional) |
| LC encoding nucleic acid (start to stop) | 750 bp (cDNA) |
| EV71-IRES | 650 bp |
| HC encoding nucleic acid (start to stop) | 1450 bp (cDNA) |
| shortened TM domain (M1/M2 fusion) | 215 bp (cDNA) |
| total | 4165 bp (3065 bp) |

ii) Membrane-Bound and Secreted Antibody (1):

| | |
|---|---|
| LC encoding nucleic acid (start to stop) | 750 bp (cDNA) |
| EV71-IRES | 650 bp |
| HC encoding nucleic acid (start to stop) | 1450 bp (cDNA) |
| TM domain (intron 6 + M1/M2 fusion) | 1517 bp (cDNA) |
| total | 4367 bp |

iii) Membrane-Bound and Secreted Antibody (2):

| | |
|---|---|
| LC encoding nucleic acid (start to stop) | 750 bp (cDNA) |
| EV71-IRES | 650 bp |
| HC encoding nucleic acid (start to stop) | 1450 bp (cDNA) |
| TM domain (intron 6 + M1/M2 fusion) | 1517 bp (cDNA) |
| puromycin-resistance | 600 bp |
| total | 4967 bp |

iv) Membrane-Bound and Secreted Antibody without IRES (2):

| | |
|---|---|
| LC encoding nucleic acid (start to stop) | 750 bp (cDNA) |
| bGH polyA signal sequence | 230 bp |
| hCMV-promoter | 600 bp |
| HC encoding nucleic acid (start to stop) | 1450 bp (cDNA) |
| TM domain (intron 6 + M1/M2 fusion) | 1517 bp (cDNA) |
| bGH polyA signal sequence | 230 bp |
| total | 4777 bp |

In one embodiment the bGH polyA signal sequence has the sequence of SEQ ID NO: 32. In one embodiment the hCMV promoter has the sequence of SEQ ID NO: 33.

In one embodiment the intron6+M1/M2 fusion has the sequence of SEQ ID NO: 34.

v) Membrane-Bound Two Heavy Chains (KiH):

| | |
|---|---|
| HC1 encoding nucleic acid (start to stop) | 1450 bp (cDNA) |
| shortened TM domain (M1/M2 fusion) | 215 bp (cDNA) |
| EV71-IRES | 650 bp |
| HC2 encoding nucleic acid (start to stop) | 1450 bp (cDNA) |
| shortened TM domain (M1/M2 fusion) | 215 bp (cDNA) |
| total | 3980 bp |

vi) Single Membrane-Bound Two Heavy Chains (KiH):

| | |
|---|---|
| HC1 encoding nucleic acid (start to stop) | 1450 bp (cDNA) |
| EV71-IRES | 650 bp |
| HC2 encoding nucleic acid (start to stop) | 1450 bp (cDNA) |
| shortened TM domain (M1/M2 fusion) | 215 bp (cDNA) |
| total | 3765 bp |

vii) Membrane-Bound scFv Format:

| | |
|---|---|
| LC encoding nucleic acid (start to stop) | 750 bp (cDNA) |
| EV71-IRES | 650 bp |
| HC encoding nucleic acid (start to stop) | 1400 bp (cDNA) |
| scFv (including linker) | 750 bp |
| shortened TM domain (M1/M2 fusion) | 215 bp (cDNA) |
| total | 3765 bp |

viii) Membrane-Bound scFab Format:

| | |
|---|---|
| LC encoding nucleic acid (start to stop) | 750 bp (cDNA) |
| EV71-IRES | 650 bp |
| HC encoding nucleic acid (start to stop) | 1400 bp (cDNA) |
| scFab (including linker) | 1470 bp |
| shortened TM domain (M1/M2 fusion) | 215 bp (cDNA) |
| total | 4485 bp |

In one embodiment the M1/M2 fusion has the sequence of SEQ ID NO: 35.

It has been found that the EV71-IRES allows for the combined expression of heavy and light chain using a bicistronic expression element (keeping the vector size small).

It has been found that an increased expression (productivity) using the linking of the light chain expression cassette to the heavy chain expression cassette by EV71-IRES element can be obtained:
px5068 without IRES: 100% antibody expression (reference)
Gtx-IRES (synthetic): 3%
EV71-IRES: 80%
ELF4G-IRES: 5%
EMCV-IRES: 11%

It has been found that an alternative splice anchor can be used to drive simultaneous expression of both membrane-bound and secreted antibody form.

Unique restriction sites have been incorporated to allow for the introduction of variable light and heavy chain encoding nucleic acids generated by PCR.

Degenerated primer (with compatible restrictions sites) have been used that are able to amplify all human frameworks.

With the method as reported herein bispecific antibodies can be identified that are combinations of monoclonal antibodies by combination of different binding specificities derived e.g. from different antibodies obtained during an immunization campaign or by affinity maturation or by humanization.

With the method as reported herein a large number of bispecific antibodies can be generated and screened for the identification of synergistic combinations.

Exemplary items of the invention as reported herein are
1. A method of selecting a cell expressing a bispecific antibody comprising the steps of
   (a) generating a population of eukaryotic cells by transduction with a population of lentiviral virus particles, whereby each of the lentiviral virus particles comprises a bicistronic expression cassette, which comprises a first heavy chain variable domain encoding nucleic acid in a hole- or knob-locus upstream of the EV71-IRES and a second heavy chain variable domain encoding nucleic acid in the respective other locus downstream of the EV71-IRES, whereby the first heavy chain variable domain binds to a first antigen and the second variable domain binds to a second antigen, whereby the first antigen and the second antigen can be the same or different, whereby the eukaryotic cell expresses a common light chain, whereby one or both of the heavy chains further comprise a transmembrane domain at their C-terminus, and
   (b) selecting from the population of eukaryotic cells a cell depending on the properties of the displayed membrane-bound full length bispecific antibody.
2. The method according to item 1, characterized in that only the heavy chain downstream of the EV71-IRES comprises a transmembrane domain at its C-terminus.
3. A method of selecting a cell secreting a bispecific antibody comprising the steps of
   (a) generating a population of eukaryotic cells by transduction with a population of lentiviral virus particles, whereby each of the lentiviral virus particles comprises a bicistronic expression cassette encoding a secreted bispecific antibody, which comprises a first heavy chain variable domain encoding nucleic acid in a hole- or knob-locus upstream of the EV71-IRES and a second heavy chain variable domain encoding nucleic acid in the respective other locus downstream of the EV71-IRES, whereby the first heavy chain variable domain binds to a first antigen and the second variable domain binds to a second antigen, whereby the first antigen and the second antigen can be the same or different, whereby the eukaryotic cell expresses a common light chain, and
   (b) selecting from the population of eukaryotic cells a cell depending on the properties of the secreted full length bispecific antibody.
4. The method according to any one of items 1 to 3, characterized in that each cell of the population of eukaryotic cells displays or secretes a single full length bispecific antibody.
5. The method according to any one of items 1 to 4, characterized in comprising as first step one or more of the following steps:
   immunizing a transgenic animal with an antigen of interest, wherein the B-cells of the experimental animal express the same light chain, and/or
   selecting the B-cells of the immunized experimental animal by bulk sorting by FACS, and/or
   obtaining the heavy chain encoding nucleic acid of each B-cell by individual PCR amplification by two separate/sequential polymerase chain reactions introducing unique restriction sites to enable directed cloning into a shuttle vector/lentiviral expression vector.
6. The method according to any one of items 1 to 2 and 4 to 5, characterized in comprising the step:
   performing a PCR of the complete first heavy chain encoding nucleic acid and the variable domain of the second heavy chain encoding nucleic acid (2.2 kbp) including the EV71-IRES and cloning into second shuttle vector without transmembrane-domain, optionally with the removal of the transmembrane-domain of the first heavy chain if present by restriction cutting and religation of the vector.
7. The method according to any one of items 1 to 2 and 4 to 6, characterized in that the bicistronic expression cassettes comprises in 5′- to 3′-direction
   a promoter,
   a first nucleic acid encoding a first full length antibody heavy chain,
   optionally a nucleic acid encoding a transmembrane domain or a GPI-anchor,
   the EV71-IRES,
   a second nucleic acid encoding a second full length antibody heavy chain, and
   a nucleic acid encoding a transmembrane domain or a GPI-anchor.
8. The method according to any one of items 3 to 6, characterized in that the bicistronic expression cassettes comprises in 5′- to 3′-direction
   a promoter,
   a first nucleic acid encoding a first full length antibody heavy chain,
   the EV71-IRES,
   a second nucleic acid encoding a second full length antibody heavy chain.
9. The method according to any one items 1 to 8, characterized in that the antibody is a bivalent, bispecific antibody.
10. The method according to any one of items 1 to 9, characterized in that the antibody specifically binds to two different antigens or to two epitopes on the same antigen.
11. The method according to any one of items 1 to 10, characterized in that the first full length antibody heavy chain comprises a hole mutation and the second antibody heavy chain comprises a knob mutation.
12. The method according to any one of items 1 to 11, characterized in that the first full length antibody light chain comprises as constant domain a CH1 domain and the first full length antibody heavy chain comprises as first constant domain a CL domain, or the second full length antibody light chain comprises as constant domain a CH1 domain and the second full length antibody heavy chain comprises as first constant domain a CL domain.
13. The method according to any one of items 1 to 12, characterized in that the full length antibody comprises a constant region of human origin, especially of human IgG1, IgG2, or IgG4 class.
14. The method according to any one of items 1 to 13, characterized in that the eukaryotic cell is a mammalian cell or a yeast cell.
15. The method according to any one of items 1 to 13, characterized in that the mammalian cell is a CHO cell or a HEK cell.
16. The method according to any one of items 1 to 15, characterized in that the nucleic acid encoding an immunoglobulin heavy chain comprises all exons and all but one intron of the genomically organized immunoglobulin heavy chain gene.
17. The method according to any one of items 1 to 16, characterized in that the transmembrane domain is a fragment of a transmembrane domain or a signal peptide for a GPI-anchor, whereby the fragment of the transmembrane domain is encoded by a single exon.
18. The method according to any one of items 1 to 17, characterized in that the transmembrane domain an immunoglobulin transmembrane domain encoded by an M1-M2-exon-fusion of a single exon without the genomically intervening intron.
19. The method according to any one of items 1 to 18, characterized in that the transmembrane domain is encoded by a cDNA.
20. The method according to any one of items 1 to 19, characterized in that the antibody is a humanized or human antibody, especially a human antibody.
21. A method of selecting a cell expressing an antibody comprising the steps of
   (a) generating a population of eukaryotic cells by transduction with a population of lentiviral virus particles, whereby each cell of the population of cells displays a membrane-bound full length antibody, whereof at least two chains are encoded by a bicistronic expression cassette, and which specifically binds to one or more antigens or one or more epitopes on the same antigen, and
   (b) selecting from the population of eukaryotic cells a cell depending on the properties of the displayed membrane-bound full length antibody,
   whereby each lentiviral virus particle of the population of lentiviral virus particles comprises a bicistronic expression cassette comprising the EV71-IRES for the expression of the membrane-bound antibody.
22. The method according to item 21, characterized in that each bicistronic expression cassette of the lentiviral virus particle of the population of lentiviral virus particles encodes a different variant of a parent antibody, which specifically binds to one or more antigens or one or more epitopes on the same antigen.
23. The method according to any one of items 21 to 22, characterized in that the bicistronic expression cassettes comprises in 5′- to 3′-direction
   a promoter,
   a first nucleic acid encoding a full length antibody light chain,
   the EV71-IRES,
   a second nucleic acid encoding a full length antibody heavy chain,
   a spliceable intron, and
   a nucleic acid encoding a transmembrane domain or a GPI-anchor.
24. The method according to any one of items 21 to 22, characterized in that the bicistronic expression cassettes comprises in 5′- to 3′-direction
   a promoter,
   a first nucleic acid encoding a first full length antibody heavy chain,
   optionally a nucleic acid encoding a transmembrane domain or a GPI-anchor,
   the EV71-IRES,
   a second nucleic acid encoding a second full length antibody heavy chain, and
   a nucleic acid encoding a transmembrane domain or a GPI-anchor.
25. The method according to any one of items 21 to 22, characterized in that the bicistronic expression cassettes comprises in 5′- to 3′-direction
   a promoter,
   a first nucleic acid encoding a full length antibody light chain,
   the EV71-IRES,
   a second nucleic acid encoding a full length antibody heavy chain linked at its C-terminus to a scFv,
   a spliceable intron, and
   a nucleic acid encoding a transmembrane domain or a GPI-anchor.
26. The method according to any one of items 21 to 22, characterized in that the bicistronic expression cassettes comprises in 5′- to 3′-direction
   a promoter,
   a first nucleic acid encoding a full length antibody light chain,
   the EV71-IRES,
   a second nucleic acid encoding a full length antibody heavy chain linked at its C-terminus to a scFab,
   a spliceable intron, and
   a nucleic acid encoding a transmembrane domain or a GPI-anchor.
27. The method according to any one items 21 to 26, characterized in that each cell of the population of eukaryotic cells displays a membrane-bound full length antibody and secretes a full length antibody.
28. The method according to any one of items 21 to 26, characterized in that each cell of the population of eukaryotic cells displays and secretes a single full length antibody.
29. The method according to any one items 21 to 28, characterized in that the antibody specifically binds to an antigen.
30. The method according to any one items 21 to 23, characterized in that the antibody is a bivalent monospecific antibody.
31. The method according to any one items 21 to 22 and 24, characterized in that the antibody is a bivalent, bispecific antibody.
32. The method according to any one items 21 to 22 and 25 to 26, characterized in that the antibody is a tetravalent, bispecific antibody.
33. The method according to any one items 21 to 28 and 31 to 32, characterized in that the antibody specifically binds to two different antigens or to two epitopes on the same antigen.
34. The method according to any one items 31 to 33, characterized in that the first full length antibody heavy chain comprises a hole mutation and the second antibody heavy chain comprises a knob mutation.
35. The method according to any one items 31 to 34, characterized in that the first full length antibody light chain comprises as constant domain a CH1 domain and the first full length antibody heavy chain comprises as first constant domain a CL domain, or the second full length antibody light chain comprises as constant domain a CH1 domain and the second full length antibody heavy chain comprises as first constant domain a CL domain.
36. The method according to any one of items 21 to 35, characterized in that the full length antibody comprises a constant region of human origin, especially of human IgG1, IgG2, or IgG4 class.
37. The method according to any one of items 21 to 36, characterized in that the eukaryotic cell is a mammalian cell or a yeast cell.
38. The method according to any one of items 21 to 36, characterized in that the mammalian cell is a CHO cell or a HEK cell.
39. The method according to any one of items 21 to 38, characterized in that the nucleic acid encoding an immunoglobulin heavy chain comprises all exons and all but one intron of the genomically organized immunoglobulin heavy chain gene.
40. The method according to any one of items 21 to 39, characterized in that the transmembrane domain is a fragment of a transmembrane domain or a signal peptide for a GPI-anchor, whereby the fragment of the transmembrane domain is encoded by a single exon.
41. The method according to any one of items 21 to 40, characterized in that the transmembrane domain an immunoglobulin transmembrane domain encoded by an M1-M2-exon-fusion of a single exon without the genomically intervening intron.
42. The method according to any one of items 21 to 41, characterized in that the transmembrane domain is encoded by a cDNA.
43. The method according to any one of items 21 to 42, characterized in that the antibody is a humanized or human antibody, especially a human antibody.
44. A bicistronic expression cassette, which comprises in 5′- to 3′-direction
   a promoter,
   a first nucleic acid encoding a full length antibody light chain,
   the EV71-IRES,
   a second nucleic acid encoding a full length antibody heavy chain,
   a spliceable intron, and
   a nucleic acid encoding a transmembrane domain or a GPI-anchor.
45. A bicistronic expression cassette, which comprises in 5′- to 3′-direction
   a promoter,
   a first nucleic acid encoding a first full length antibody heavy chain,
   the EV71-IRES,
   a second nucleic acid encoding a second full length antibody heavy chain, and
   a nucleic acid encoding a transmembrane domain or a GPI-anchor.
46. The bicistronic expression cassette according to any one of items 44 to 45, characterized in that the first full length antibody heavy chain comprises a hole mutation and the second antibody heavy chain comprises a knob mutation.
47. The bicistronic expression cassette according to any one of items 44 to 46, characterized in that the first full length antibody light chain comprises as constant domain a CH1 domain and the first full length antibody heavy chain comprises as first constant domain a CL domain, or the second full length antibody light chain comprises as constant domain a CH1 domain and the second full length antibody heavy chain comprises as first constant domain a CL domain.
48. The bicistronic expression cassette according to any one of items 44 to 47, characterized in that the full length antibody comprises a constant region of human origin, especially of human IgG1, IgG2, or IgG4 class.
49. The bicistronic expression cassette according to any one of items 44 to 48, characterized in that the eukaryotic cell is a mammalian cell or a yeast cell.
50. The bicistronic expression cassette according to any one of items 44 to 49, characterized in that the mammalian cell is a CHO cell or a HEK cell.
51. The bicistronic expression cassette according to any one of items 44 to 50, characterized in that the nucleic acid encoding an immunoglobulin heavy chain comprises all exons and all but one intron of the genomically organized immunoglobulin heavy chain gene.
52. The bicistronic expression cassette according to any one of items 44 to 51, characterized in that the transmembrane domain is a fragment of a transmembrane domain or a signal peptide for a GPI-anchor, whereby the fragment of the transmembrane domain is encoded by a single exon.
53. The bicistronic expression cassette according to any one of items 44 to 52, characterized in that the transmembrane domain an immunoglobulin transmembrane domain encoded by an M1-M2-exon-fusion of a single exon without the genomically intervening intron.
54. The bicistronic expression cassette according to any one of items 44 to 53, characterized in that the transmembrane domain is encoded by a cDNA.
55. The bicistronic expression cassette according to any one of items 44 to 54, characterized in that the antibody is a humanized or human antibody, especially a human antibody.
56. A eukaryotic cell comprising a bicistronic expression cassette according to any one of items 44 to 55.
   57. The eukaryotic cell according to item 56, characterized in that the bicistronic expression cassette has been transduced into the cell.
58. The eukaryotic cell according to any one of items 56 to 57, characterized in that the eukaryotic cell is a mammalian cell or a yeast cell.
59. The eukaryotic cell according to item 58, characterized in that the mammalian cell is a CHO cell or a HEK cell.
60. A lentiviral vector comprising a bicistronic expression cassette, which comprises in 5′- to 3′-direction
   a promoter,
   a first nucleic acid encoding a full length antibody light chain,
   the EV71-IRES,
   a second nucleic acid encoding a full length antibody heavy chain,
   a spliceable intron, and
   a nucleic acid encoding a transmembrane domain or a GPI-anchor.
61. A lentiviral vector comprising a bicistronic expression cassette, which comprises in 5′- to 3′-direction
   a promoter,
   a first nucleic acid encoding a first full length antibody heavy chain,
   the EV71-IRES,
   a second nucleic acid encoding a second full length antibody heavy chain, and
   a nucleic acid encoding a transmembrane domain or a GPI-anchor.
62. The lentiviral vector according to any one of items 60 to 61, characterized in that the full length antibody comprises a constant region of human origin, especially of human IgG1, IgG2, or IgG4 class.
63. The lentiviral vector according to any one of items 60 to 62, characterized in that the eukaryotic cell is a mammalian cell or a yeast cell.
64. The lentiviral vector according to any one of items 60 to 63, characterized in that the mammalian cell is a CHO cell or a HEK cell.
65. The lentiviral vector according to any one of items 60 to 64, characterized in that the nucleic acid encoding an immunoglobulin heavy chain comprises all exons and all but one intron of the genomically organized immunoglobulin heavy chain gene.
66. The lentiviral vector according to any one of items 60 to 65, characterized in that the transmembrane domain is a fragment of a transmembrane domain or a signal peptide for a GPI-anchor, whereby the fragment of the transmembrane domain is encoded by a single exon.
67. The lentiviral vector according to any one of items 60 to 66, characterized in that the transmembrane domain an immunoglobulin transmembrane domain encoded by an M1-M2-exon-fusion of a single exon without the genomically intervening intron.
68. The lentiviral vector according to any one of items 60 to 67, characterized in that the transmembrane domain is encoded by a cDNA.
69. The lentiviral vector according to any one of items 60 to 68, characterized in that the antibody is a humanized or human antibody, especially a human antibody.
70. A eukaryotic cell comprising a lentiviral vector according to any one of items 60 to 69.
71. The eukaryotic cell according to item 70, characterized in that the lentiviral vector has been transduced into the cell.
72. The eukaryotic cell according to any one of items 70 to 71, characterized in that the eukaryotic cell is a mammalian cell or a yeast cell.
73. The eukaryotic cell according to item 72, characterized in that the mammalian cell is a CHO cell or a HEK cell.
74. Use of a lentiviral vector according to any one of items 60 to 69 for the generation of a population of eukaryotic cells for displaying or secreting or both of a full length antibody.
75. The use according to item 74, characterized in that the eukaryotic cell is a mammalian cell or a yeast cell.
76. The use according to item 75, characterized in that the mammalian cell is a

CHO cell or a HEK cell.
77. A lentiviral vector library comprising two or more lentiviral particles each comprising an expression vector according to any one of items 60 to 69, wherein the antibodies encoded by each vector differ in at least one amino acid from each other.
78. The lentiviral vector library according to item 77, characterized in that the vector library comprises of from 1,000 to 1,000,000 different expression vectors 79. The lentiviral vector library according to any one of items 77 to 78, characterized in that the antibodies encoded by the vectors of the vector library differ in at least one amino acid residue in one of the CDRs of the antibody.
80. The lentiviral vector library according to item 79, characterized in that the CDR is the heavy chain CDR3.
81. The lentiviral vector library according to any one of items 77 to 80, characterized in that the expression vector library is obtained by randomization of one or more amino acids residues in one or more CDRs of a parent expression vector.
82. The lentiviral vector library according to any one of items 77 to 81, characterized in that the lentiviral expression vector library is obtained by combination of nucleic acids encoding two different half antibodies.
83. The lentiviral vector library according to any one of items 77 to 82, characterized in that the diversity of the lentiviral vector library is generated by using the HCVR and LCVR encoding nucleic acids obtained from a pool of B-cells producing an antibody that specifically binds to one antigen, or two different antigens, or that specifically binds to two different, non-overlapping epitopes of the same antigen.
84. The lentiviral vector library according to any one of items 77 to 82, characterized in that the diversity of the lentiviral vector library is generated by using pairs of HCVR and LCVR encoding nucleic acids selected from pools of HCVR and LCVR encoding nucleic acids, which are obtained by randomizing at least one codon of the HCVR and the LCVR encoding nucleic acid obtained from a single B-cell producing an antibody that specifically binds to one antigen, or two different antigens, or that specifically binds to two different, non-overlapping epitopes of the same antigen.
85. The lentiviral vector library according to any one of items 77 to 82, characterized in that the diversity of the lentiviral vector library is generated by using pairs of different HCVR encoding nucleic acids and a single LCVR encoding nucleic acid, whereby the different HCVR encoding nucleic acids are obtained by randomizing at least one codon of the HCVR encoding nucleic acid obtained from a single B-cell producing an antibody that specifically binds to one antigen, or two different antigens, or that specifically binds to two different, non-overlapping epitopes of the same antigen.
86. The lentiviral vector library according to any one of items 77 to 82, characterized in that the diversity of the lentiviral vector library is generated by using pairs of different LCVR encoding nucleic acids and a single HCVR encoding nucleic acid, whereby the different LCVR encoding nucleic acids are obtained by randomizing at least one codon of the LCVR encoding nucleic acid obtained from a single B-cell producing an antibody that specifically binds to one antigen, or two different antigens, or that specifically binds to two different, non-overlapping epitopes of the same antigen.
87. The lentiviral vector library according to any one of items 77 to 86, characterized in that the single B-cell is a clonal population of B-cells.
88. The lentiviral vector library according to any one of items 77 to 87, characterized in that the generating of the diversity of the lentiviral expression library comprises the steps of
   (a):
      (i) isolating RNA from the sub-population of B-cells,
      (ii) transcribing the RNA to cDNA;
      (iii) amplifying from the cDNA a first pool of DNA molecules using a first mixture of oligonucleotides comprising at least two oligonucleotides capable of amplifying HCVR coding regions;
      (iv) amplifying from the cDNA a second pool of DNA molecules using a second mixture of oligonucleotides comprising at least two oligonucleotides capable of amplifying LCVR coding regions; and
      (v) providing pairs of one member of the first pool of DNA molecules and one member of the second pool of DNA molecules;
   or (b):
      (i) isolating RNA from a single B-cell, or from a clonal population of B-cells,
      (ii) transcribing the RNA to cDNA;
      (iii) amplifying from the cDNA a first DNA molecule using a first mixture of oligonucleotides comprising at least two oligonucleotides capable of amplifying HCVR coding regions;
      (iv) amplifying from the cDNA a second DNA molecule using a second mixture of oligonucleotides comprising at least two oligonucleotides capable of amplifying LCVR coding regions;
      (v) generating a first pool of DNA molecules by randomizing at least one codon of the first DNA molecule,
      (vi) generating a second pool of DNA molecules by randomizing at least one codon of the second DNA molecule, and
      (vii) providing pairs of one member of the first pool of DNA molecules and one member of the second pool of DNA molecules;
   or (c):
      (i) isolating RNA from a single B-cell, or from a clonal population of B-cells,
      (ii) transcribing the RNA to cDNA;
      (iii) amplifying from the cDNA a first DNA molecule using a first mixture of oligonucleotides comprising at least two oligonucleotides capable of amplifying HCVR coding regions;
      (iv) amplifying from the cDNA a second DNA molecule using a second mixture of oligonucleotides comprising at least two oligonucleotides capable of amplifying LCVR coding regions;
      (v) generating a pool of DNA molecules by randomizing at least one codon of the first DNA molecule, and
      (vi) providing pairs of one member of the pool of DNA molecules and the second DNA molecule;
   or (d):
      (i) isolating RNA from a single B-cell, or from a clonal population of B-cells,
      (ii) transcribing the RNA to cDNA;
      (iii) amplifying from the cDNA a first DNA molecule using a first mixture of oligonucleotides comprising at least two oligonucleotides capable of amplifying HCVR coding regions;
      (iv) amplifying from the cDNA a second DNA molecule using a second mixture of oligonucleotides comprising at least two oligonucleotides capable of amplifying LCVR coding regions;
      (v) generating a pool of DNA molecules by randomizing at least one codon of the second DNA molecule, and
      (vi) providing pairs of one member of the pool of DNA molecules and the first DNA molecule.
89. The lentiviral vector library according to any one of items 77 to 88, characterized in that the nucleic acid encoding an immunoglobulin heavy chain comprises all exons and all but one intron of the genomically organized immunoglobulin heavy chain gene.
90. The lentiviral vector library according to any one of items 77 to 89, characterized in that the transmembrane domain is a fragment of a transmembrane domain or a signal peptide for a GPI-anchor, whereby the fragment of the transmembrane domain is encoded by a single exon.
91. The lentiviral vector library according to any one of items 77 to 90, characterized in that the transmembrane domain an immunoglobulin transmembrane domain encoded by an M1-M2-exon-fusion of a single exon without the genomically intervening intron.
92. The lentiviral vector library according to any one of items 77 to 91, characterized in that the transmembrane domain is encoded by a cDNA.
93. A eukaryotic cell library comprising two or more eukaryotic cells each comprising a bicistronic expression cassette according to any one of items 44 to 55 or a lentiviral vector according to any one of items 60 to 69, wherein the antibodies expressed by each cell differ in at least one amino acid from each other.
94. A eukaryotic cell library comprising the lentiviral vector library according to items 77 to 92.
95. The eukaryotic cell library according to item 94, characterized in that each eukaryotic cell of the eukaryotic cell library expresses a single antibody.
96. The eukaryotic cell library according to any one of items 94 to 95, characterized in that each eukaryotic cell of the eukaryotic cell library displays a single antibody.
97. The eukaryotic cell library according to any one of items 94 to 96, characterized in that the eukaryotic cell library is a population of eukaryotic cells expressing a library of antibodies wherein the encoding nucleic acids are derived from a population of B-cells of an immunized animal.
98. The eukaryotic cell library according to item 97, characterized in that the B-cells are pre-selected for their specificity towards the one or more antigens of interest.
99. The eukaryotic cell library according to any one of items 94 to 98, characterized in that the eukaryotic cell library is a population of eukaryotic cells wherein each cell comprises a first expression cassette encoding a full length antibody that specifically binds to a first antigen and a second expression cassette encoding a full length antibody that specifically binds to a second antigen.
100. The eukaryotic cell library according to any one of items 94 to 99, characterized in that the eukaryotic cell library is a population of eukaryotic cells wherein each cell comprises a first expression cassette encoding a first full length antibody light chain and a first full length antibody heavy chain binding to a first antigen and a second expression cassette encoding a second full length antibody light chain and a second full length antibody heavy chain that specifically binds to a second antigen.
101. The eukaryotic cell library according to any one of items 94 to 100, characterized in that the eukaryotic cell library is a population of eukaryotic cells wherein each cell comprises a expression cassette encoding a first full length antibody heavy chain that specifically binds to a first antigen and a second full length antibody heavy chain that specifically binds to a second antigen, wherein the eukaryotic cell expresses a common light chain.
102. The eukaryotic cell library according to any one of items 94 to 102, characterized in that the first full length antibody heavy chain comprises a hole mutation and the second antibody heavy chain comprises a knob mutation.
103. The eukaryotic cell library according to any one of items 94 to 101, characterized in that the first full length antibody light chain comprises as constant domain a CH1 domain and the first full length antibody heavy chain comprises as first constant domain a CL domain, or the second full length antibody light chain comprises as constant domain a CH1 domain and the second full length antibody heavy chain comprises as first constant domain a CL domain.
104. The eukaryotic cell library according to any one of items 94 to 103, characterized in that the full length antibody comprises a constant region of human origin, especially of human IgG1, IgG2, or IgG4 class.
105. The eukaryotic cell library according to any one of items 94 to 104, characterized in that the eukaryotic cell is a mammalian cell or a yeast cell.
106. The eukaryotic cell library according to any one of items 94 to 105, characterized in that the mammalian cell is a CHO cell or a HEK cell.
107. The eukaryotic cell library according to any one of items 94 to 106, characterized in that the population of isolated B-cells or the single B-cells or the clonal population of B-cells is derived from a source selected from: (a) blood; (b) secondary lymphoid organs, especially spleen or lymph node; (c) bone marrow; and (d) tissue comprising memory B-cells.
108. The eukaryotic cell library according to item 107, characterized in that the population of isolated B-cells comprises or especially consists of peripheral blood mononuclear cells (PBMCs).
109. The eukaryotic cell library according to any one of items 94 to 108, characterized in that the animal is a mammal, especially a rat, a mouse, a rabbit, or a human.
110 The eukaryotic cell library according to item 109, characterized in that the animal is a transgenic mouse or a transgenic rabbit or a human.
111. The eukaryotic cell library according to any one of items 94 to 110, characterized in that the selecting from the population of isolated B-cells a sub-population of B-cells or a single B-cell comprises the steps of:
   (a) contacting the population of isolated B-cells with the antigen of interest or a fragment or antigenic determinant thereof; and
   (b) selecting B-cells or a single B-cell that specifically binds the antigen of interest or fragment or antigenic determinant thereof
112. The eukaryotic cell library according to any one of items 94 to 111, characterized in that the selecting from the population of isolated B-cells a sub population of B-cells or a single B-cell comprises the steps of:
   (a) coating a carrier with the antigen of interest or fragment or antigenic determinant thereof;
   (b) contacting the population of isolated B-cells with the carrier and allowing the B-cells to bind to the carrier via the antigen of interest or fragment or antigenic determinant thereof;
   (c) removing unbound B-cells, wherein especially the carrier comprises or further especially consists of beads, wherein still further especially the beads are paramagnetic beads; and
   (d) recovering the sub-populations of B-cells or the single B-cell from the paramagnetic beads.
113. The eukaryotic cell library according to any one of items 94 to 112, characterized in that the selecting from the population of isolated B-cells a sub population of B-cells or a single B-cell is performed by FACS sorting.
114. The eukaryotic cell library according to any one of items 94 to 113, characterized in that the selecting from the population of isolated B-cells a sub population of B-cells or a single B-cell comprises the steps of:
   (a) contacting the population of isolated B-cells with the antigen of interest or fragment or antigenic determinant thereof, wherein the antigen of interest or fragment or antigenic determinant thereof is labeled with a fluorescence dye; and
   (b) separating B-cells bound to the antigen of interest or fragment or antigenic determinant thereof by FACS sorting.
115. The eukaryotic cell library according to any one of items 94 to 114, characterized in that the selecting from the population of isolated B-cells a sub population of B-cells or a single B-cell comprises the steps of:
   (a) contacting the population of isolated B-cells with the antigen of interest or a fragment or antigenic determinant thereof;
   (b) selecting a population of B-cells or a single B-cell that specifically binds the antigen of interest or fragment or antigenic determinant thereof; and
   (c) selecting the B-cells for at least one additional parameter, wherein especially the selection for the at least one additional parameter is
      (i) a positive selection for a parameter selected from presence of a B cell specific marker, especially CD19 or B220, and vitality of the B-cells; and/or
      (ii) a negative selection for a parameter selected from: presence of IgM antibodies; presence of IgD antibodies, presence of cell death markers, and presence of apoptosis markers.
116. The eukaryotic cell library according to any one of items 94 to 115, characterized in that the selecting from the population of isolated B-cells a sub population of B-cells further comprises the step of selecting for class switched B-cells, especially for IgM- and/or IgD-negative B-cells, most especially for IgM- and IgD-negative B-cells.
117. The eukaryotic cell library according to any one of items 94 to 116, characterized in that the selecting from the population of isolated B-cells a sub population of B-cells or a single B-cell comprises the steps of:
   (a) contacting the population of isolated B-cells with the antigen of interest or fragment or antigenic determinant thereof, wherein the antigen of interest or fragment or antigenic determinant thereof is labeled with a first fluorescence dye, wherein especially the fluorescence dye is Alexa 647 nm, Alexa 488 or Alexa 546 nm;
   (b) contacting the cells of the population of isolated B-cells with anti-IgM and/or anti-IgD antibodies, wherein the anti-IgM and/or anti-IgD antibodies are labeled with a second and/or a third fluorescence dye, wherein the second and/or the third fluorescence dye emits fluorescence at a wavelength which is different from the wavelength of the fluorescence emitted by the first fluorescence dye; and
   (c) separating a population of B-cells or a single B-cell bound to the antigen of interest or fragment or antigenic determinant thereof but not bound to the anti-IgM and/or not bound to the anti-IgD antibodies by FACS sorting.
118. The eukaryotic cell library according to any one of items 94 to 117, characterized in that the library is a viral library, especially an lentiviral library, and the introducing the library into a first population of eukaryotic, especially mammalian cells is performed by infecting the eukaryotic, especially mammalian cell with the viral library, especially with the lentiviral library, wherein further especially the infecting is performed at a multiplicity of infection of at most 10, especially at most 1, more especially at most 0.2, and most especially at most 0.1.
119. The eukaryotic cell library according to item 118, characterized in that the multiplicity of infection is about 0.1.
120. The eukaryotic cell library according to any one of items 94 to 119, characterized in that the isolating of the cell is performed by FACS sorting. In one embodiment the isolating of the cell comprises the steps of:
   (a) staining the first population of eukaryotic, especially mammalian cells with the antigen of interest or fragment or antigenic determinant thereof, wherein the antigen of interest or fragment or antigenic determinant thereof is labeled with a fluorescence dye; and
   (b) separating an individual cell that specifically binds the antigen of interest, or fragment or antigenic determinant thereof, by means of FACS sorting.
121. The eukaryotic cell library according to any one of items 94 to 120, characterized in that the separating an individual cell that specifically binds the antigen of interest, or fragment or antigenic determinant thereof, by means of FACS sorting comprises the step of further selecting the cell at least one additional parameter.
122. The eukaryotic cell library according to any one of items 94 to 122, characterized in that the method further comprises the steps of:
   (a) cultivating at least one, especially exactly one, of the individual cells in the presence of a second population of eukaryotic cells, especially mammalian cells;
   (b) verifying the capability of the second population of eukaryotic, especially mammalian cells of that specifically binds the antigen of interest, or fragment or antigenic determinant thereof.
123. The eukaryotic cell library according to any one of items 94 to 122, characterized in that the first population of eukaryotic, especially mammalian cells and/or, especially and, the second population of eukaryotic, especially mammalian cells comprises or especially consists of cells selected from: (a) BHK 21 cells, especially ATCC CCL-10; (b) Neuro-2a cells; (c) HEK-293T cells, especially ATCC CRL-11268; (d) CHO-Kl cells, especially ATCC CRL-62; and (e) HEK293 cells.
124. The eukaryotic cell library according to any one of items 94 to 123, characterized in that the first population of eukaryotic, especially mammalian cells and/or the second population of eukaryotic, especially mammalian cells comprises or especially consists of CHO-Kl cells, wherein further especially the expression library is an lentiviral expression library.
125. A method of selecting a cell expressing an antibody which specifically binds to an antigen of interest comprising the steps of
   (a) optionally selecting from a population of B-cells a sub-population of B-cells or a single B-cell or a clonal population of B-cells secreting an antibody that specifically binds one or more antigens,
   (b) generating a lentiviral expression library, wherein each member of the lentiviral expression library encodes a variant of the antibody or the antibodies that specifically binds one or more antigens, by
      (i) generating a multitude of DNA molecules, wherein the generating comprises the step of amplifying a pool of DNA molecules from the sub-population of B-cells or the step of generating a library of DNA molecules from the DNA encoding a single antibody that specifically binds to one or two antigens of interest by randomization of the encoding nucleic acid sequence, and
      (ii) cloning the multitude of DNA molecules into a lentiviral expression vector comprising an EV71-IRES linked bicistronic expression cassette for the expression of a full length antibody light chain and a full length antibody heavy chain in a soluble as well as membrane bound form;
   (c) transducing a population of eukaryotic cells with a population of lentiviral virus particles each comprising a member of the lentiviral expression library;
   (d) displaying antibodies encoded by the lentiviral expression library on the surface of the eukaryotic mammalian cells; and
   (e) isolating from the population of eukaryotic cells a cell, wherein the cell is selected for the capability of the antibody displayed on its surface to specifically bind the antigen or antigens of interest or a fragment or antigenic determinant thereof.
126. A method of selecting a cell expressing an bispecific antibody (which specifically binds to two antigens of interest) comprising the steps of
   (a) generating a lentiviral expression library, wherein each member of the lentiviral expression library encodes a variant of the bispecific antibody, by
      (i) generating a multitude of DNA molecules from the DNA encoding a single bispecific antibody by randomization of the encoding nucleic acid sequence, and
      (ii) cloning the multitude of DNA molecules into a lentiviral expression vector comprising an EV71-IRES linked bicistronic expression cassette for the expression of a full length bispecific antibody as membrane bound form;
   (b) transducing a population of eukaryotic cells with a population of lentiviral virus particles each comprising a member of the lentiviral expression library;
   (c) displaying antibodies encoded by the lentiviral expression library on the surface of the eukaryotic mammalian cells; and
   (d) isolating from the population of eukaryotic cells a cell, wherein the cell is selected for the capability of the antibody displayed on its surface to specifically bind the antigens of interest or a fragment or antigenic determinant thereof
127. The method according to any one of items 125 to 126, characterized in that the method comprises generating a multitude of DNA molecules encoding antibodies, the generating a multitude of DNA molecules comprising the steps of:
   (1) amplifying from a sub-population of B-cells a first pool of DNA molecules encoding heavy chain variable regions (HCVRs); and
   (2) amplifying from the sub-population of B-cells a second pool of DNA molecules encoding light chain variable regions (LCVRs);
   (3) cloning a combination of the multitude of DNA molecule encoding LCVRs and of the multitude of DNA molecules encoding HCVRs into a lentiviral expression vector comprising an EV71-IRES linked bicistronic expression cassette for the expression of a full length antibody light chain and a full length antibody heavy chain in a soluble as well as membrane bound form.
128. The method according to any one of items 125 to 127, characterized in that the method comprises generating a multitude of DNA molecules encoding antibodies that specifically binds to one or two antigens of interest, the generating a multitude of DNA molecules comprising the steps of:
   (1) amplifying from a single B-cell or a clonal population of B-cells a DNA molecule encoding the HCVR and a DNA molecule encoding the LCVR, and
   (2) randomization the DNA molecule encoding the HCVR and/or the DNA molecule encoding the LCVR by randomizing at least one codon and thereby generating a multitude of DNA molecules encoding the HCVR and a multitude of DNA molecules encoding the LCVR;
   (3) cloning a combination of the randomized multitude of DNA molecule encoding LCVRs and of the multitude of DNA molecules encoding HCVRs into a lentiviral expression vector comprising an EV71-IRES linked bicistronic expression cassette for the expression of a full length antibody light chain and a full length antibody heavy chain in a soluble as well as membrane bound form.
129. The method according to any one of items 125 to 128, characterized in that the method comprises the generating of a lentiviral expression library, the generating comprising the steps of:
   (i) generating a multitude of DNA molecules encoding antibodies, the generating comprising the steps of:
      (1) isolating mRNA from the sub-population of B-cells;
      (2) transcribing the mRNA to cDNA;
      (3) amplifying from the cDNA a first pool of DNA molecules using a first mixture of oligonucleotides comprising at least two oligonucleotides capable of amplifying HCVR coding regions; and
      (4) amplifying from the cDNA a second pool of DNA molecules using a second mixture of oligonucleotides comprising at least two oligonucleotides capable of amplifying LCVR coding regions;
      (ii) cloning a pair of DNA molecules of the first and the second pool of DNA molecules into a lentiviral expression vector comprising an EV71-IRES linked bicistronic expression cassette for the expression of a full length antibody light chain and a full length antibody heavy chain in a soluble as well as membrane bound form.
130. The method according to any one of items 135 to 129, characterized in that the method comprises the generating of a lentiviral expression library, the generating comprising the steps of:
   (i) generating a multitude of DNA molecules encoding antibodies that specifically binds to one or two antigens, the generating comprising the steps of:
      (1) isolating mRNA from a single B-cell or a clonal population of B-cells;
      (2) transcribing the mRNA to cDNA;
      (3) amplifying from the cDNA a first DNA molecule using a first mixture of oligonucleotides comprising at least two oligonucleotides capable of amplifying HCVR coding region; and
      (4) amplifying from the cDNA a second DNA molecule using a second mixture of oligonucleotides comprising at least two oligonucleotides capable of amplifying LCVR coding region;
      (5) randomizing the first and/or the second DNA molecule and thereby generating a first pool of DNA molecules and a second pool of DNA molecules,
   (ii) cloning a pair of DNA molecules of the first and the second pool of DNA molecules into a lentiviral expression vector comprising an EV71-IRES linked bicistronic expression cassette for the expression of a full length antibody light chain and a full length antibody heavy chain in a soluble as well as membrane bound form.
131. The method according to any one of items 125 to 130, characterized in that the eukaryotic cell is a mammalian cell or a yeast cell.
132. The method according to item 131, characterized in that the mammalian cell is a CHO cell or a HEK cell.
133. The method according to any one of items 125 to 132, characterized in that the animal is selected from the group consisting of sheep, elk, deer, donkey, mule deer, mink, horse, cattle, pig, goat, dog, cat, rat, hamster, guinea pig, and mouse. In one embodiment the animal is a mouse, a rat or, a primate.
134. The method according to any one of items 125 to 133, characterized in that the animal is a non-human primate or a human.
135. The method according to any one of items 125 to 134, characterized in that the animal is a transgenic animal with a human immunoglobulin locus.
136. The method according to any one of items 125 to 135, characterized in that the nucleic acid is obtained by selecting from a population of isolated B-cells a sub-population of B-cells by selecting B-cells for their capability of that specifically binds the antigen of interest.
137. The method according to any one of items 125 to 136, characterized in that the nucleic acid is obtained by selecting from a population of isolated B-cells a single B-cell by selecting a B-cell for its capability of that specifically binds the one or two antigens of interest.
138. The method according to any one of items 125 to 137, characterized in that the single B-cell is a clonal B-cell population.
139. The method according to any one of items 125 to 138, characterized in that the nucleic acid is obtained by amplifying the variable domain encoding nucleic acid from the isolated mRNA of a single B-cell or a clonal B-cell population and transcribing the amplified mRNA into cDNA.
140. The method according to any one of items 125 to 139, characterized in that the diversity of the lentiviral vector library is generated by using the HCVR and LCVR encoding nucleic acids obtained from a pool of B-cells producing an antibody that specifically binds to one antigen, or two different antigens, or that specifically binds to two different, non-overlapping epitopes of the same antigen.
141. The method according to any one of items 125 to 140, characterized in that the diversity of the lentiviral vector library is generated by using pairs of HCVR and LCVR encoding nucleic acids selected from pools of HCVR and LCVR encoding nucleic acids, which are obtained by randomizing at least one codon of the HCVR and the LCVR encoding nucleic acid obtained from a single B-cell producing an antibody that specifically binds to one antigen, or two different antigens, or that specifically binds to two different, non-overlapping epitopes of the same antigen.
142. The method according to any one of items 125 to 140, characterized in that the diversity of the lentiviral vector library is generated by using pairs of different HCVR encoding nucleic acids and a single LCVR encoding nucleic acid, whereby the different HCVR encoding nucleic acids are obtained by randomizing at least one codon of the HCVR encoding nucleic acid obtained from a single B-cell producing an antibody that specifically binds to one antigen, or two different antigens, or that specifically binds to two different, non-overlapping epitopes of the same antigen.
143. The method according to any one of items 125 to 142, characterized in that the diversity of the lentiviral vector library is generated by using pairs of different LCVR encoding nucleic acids and a single HCVR encoding nucleic acid, whereby the different LCVR encoding nucleic acids are obtained by randomizing at least one codon of the LCVR encoding nucleic acid obtained from a single B-cell producing an antibody that specifically binds to one antigen, or two different antigens, or that specifically binds to two different, non-overlapping epitopes of the same antigen.
144. The method according to any one of items 125 to 143, characterized in that the single B-cell is a clonal population of B-cells.
145. The method according to any one of items 125 to 144, characterized in that the generating of the diversity of the lentiviral expression library comprises the steps of
   (a):
      (i) isolating RNA from the sub-population of B-cells,
      (ii) transcribing the RNA to cDNA;
      (iii) amplifying from the cDNA a first pool of DNA molecules using a first mixture of oligonucleotides comprising at least two oligonucleotides capable of amplifying HCVR coding regions;
      (iv) amplifying from the cDNA a second pool of DNA molecules using a second mixture of oligonucleotides comprising at least two oligonucleotides capable of amplifying LCVR coding regions; and
      (v) providing pairs of one member of the first pool of DNA molecules and one member of the second pool of DNA molecules;
   or (b):
      (i) isolating RNA from a single B-cell, or from a clonal population of B-cells,
      (ii) transcribing the RNA to cDNA;
      (iii) amplifying from the cDNA a first DNA molecule using a first mixture of oligonucleotides comprising at least two oligonucleotides capable of amplifying HCVR coding regions;
      (iv) amplifying from the cDNA a second DNA molecule using a second mixture of oligonucleotides comprising at least two oligonucleotides capable of amplifying LCVR coding regions;
      (v) generating a first pool of DNA molecules by randomizing at least one codon of the first DNA molecule,
      (vi) generating a second pool of DNA molecules by randomizing at least one codon of the second DNA molecule, and
      (vii) providing pairs of one member of the first pool of DNA molecules and one member of the second pool of DNA molecules;
   or (c):
      (i) isolating RNA from a single B-cell, or from a clonal population of B-cells,
      (ii) transcribing the RNA to cDNA;
      (iii) amplifying from the cDNA a first DNA molecule using a first mixture of oligonucleotides comprising at least two oligonucleotides capable of amplifying HCVR coding regions;
      (iv) amplifying from the cDNA a second DNA molecule using a second mixture of oligonucleotides comprising at least two oligonucleotides capable of amplifying LCVR coding regions;
      (v) generating a pool of DNA molecules by randomizing at least one codon of the first DNA molecule, and
      (vi) providing pairs of one member of the pool of DNA molecules and the second DNA molecule;
   or (d):
      (i) isolating RNA from a single B-cell, or from a clonal population of B-cells,
      (ii) transcribing the RNA to cDNA;
      (iii) amplifying from the cDNA a first DNA molecule using a first mixture of oligonucleotides comprising at least two oligonucleotides capable of amplifying HCVR coding regions;
      (iv) amplifying from the cDNA a second DNA molecule using a second mixture of oligonucleotides comprising at least two oligonucleotides capable of amplifying LCVR coding regions;
      (v) generating a pool of DNA molecules by randomizing at least one codon of the second DNA molecule, and
      (vi) providing pairs of one member of the pool of DNA molecules and the first DNA molecule.
146. The method according to any one of items 125 to 145, characterized in that the variability of antigen-specific antibodies is increased by randomly combining different light and heavy chain variable regions.
147. The eukaryotic cell library according to any one of items 125 to 146, characterized in that the population of isolated B-cells or the single B-cells or the clonal population of B-cells is derived from a source selected from: (a) blood; (b) secondary lymphoid organs, especially spleen or lymph node; (c) bone marrow; and (d) tissue comprising memory B-cells.
148. The eukaryotic cell library according to item 147, characterized in that the population of isolated B-cells comprises or especially consists of peripheral blood mononuclear cells (PBMCs).
149. The eukaryotic cell library according to any one of items 125 to 148, characterized in that the animal is a mammal, especially a rat, a mouse, a rabbit, or a human.
150 The eukaryotic cell library according to item 149, characterized in that the animal is a transgenic mouse or a transgenic rabbit or a human.
151. The eukaryotic cell library according to any one of items 125 to 150, characterized in that the selecting from the population of isolated B-cells a sub-population of B-cells or a single B-cell comprises the steps of:
   (a) contacting the population of isolated B-cells with the antigen of interest or a fragment or antigenic determinant thereof; and
   (b) selecting B-cells or a single B-cell that specifically binds the antigen of interest or fragment or antigenic determinant thereof
152. The eukaryotic cell library according to any one of items 125 to 151, characterized in that the selecting from the population of isolated B-cells a sub population of B-cells or a single B-cell comprises the steps of:
   (a) coating a carrier with the antigen of interest or fragment or antigenic determinant thereof;
   (b) contacting the population of isolated B-cells with the carrier and allowing the B-cells to bind to the carrier via the antigen of interest or fragment or antigenic determinant thereof;
   (c) removing unbound B-cells, wherein especially the carrier comprises or further especially consists of beads, wherein still further especially the beads are paramagnetic beads; and
   (d) recovering the sub-populations of B-cells or the single B-cell from the paramagnetic beads.
153. The eukaryotic cell library according to any one of items 125 to 152, characterized in that the selecting from the population of isolated B-cells a sub population of B-cells or a single B-cell is performed by FACS sorting.
154. The eukaryotic cell library according to any one of items 125 to 153, characterized in that the selecting from the population of isolated B-cells a sub population of B-cells or a single B-cell comprises the steps of:
   (a) contacting the population of isolated B-cells with the antigen of interest or fragment or antigenic determinant thereof, wherein the antigen of interest or fragment or antigenic determinant thereof is labeled with a fluorescence dye; and
   (b) separating B-cells bound to the antigen of interest or fragment or antigenic determinant thereof by FACS sorting.
155. The eukaryotic cell library according to any one of items 125 to 154, characterized in that the selecting from the population of isolated B-cells a sub population of B-cells or a single B-cell comprises the steps of:
   (a) contacting the population of isolated B-cells with the antigen of interest or a fragment or antigenic determinant thereof;
   (b) selecting a population of B-cells or a single B-cell that specifically binds the antigen of interest or fragment or antigenic determinant thereof; and
   (c) selecting the B-cells for at least one additional parameter, wherein especially the selection for the at least one additional parameter is
      (i) a positive selection for a parameter selected from presence of a B cell specific marker, especially CD19 or B220, and vitality of the B-cells; and/or
      (ii) a negative selection for a parameter selected from: presence of IgM antibodies; presence of IgD antibodies, presence of cell death markers, and presence of apoptosis markers.
156. The eukaryotic cell library according to any one of items 125 to 155, characterized in that the selecting from the population of isolated B-cells a sub population of B-cells further comprises the step of selecting for class switched B-cells, especially for IgM- and/or IgD-negative B-cells, most especially for IgM- and IgD-negative B-cells.
157. The eukaryotic cell library according to any one of items 125 to 156, characterized in that the selecting from the population of isolated B-cells a sub population of B-cells or a single B-cell comprises the steps of:
   (a) contacting the population of isolated B-cells with the antigen of interest or fragment or antigenic determinant thereof, wherein the antigen of interest or fragment or antigenic determinant thereof is labeled with a first fluorescence dye, wherein especially the fluorescence dye is Alexa 647 nm, Alexa 488 or Alexa 546 nm;
   (b) contacting the cells of the population of isolated B-cells with anti-IgM and/or anti-IgD antibodies, wherein the anti-IgM and/or anti-IgD antibodies are labeled with a second and/or a third fluorescence dye, wherein the second and/or the third fluorescence dye emits fluorescence at a wavelength which is different from the wavelength of the fluorescence emitted by the first fluorescence dye; and
   (c) separating a population of B-cells or a single B-cell bound to the antigen of interest or fragment or antigenic determinant thereof but not bound to the anti-IgM and/or not bound to the anti-IgD antibodies by FACS sorting.
158. The eukaryotic cell library according to any one of items 125 to 157, characterized in that the library is a viral library, especially an lentiviral library, and the introducing the library into a first population of eukaryotic, especially mammalian cells is performed by infecting the eukaryotic, especially mammalian cell with the viral library, especially with the lentiviral library, wherein further especially the infecting is performed at a multiplicity of infection of at most 10, especially at most 1, more especially at most 0.2, and most especially at most 0.1.
159. The eukaryotic cell library according to item 158, characterized in that the multiplicity of infection is about 0.1.
160. The eukaryotic cell library according to any one of items 125 to 159, characterized in that the isolating of the cell is performed by FACS sorting. In one embodiment the isolating of the cell comprises the steps of:
   (a) staining the first population of eukaryotic, especially mammalian cells with the antigen of interest or fragment or antigenic determinant thereof, wherein the antigen of interest or fragment or antigenic determinant thereof is labeled with a fluorescence dye; and
   (b) separating an individual cell that specifically binds the antigen of interest, or fragment or antigenic determinant thereof, by means of FACS sorting.
161. The eukaryotic cell library according to any one of items 125 to 160, characterized in that the separating an individual cell that specifically binds the antigen of interest, or fragment or antigenic determinant thereof, by means of FACS sorting comprises the step of further selecting the cell at least one additional parameter.
162. The eukaryotic cell library according to item 161, characterized in that the at least one additional parameter is selected from
   (i) a positive selection for vitality of the cell; and/or
   (ii) a negative selection for a parameter selected from: presence of IgM antibodies; presence of IgD antibodies, presence of cell death markers, and presence of apoptosis markers.
163. The eukaryotic cell library according to any one of items 125 to 162, characterized in that the method further comprises the steps of:
   (a) cultivating at least one, especially exactly one, of the individual cells in the presence of a second population of eukaryotic cells, especially mammalian cells;
   (b) verifying the capability of the second population of eukaryotic, especially mammalian cells of that specifically binds the antigen of interest, or fragment or antigenic determinant thereof
164. The eukaryotic cell library according to any one of items 125 to 163, characterized in that the first population of eukaryotic, especially mammalian cells and/or, especially and, the second population of eukaryotic, especially mammalian cells comprises or especially consists of cells selected from: (a) BHK 21 cells, especially ATCC CCL-10; (b) Neuro-2a cells; (c) HEK-293T cells, especially ATCC CRL-11268; (d) CHO-Kl cells, especially ATCC CRL-62; and (e) HEK293 cells.
165. The eukaryotic cell library according to any one of items 125 to 164, characterized in that the first population of eukaryotic, especially mammalian cells and/or the second population of eukaryotic, especially mammalian cells comprises or especially consists of CHO-Kl cells, wherein further especially the expression library is an lentiviral expression library.
166. A workflow/method for the display of full length antibodies comprising a common light chain on the surface of eukaryotic cells and the selection of cells and thereby the selection of an antibody comprising the following steps:
   immunization of an experimental animal, such as a transgenic rabbit,
   selection of antigen-specific B-cells (by FACS, bulk sort),
   PCR amplification of heavy chain encoding nucleic acid: two separate polymerase chain reactions introducing unique restriction sites to enable directed cloning into the shuttle vector, one for the knob chain using one or more or all of the primer of SEQ ID NO: 6 to SEQ ID NO: 10 and the primer of SEQ ID NO: 11 and one for the hole chain using one or more or all of the primer of SEQ ID NO: 1 to SEQ ID NO: 4 and the primer of SEQ ID NO: 5; ligation: first heavy chain variable domain encoding nucleic acid into hole-locus without transmembrane domain, i.e. of the EV71-IRES and second heavy chain variable domain encoding nucleic acid into knob-locus with a transmembrane domain, i.e. downstream of the EV71-IRES,
   virus generation, infection of a mammalian cell stably expressing a common light chain, selection of bispecific antibodies displayed on the surface of the mammalian cell (by off-rate screening), bulk sort of hits (mammalian cell clones) using FACS,
   PCR of the complete first heavy chain encoding nucleic acid and the variable domain encoding nucleic acid (which has no TM domain) of the second heavy chain including the EV71-IRES and cloning into second shuttle vector without transmembrane domain, using e.g. primers of SEQ ID NO: 29 and SEQ ID NO: 30,
   virus generation, infection of a mammalian cell expressing a common light chain, single cell sort of cells, screening of the bispecific antibody in the supernatant, and selection of bispecific antibody.
167. A workflow/method for the display of full length antibodies comprising a common light chain on the surface of eukaryotic cells and the selection of cells and thereby the selection of an antibody comprising the following steps:
   immunization of an experimental animal, such as a transgenic rabbit,
   selection of antigen-specific B-cells (by FACS, bulk sort),
   PCR amplification of heavy chain encoding nucleic acid (two separate polymerase chain reactions introducing unique restriction sites to enable directed cloning into the shuttle vector: ligation: first heavy chain variable domain encoding nucleic acid into hole-locus with transmembrane-domain, i.e. upstream of the EV71-IRES, and second heavy chain variable domain encoding nucleic acid into knob-locus with a transmembrane domain, i.e. downstream of the EV71-IRES,
   virus generation, infection of a mammalian cell expressing a common light chain, selection of mammalian cell membrane-displayed bispecific antibodies (by off-rate screening), bulk sort of hits (mammalian cell clones) using FACS,
   PCR of the complete first heavy chain encoding nucleic acid and the variable domain of the second heavy chain encoding nucleic acid (2.2 kbp) including the EV71-IRES and cloning into second shuttle vector without transmembrane-domain; removal of the transmembrane-domain of the first heavy chain by restriction cutting and religation of the vector,
   virus generation, infection of a mammalian cell expressing a common light chain, single cell sort of cells, screening of the bispecific antibody in the supernatant, selection of bispecific antibody.
168. A workflow/method for the display of full length antibodies comprising a common light chain on the surface of eukaryotic cells and the selection of cells and thereby the selection of an antibody comprising the following steps:
   immunization of an experimental animal, such as a transgenic rabbit,
   selection of antigen-specific B-cells (by FACS, bulk sort),
   PCR amplification of heavy chain encoding nucleic acid: two separate polymerase chain reactions introducing unique restriction sites to enable directed cloning into the shuttle vectors; ligation: first heavy chain variable domain encoding nucleic acid into a first shuttle vector into a hole-locus with or without transmembrane domain, and second heavy chain variable domain encoding nucleic acid into a second shuttle vector into a knob-locus with or without a transmembrane domain, but at least one has a transmembrane domain,
   virus generation (one for the first shuttle vector and one for the second shuttle vector), sequential infection of a mammalian cell expressing a common light chain with the first virus and the second virus, selection of bispecific antibodies displayed on the surface of the mammalian cell (by off-rate screening), bulk sort of hits (mammalian cell clones) using FACS,
   PCR of the heavy chain variable domains encoding nucleic acid and cloning into a third shuttle vector in a bicistronic expression unit without transmembrane domain and EV71-IRES,
   virus generation, infection of a mammalian cell expressing a common light chain, single cell sort of cells, screening of the bispecific antibody in the supernatant, and selection of bispecific antibody.
169. A workflow/method for the display of full length bispecific antibodies comprising a common light chain on the surface of eukaryotic cells and the selection of such eukaryotic cells and thereby also the selection of a bispecific antibody comprising the following steps:
   a first experimental animal, in one embodiment a transgenic mouse or a transgenic rabbit, is immunized with a first antigen of interest, in one embodiment an extracellular receptor domain, wherein the B-cells of the experimental animal express the same light chain,
   a second experimental animal, in one embodiment a transgenic mouse or a transgenic rabbit, is immunized with a second antigen of interest, in one embodiment an extracellular receptor domain, wherein the B-cells of the experimental animal express the same light chain, whereby the first antigen and the second antigen are different,
   selecting the B-cells of the first and second immunized experimental animal, in one embodiment by bulk sorting by FACS,
   obtaining the heavy chain encoding nucleic acid of each B-cell by individual PCR amplification by two separate/sequential polymerase chain reactions introducing unique restriction sites to enable directed cloning into a shuttle vector/lentiviral expression vector,
   ligation of a first heavy chain variable domain encoding nucleic acid into a shuttle vector/lentiviral expression vector upstream of an IRES, in one embodiment the IRES is the EV71-IRES, into a hole- or knob-locus with transmembrane domain, and ligation of a second heavy chain variable domain encoding nucleic acid into a the same shuttle vector/lentiviral expression vector downstream of the IRES into the respective other locus with a transmembrane domain, i.e. if the heavy chain upstream of the IRES has a hole-locus the heavy chain downstream of the IRES has a knob-locus and vice versa, whereby the first heavy chain variable domain binds to a first antigen and the second variable domain binds to a second antigen, whereby the first antigen and the second antigen are different,
   virus generation,
   infection of a mammalian cell expressing a common light chain with the virus,
   selection of cells displaying bispecific antibodies on their surface by FACS of doubly labeled transduced cells,
   PCR of the complete first heavy chain encoding nucleic acid and the variable domain of the second heavy chain encoding nucleic acid (2.2 kbp) including the EV71-IRES and cloning into second shuttle vector without transmembrane-domain; removal of the transmembrane-domain of the first heavy chain by restriction cutting and religation of the vector,
   virus generation, infection of a mammalian cell expressing a common light chain, single cell sort of cells, screening of the bispecific antibody in the supernatant, and selection of a bispecific antibody.
170. A workflow/method for the display of full length bispecific antibodies comprising a common light chain on the surface of eukaryotic cells and the selection of such eukaryotic cells and thereby also the selection of a bispecific antibody comprising the following steps:
   an experimental animal, in one embodiment a transgenic mouse or a transgenic rabbit, is immunized with an antigen of interest, in one embodiment an extracellular receptor domain, wherein the B-cells of the experimental animal express the same light chain,
   selecting the B-cells of the immunized experimental animal, in one embodiment by bulk sorting by FACS,
   obtaining the heavy chain encoding nucleic acid of each B-cell by individual PCR amplification by two separate/sequential polymerase chain reactions introducing unique restriction sites to enable directed cloning into a shuttle vector/lentiviral expression vector,
   ligation of the heavy chain variable domain encoding nucleic acid into a shuttle vector/lentiviral expression vector downstream of an IRES, in one embodiment the IRES is the EV71-IRES, into a heavy chain locus with transmembrane domain, wherein the shuttle vector/lentiviral expression vector comprises upstream of the IRES the common light chain encoding nucleic acid,
   virus generation,
   infection of a mammalian cell expressing a common light chain with the virus,
   selection of cells displaying antibodies on their surface by FACS of antigen specific labeled transduced cells, in one embodiment by bulk sorting by FACS,
   obtaining the heavy chain encoding nucleic acid of each selected cell by individual PCR amplification by two separate/sequential polymerase chain reactions introducing unique restriction sites to enable directed cloning into a shuttle vector/lentiviral expression vector,
   ligation of a first heavy chain variable domain encoding nucleic acid into a shuttle vector/lentiviral expression vector upstream of an IRES, in one embodiment the IRES is the EV71-IRES, into a hole- or knob-locus without transmembrane domain, and ligation of a second heavy chain variable domain encoding nucleic acid into a the same shuttle vector/lentiviral expression vector downstream of the IRES into the respective other locus without a transmembrane domain, i.e. if the heavy chain upstream of the IRES has a hole-locus the heavy chain downstream of the IRES has a knob-locus and vice versa, whereby the first heavy chain variable domain binds to a first antigen and the second variable domain binds to a second antigen, whereby the first antigen and the second antigen can be the same or different,
   virus generation,
   infection of a mammalian cell expressing a common light chain with the virus,
   selection of a cell secreting a bispecific antibody.
171. Use of a cell selected with a method according to any one of items 1 to 170 for the production of an antibody.

The following examples, sequences and figures are provided to aid the understanding of the present invention, the true scope of which is set forth in the appended claims. It is understood that modifications can be made in the procedures set forth without departing from the spirit of the invention.

### Sequences

SEQ ID NO: 01 to 30 PCR primer
SEQ ID NO: 31 EV71-IRES
SEQ ID NO: 32 bGH polyA signal sequence
SEQ ID NO: 33 human CMV promoter
SEQ ID NO: 34 intron 6+M1/M2 sequence
SEQ ID NO: 35 M1/M2 sequence
SEQ ID NO: 36 to 39 PCR primer.

### FIGURES

FIG. 1 FACS-dotplots of IRES-linked expression of GFP; a) gtx-IRES, b) EV71-IRES, c) ELF4G-IRES, d) EMCV-IRES.

FIG. 2 Comparison of IRES-linked expression of antibody LC and HC; a) gtx-IRES, b) EV71-IRES, c) ELF4G-IRES, d) EMCV-IRES; lower figure: scheme of the expression construct.

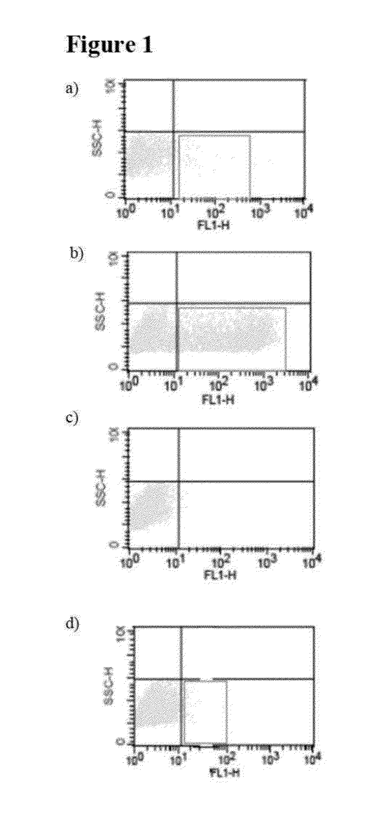

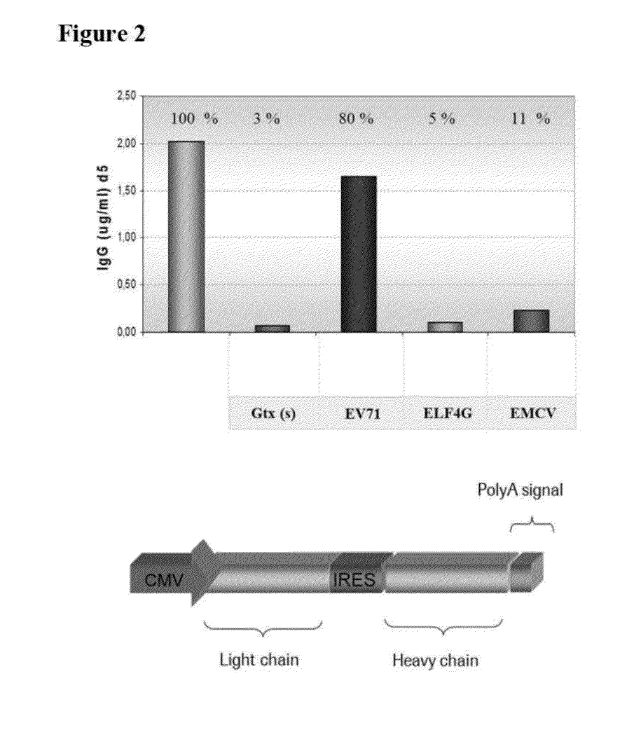

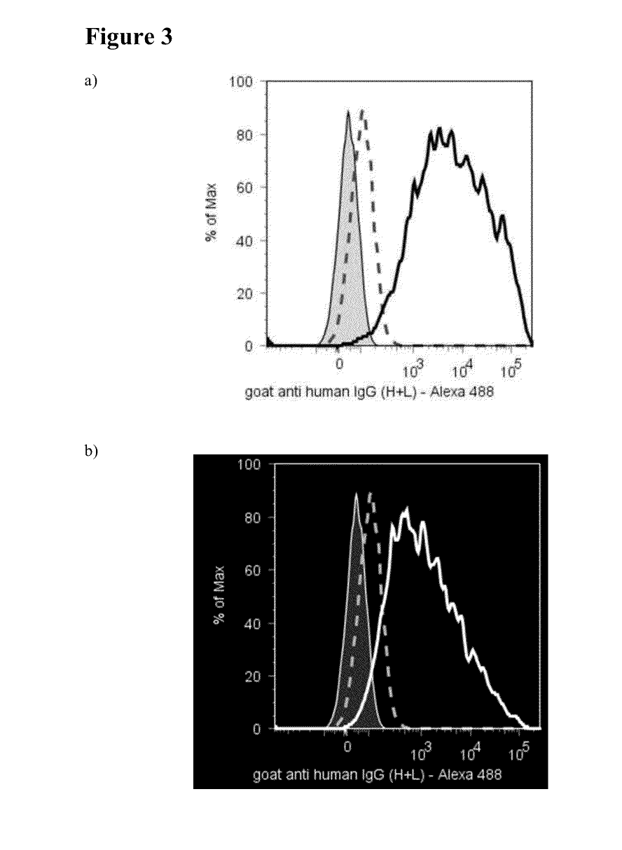

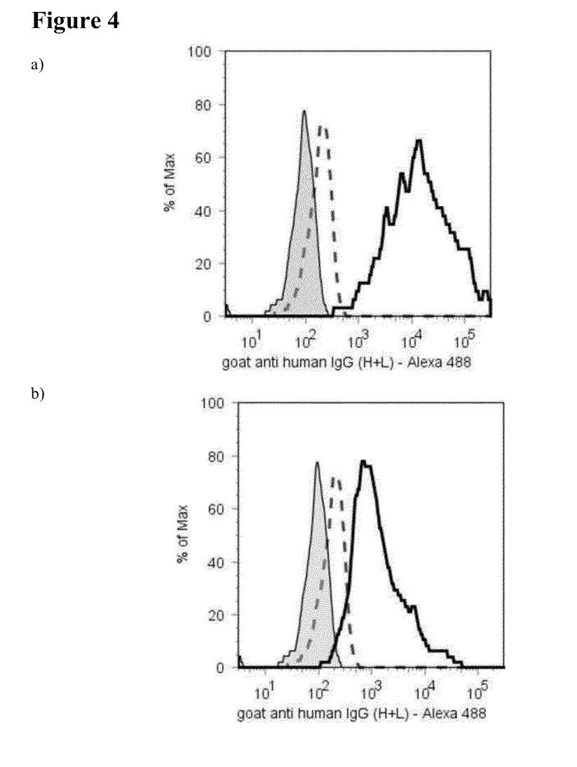

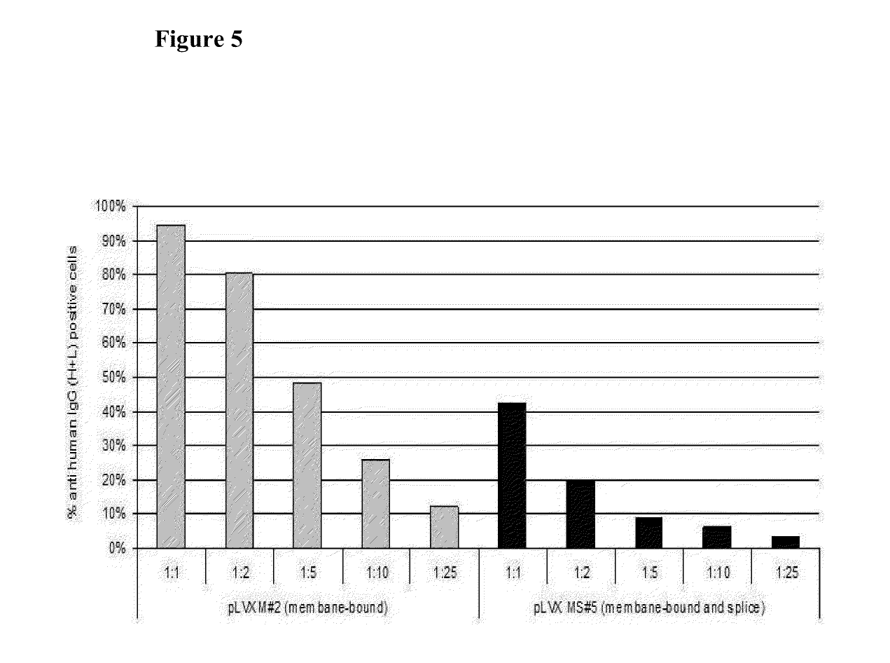

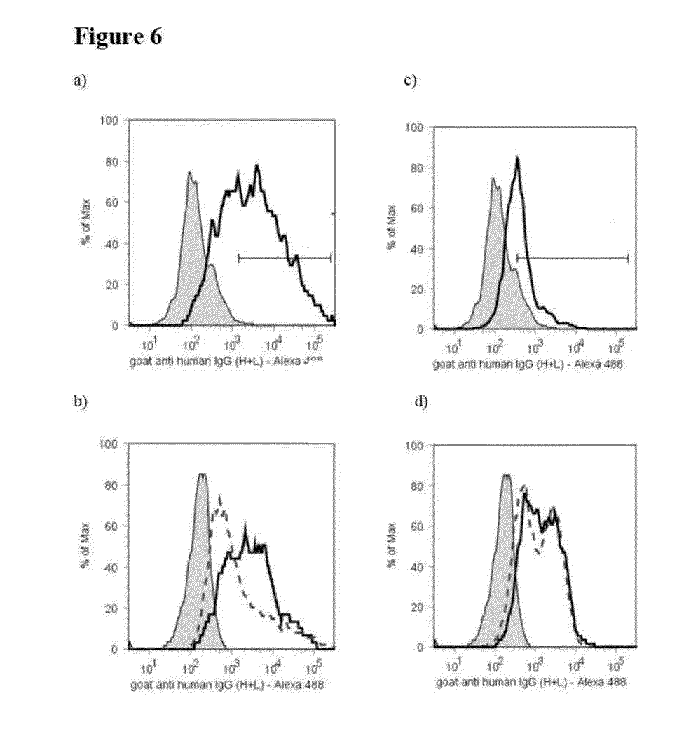

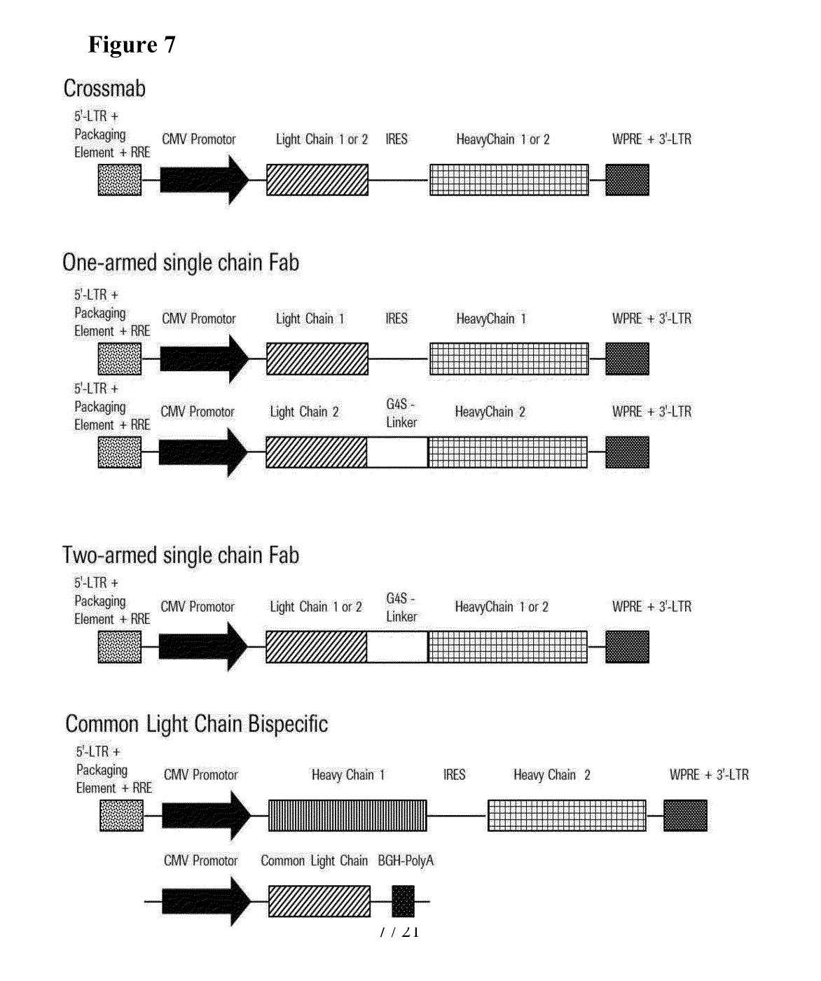

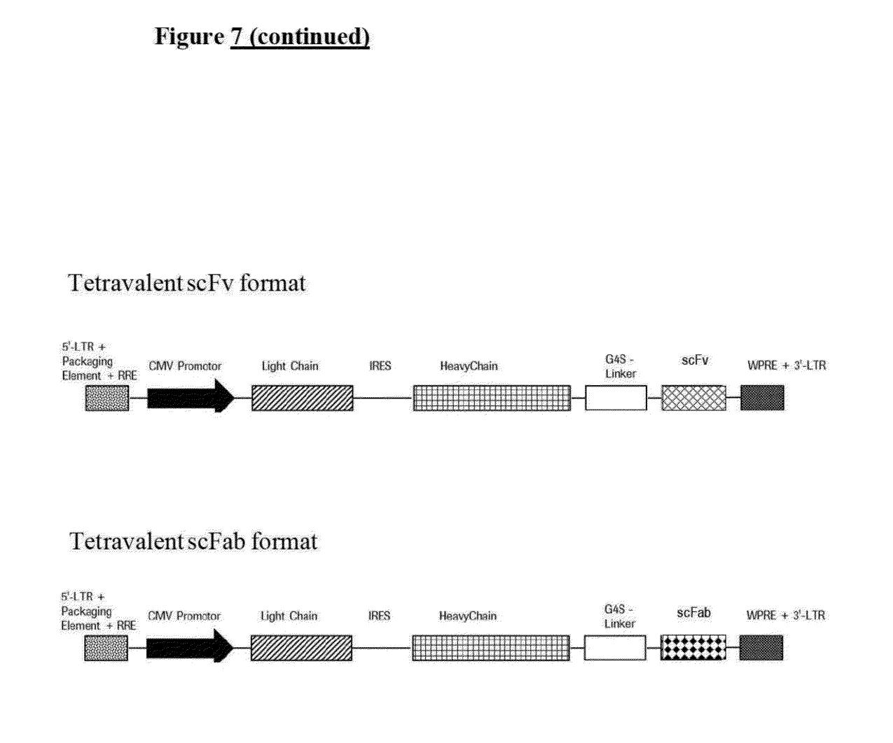

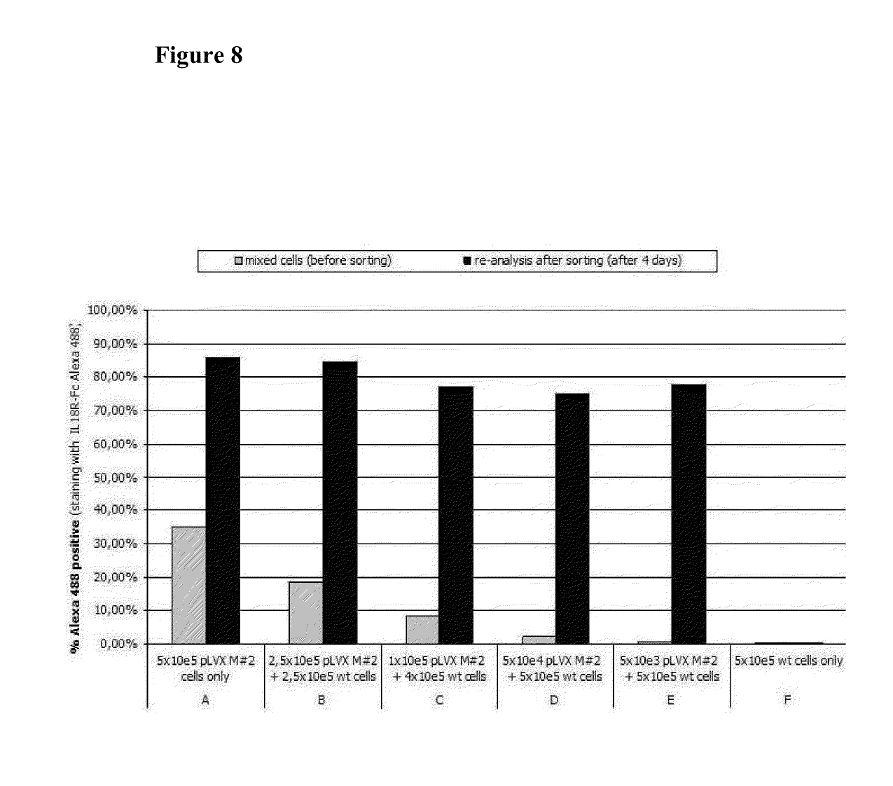

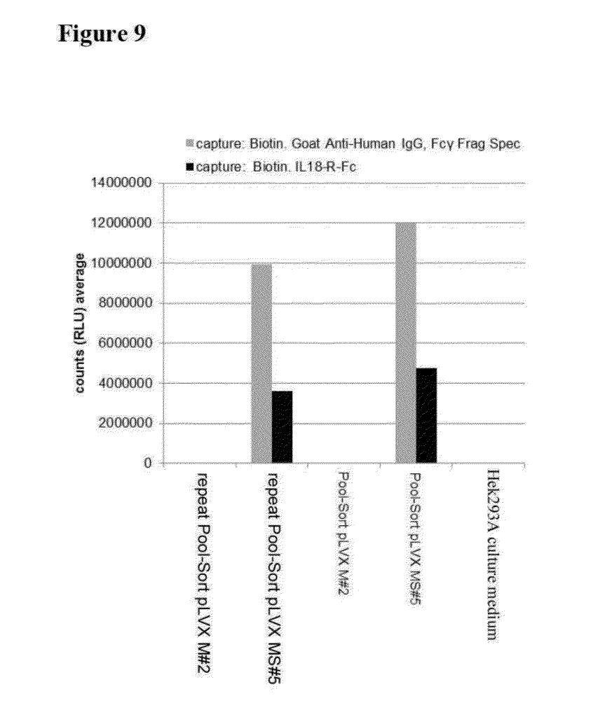

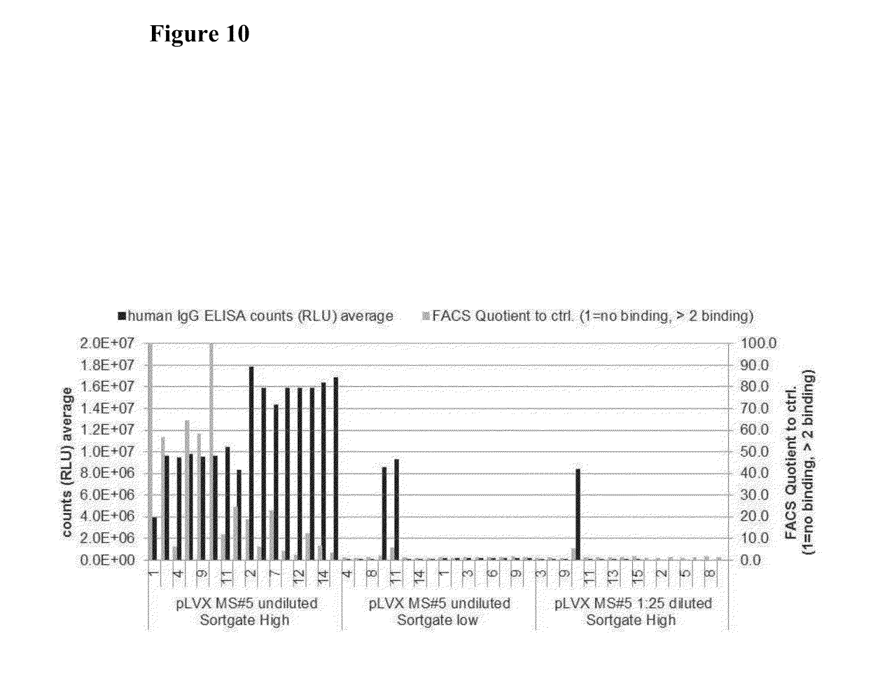

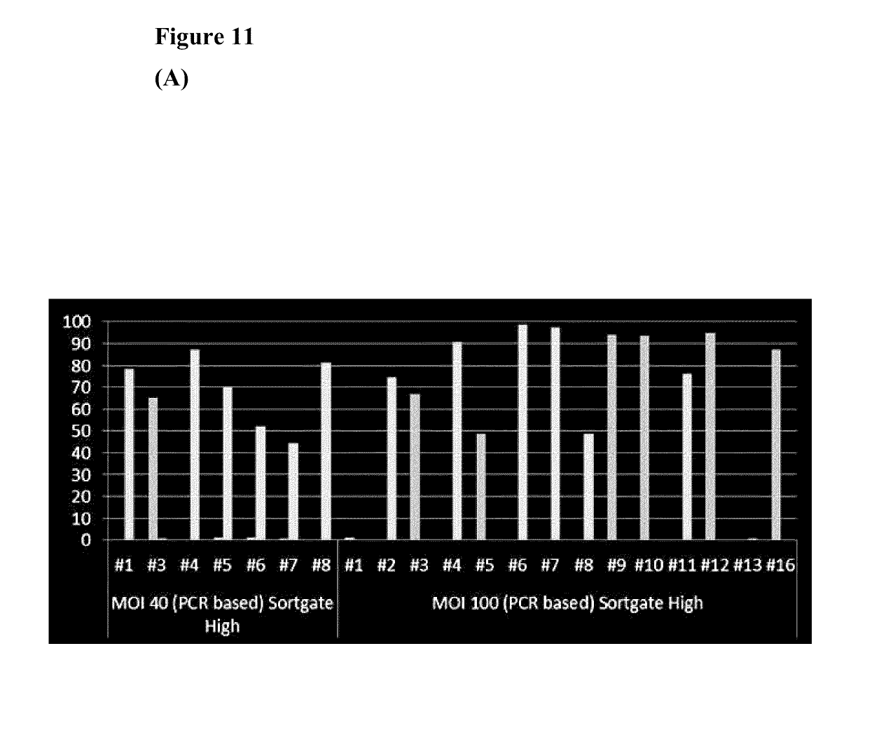

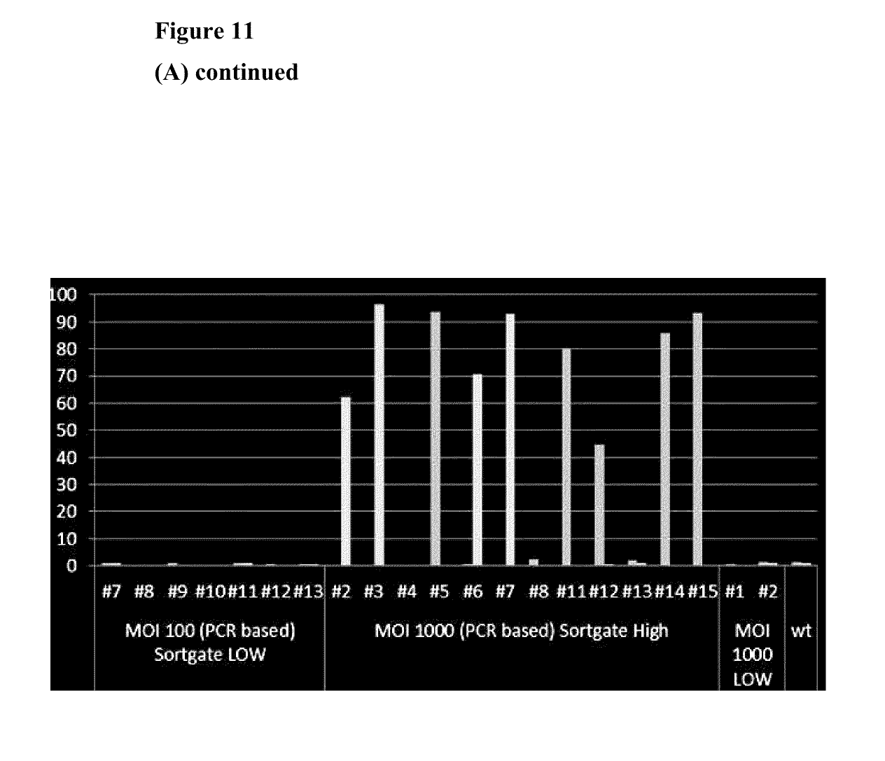

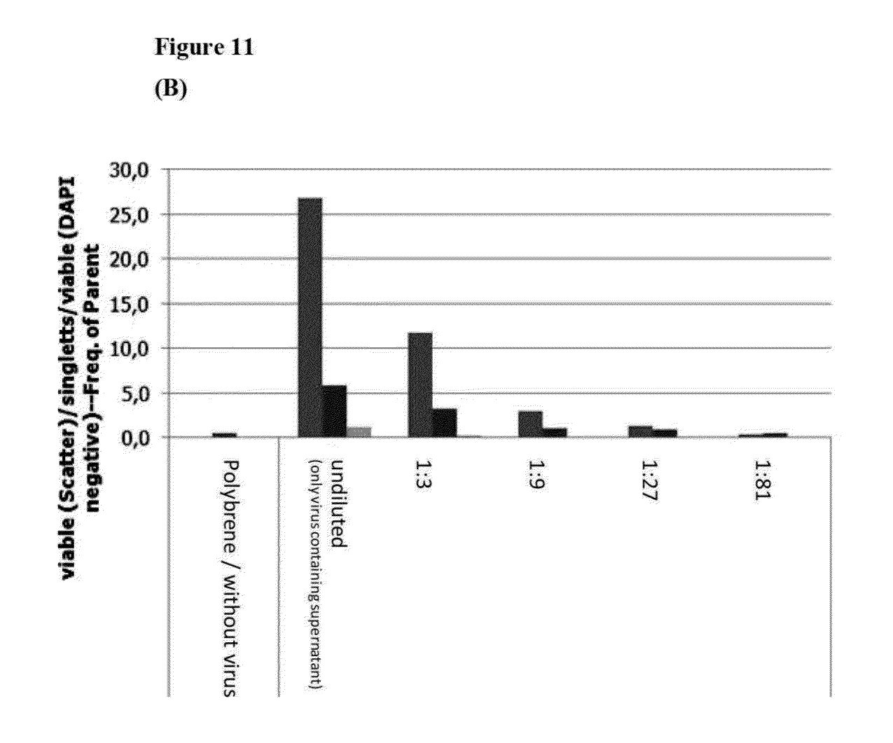

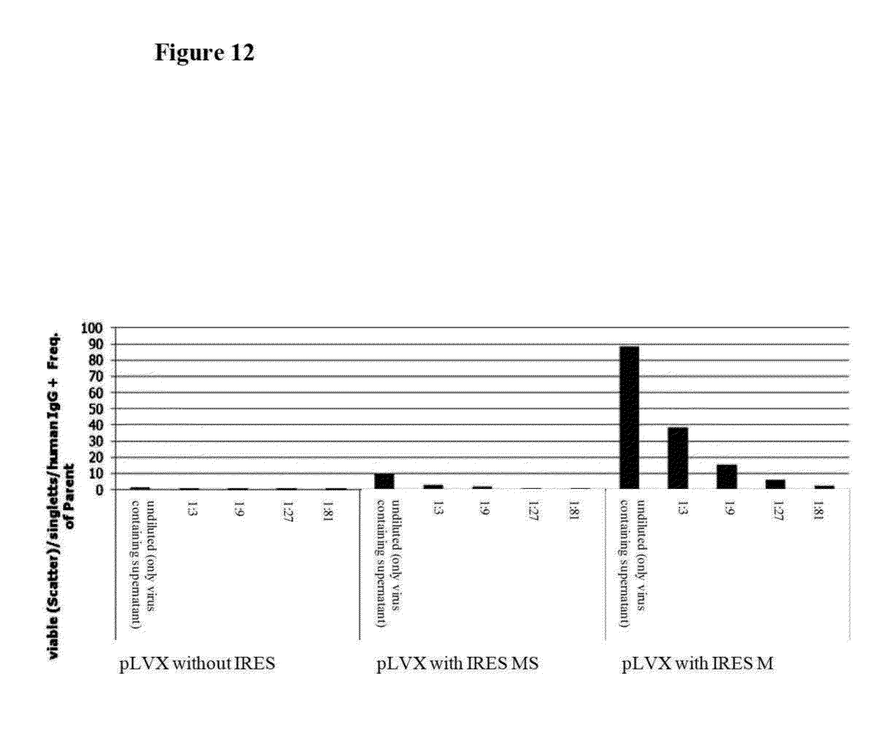

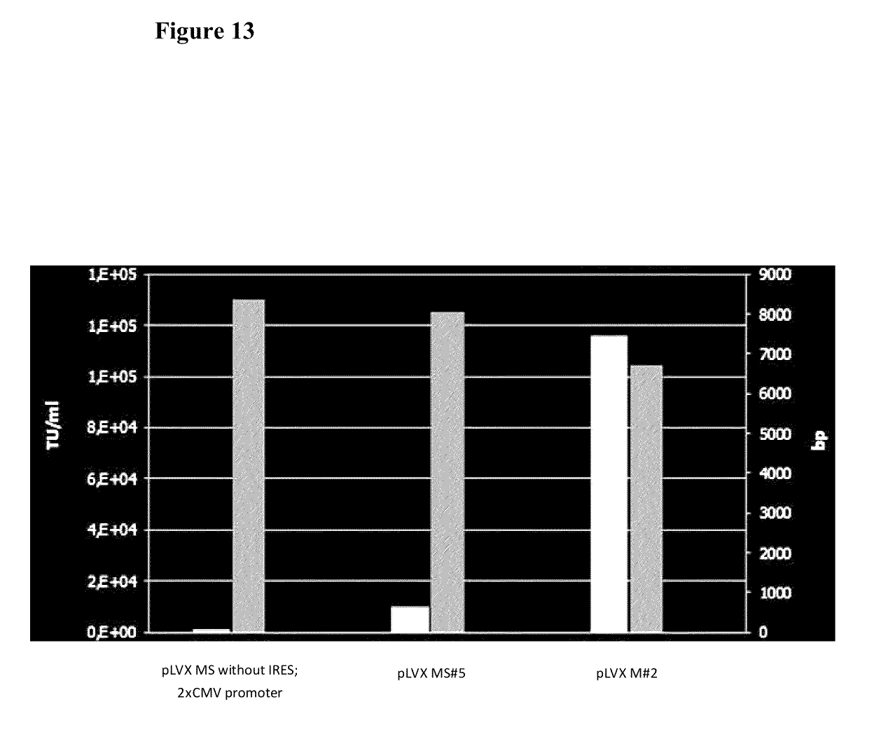

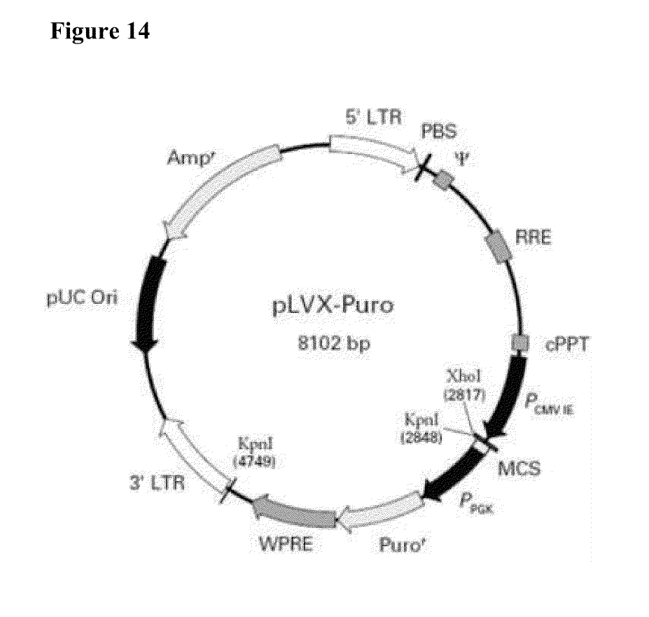

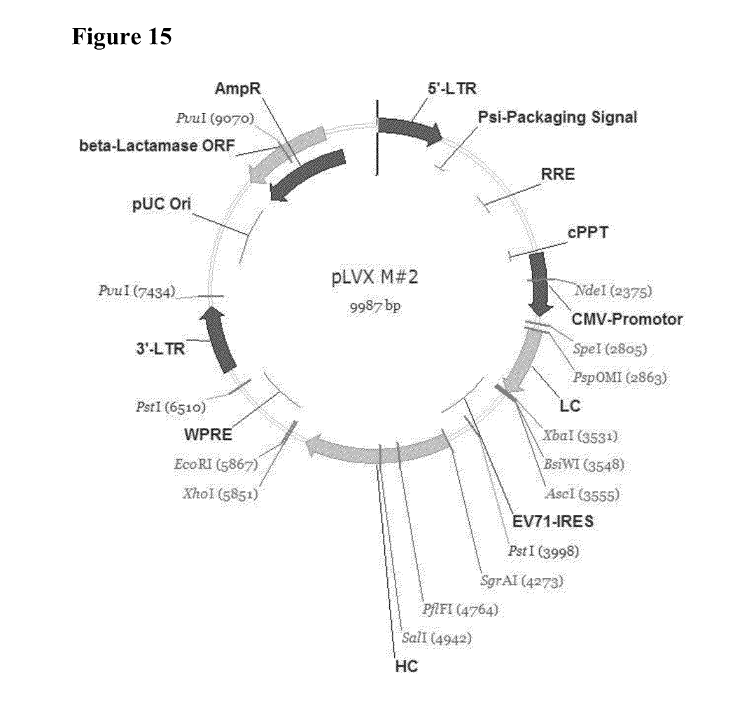

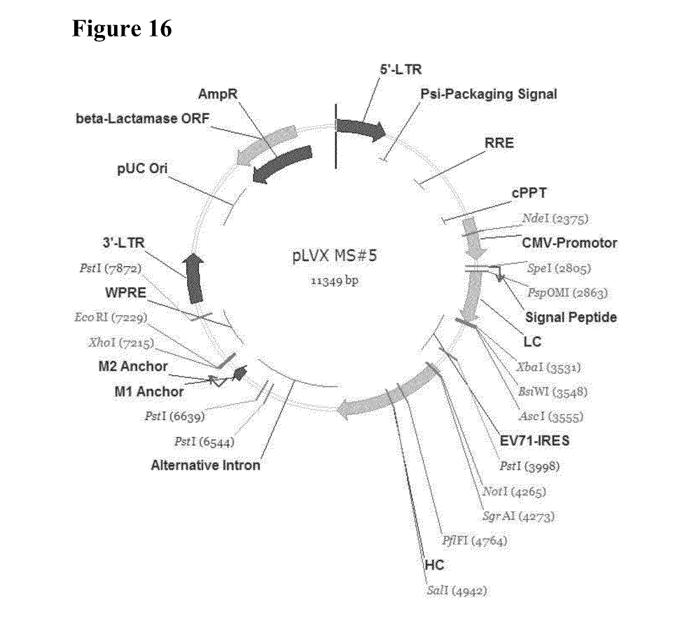

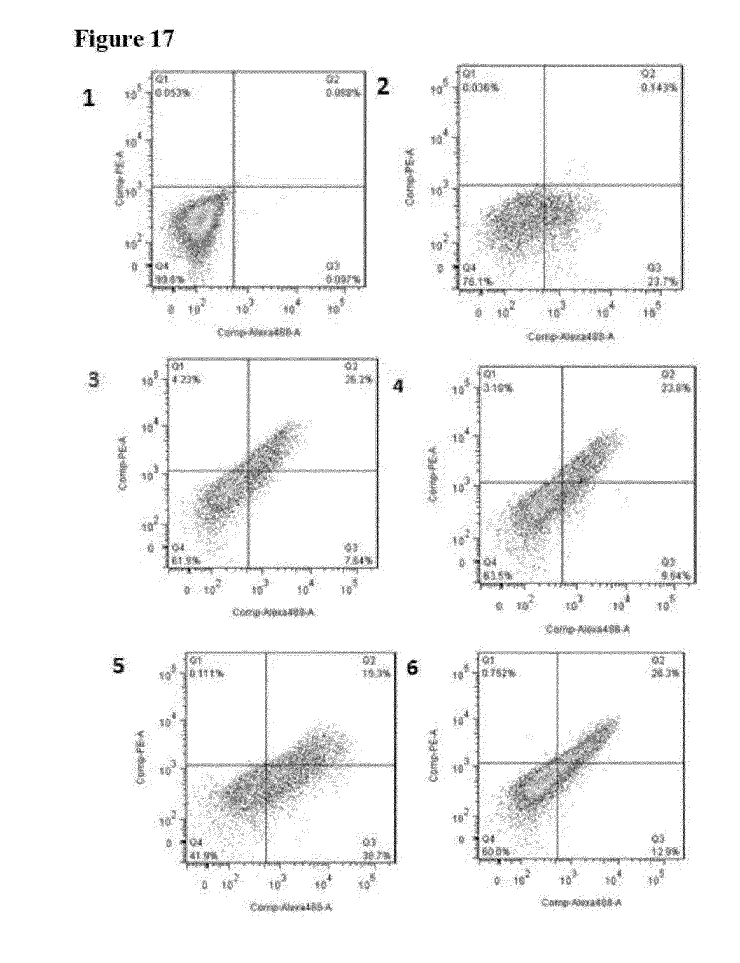

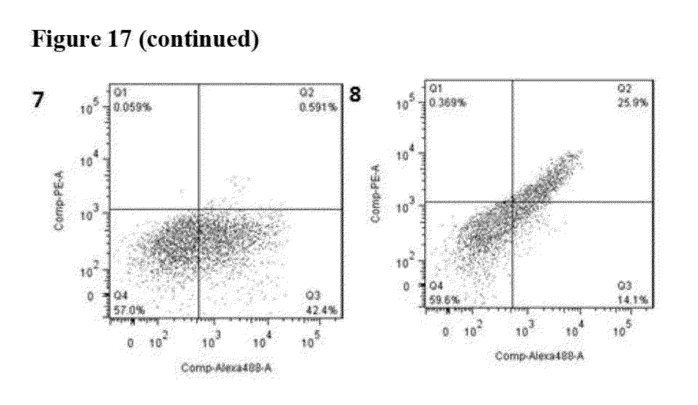

FIG. 3 FACS histograms of transiently transfected HEK293 cells obtained 24 hours after transfection;
a) transient transfection with pLVX M#2 (membrane-bound IgG): grey filled histogram: autofluorescence fectin control, dotted line histogram: anti-human IgG (H+L) antibody-Alexa 488 stained cells, solid line histogram: anti-human IgG (H+L) antibody-Alexa 488 stained cells after transfection with pLVX M#2;
b) transient transfection with pLVX MS#5 (membrane-bound and secreted): grey filled histogram: autofluorescence fectin control, dotted line histogram: anti-human IgG (H+L) antibody-Alexa 488 stained cells, solid line histogram: anti-human IgG (H+L) antibody-Alexa 488 stained cells after transfection with pLVX MS#5.

FIG. 4 FACS histograms of virally transduced HEK293 cells obtained 96 hours after transduction;
a) viral transduction with pLVX M#2 virus: grey filled histogram: autofluorescence polybrene control, dotted line histogram: anti-human IgG (H+L) antibody-Alexa 488 stained cells, solid line histogram: anti-human IgG (H+L) antibody-Alexa 488 stained cells after transduction with pLVX M#2 virus;
b) viral transduction with pLVX MS#5 virus: grey filled histogram: autofluorescence fectin control, dotted line histogram: anti-human IgG (H+L) antibody-Alexa 488 stained cells, solid line histogram: anti-human IgG (H+L) antibody-Alexa 488 stained cells after transduction with pLVX MS#5 virus.

FIG. 5 Determination of lentiviral titer by tittering lentiviral stock solutions using flow cytometry analysis at 96 hours post transduction with freshly harvested lentiviral supernatants.

FIG. 6 FACS histograms of virally transduced HEK293 cells directly after transduction and 14 or 28 days, respectively later;
a) sorting (black bar area) of anti-human IgG (H+L) antibody-Alexa 488 conjugate positive HEK293 cells 96 hours after viral transduction with pLVX M#2 virus: grey filled histogram: polybrene control; solid line histogram: transduced cell line;
b) re-analysis of sorted cells by FACS staining 14 and 28 days after initial sort after transduction with pLVX M#2 virus: grey filled histogram: polybrene control; dotted line histogram: transduced cell line analyzed for the second time 14 days after the first sort; solid line histogram: transduced cell line analyzed for the second time 28 days after the first sort;
c) sorting (back bar) of anti-human IgG (H+L) antibody-Alexa 488 conjugate positive HEK293 cells 96 hours after viral infection with pLVX MS#5: grey filled histogram: polybrene control; solid line histogram: transduced cell line;
d) re-analysis of sorted cells by FACS staining 14 and 28 days after initial sort after transduction with pLVX MS#5 virus: grey filled histogram: polybrene control; dotted line histogram: transduced cell line analyzed for the second time 14 days after the first sort; solid line histogram: transduced cell line analyzed for the second time 28 days after the first sort.

FIG. 7 Bispecific antibody expression cassettes.

FIG. 8 Recovery of HEK293A cell presenting membrane bound antibody by FACS sorting of cells labeled with Alexa-488 antigen conjugate.

FIG. 9 ELISA results of supernatants from pLVX M#2 or MS#5 positive sorted cells (pool-sort).

FIG. 10 Results of comparative analysis by FACS, ELISA of single deposited FACS positive cells.

FIG. 11 Results of the staining of cells infected in the presence of two viruses harboring plasmids for the membrane-bound expression of IgGs directed against two different antigens:
(A) left bar—single cell level: antigen 1 positive cells; right bar antigen 2 positive cells; no double infection detectable for MOI 100 with high sort gate; y-axis: viable (scatter)/singlets-freq. of parent;
(B) left bar—pool level: antigen 1 positive cells; middle bar: antigen 2 positive cells; right bar: antigen 1 and antigen 2 positive cells.

FIG. 12 FACS analysis of cells transduced with different lentiviral particles: left—pLVX MS without IRES, two hCMV promoter containing separated expression cassettes for expression of membrane-bound and secreted full length antibody; middle—pLVX MS with IRES, one bicistronic expression cassette with one hCMV promoter for expression of membrane-bound and secreted full length antibody; right—pLVX M with IRES, one bicistronic expression cassette with one hCMV promoter for expression of membrane-bound full length antibody.

FIG. 13 FACS analysis of TU/ml depending on the size (bp) of the lentiviral expression vector; left bar—TU/ml; right bar—size in by of lentiviral expression vector.

FIG. 14 Vector map pLVX-puro.

FIG. 15 Vector map pLVX M#2.

FIG. 16 Vector map pLVX MS#5.

FIG. 17 FACS analysis of HEK293 cells transfected with bispecific display vectors encoding antibodies with no or one transmembrane domain; V1.1: M-B(knob)-IRES-M-B(hole) (membrane anchor on both binder heavy chains), V1.2: M-B(knob)-IRES-M-N(hole) (membrane anchor on both heavy chains, binder and non-binder), V1.3: M-N(knob)-IRES-B(hole) (membrane anchor on non-binder only), V1.4: B(knob)-IRES-M-N(hole) (membrane anchor on non-binder only), V1.5: M-N(knob)-IRES-M-N(hole) (non-binder only, membrane anchor on both heavy chains), V1.6 B(hole)-IRES-M-N(knob) (membrane anchor on non-binder, knob and hole exchanged); 1: control 1—fectin only, 2: control 2—common LC only, 3: V1.1 M-B-IRES-M-B+common LC, 4: V1.2 M-B-IRES-M-N+common LC, 5: V1.3 M-N-IRES-B+common LC, 6: V1.4 B-IRES-M-N+common LC, 7: V1.5 M-N-IRES-M-N+common LC, 8: V1.6 B-(hole)-IRES-M-N(knob)+common LC.

### EXAMPLE 1

### Construction of Vectors pLVX M#2 and pLVX MS#5

Shuttle Vector: Removal of the MCS, the P_{PGK} promoter and the puromycin resistance gene. Insertion of the light chain—EV71-IRES—heavy chain—alternative splice element with the transmembrane domain into the plasmid.

The starting shuttle vector comprises the following elements:

| | element | source |
|---|---|---|
| 1 | 5′ LTR | Human Immunodeficiency Virus-1 |
| 2 | PBS (primer binding site) | Simian Virus 40 |
| 3 | psi (ψ) | Human Immunodeficiency Virus-1 |
| 4 | RRE | Human Immunodeficiency Virus-1 |
| 5 | cPPT | Human Immunodeficiency Virus |
| 6 | PCMV IE | Human Cytomegalovirus |
| 7 | MCS | synthetic |
| 8 | PPGK | Saccharomyces cerevisiae |
| 9 | Puromycin | Streptomyces alboniger |
| 10 | WPRE | Hepatitis Virus |
| 11 | 3′ LTR | Human Immunodeficiency Virus-1 |
| 12 | pUC ori | E. coli |
| 13 | Ampicillin | E. coli |

Plasmid pLVX M#2 comprises the following elements:

| | element | source |
|---|---|---|
| 1 | 5′ LTR | Human Immunodeficiency Virus-1 |
| 2 | PBS (primer binding site) | Simian Virus 40 |
| 3 | psi (ψ) | Human Immunodeficiency Virus-1 |
| 4 | RRE | Human Immunodeficiency Virus-1 |
| 5 | cPPT | Human Immunodeficiency Virus |
| 6 | PCMV IE | Human Cytomegalovirus |
| 7 | IgG Light Chain | Human |
| 8 | EV71-IRES | EV71 Virus |
| 9 | IgG Heavy Chain | Human |
| 10 | WPRE | Hepatitis Virus |
| 11 | 3′ LTR | Human Immunodeficiency Virus-1 |
| 12 | pUC ori | E. coli |
| 13 | Ampicillin | E. coli |

The plasmid pLVX MS#5 comprises the following elements:

| | element | source |
|---|---|---|
| 1 | 5′ LTR | Human Immunodeficiency Virus-1 |
| 2 | PBS (primer binding site) | Simian Virus 40 |
| 3 | psi (ψ) | Human Immunodeficiency Virus-1 |
| 4 | RRE | Human Immunodeficiency Virus-1 |
| 5 | cPPT | Human Immunodeficiency Virus |
| 6 | PCMV IE | Human Cytomegalovirus |
| 7 | IgG Light Chain | Human |
| 8 | EV71-IRES | EV71 Virus |
| 9 | IgG Heavy Chain with | Human |
| | alternatively splice | |
| | transmembrane domain | |
| | (M1/2) | |
| 10 | WPRE | Hepatitis Virus |
| 11 | 3′ LTR | Human Immunodeficiency Virus-1 |
| 12 | pUC ori | E. coli |
| 13 | Ampicillin | E. coli |

### EXAMPLE 2

### Generation of Infective Viruses

3.75*10⁵ Lenti-X™ 293 T cells were seeded in each well of a 6-well plate and incubated overnight. The next day the cells were co-transfected with 2.5 μg pLVX M#2 or pLVX MS#5 and 12.75 μl Lenti-X HTX Packaging Mix (Clontech 631248) using 20 μl Lipofectamine™ 2000 Transfection Reagent (Invitrogen cat. no.: P/N 52887) for each well. The medium was changed after 24 hours of incubation. The virus containing supernatants were harvested 48 hours post transfection.

### EXAMPLE 3

### Transient Transfection of HEK293 Cells

1*10⁵ Lenti-X™ 293 T cells were seeded in 24-well and incubated overnight. The next day the cells were transfected with 0.9 μg pLVX M#2 or pLVX MS#5 and 2.7 μl Lipofectamine™ 2000 Transfection Reagent (Invitrogen cat. no.: P/N 52887) in each well. Staining was performed with goat anti human IgG (H+L)—Alexa 488 conjugate (Invitrogen cat. no. A11013) and FACS analysis was performed 24 hours post transfection.

Results are shown in FIG. 3.

### EXAMPLE 4

### Viral Transduction of HEK293 Cells

### Viral Infection/Transduction

1.5*10⁴ HEK293A cells seeded in the wells of a 48-well plate were incubated overnight. The next day the complete medium was removed and the cells were infected with 300 μl undiluted virus containing supernatants in presence of 8 μg/m polybrene. The medium was changed after 24 hours of incubation. The staining of the cells was done with goat anti-human IgG (H+L) antibody-Alexa 488 conjugate (Invitrogen cat. no. A11013) and FACS analysis was performed 96 hours post transfection.

Results are shown in FIG. 4.

### Determination of Lentiviral Titer

Lentiviral titers were determined by measuring lentiviral stock solutions using flow cytometry analysis at 96 hours post transduction with freshly harvested lentiviral supernatants.

| | Lentivirus Dilution | % positive cells | TU/ml |
|---|---|---|---|
| pLVX M #2 | 1:1 | 95% | 6.3E+04 |
| (membane- | 1:2 | 81% | 1.2E+05 |
| bound) | 1:5 | 49% | 1.6E+05 |
| | 1:10 | 26% | 1.9E+05 |
| | 1:25 | 12% | 2.0E+05 |
| pLVX MS #5 | 1:1 | 42% | 2.8E+04 |
| (membane- | 1:2 | 20% | 2.9E+04 |
| bound and | 1:5 | 9% | 2.9E+04 |
| splice) | 1:10 | 6% | 4.5E+04 |
| | 1:25 | 4% | 6.1E+04 |

The titer was calculated with the following formula:

TU/ml=F*c*DN

with
F=frequency of positive cells
C=total number of cells in well at time of transduction (e.g. 200,000 cells)
V=volume of inoculum in ml (0.3 ml)
D=lentivirus Dilution
TU=transducing units

Results are shown in FIG. 5.

### EXAMPLE 5

### STABILITY OF VIRAL TRANSDUCED HEK293 CELL LINE

Virus generation and viral infection/transduction was performed as described in the previous Examples 2 and 4.

Sorted cells were expanded in 6-well plates or T75 shaker flasks. The splitting of cells was performed at 80% confluence. For FACS staining 1×10⁵ cells were incubated with 10 μg/ml goat anti-human IgG (H+L) antibody-Alexa 488 conjugate in 100 μl total volume.

The determination of the long term stability was performed without selection pressure. The corresponding FACS histograms are shown in FIG. 6.

### EXAMPLE 6

### Sorting of a Mixture of Cells Displaying Membrane-Bound Antibody and Wild-Type Cells

HEK293A wild-type cells were mixed with HEK293A cells stably transduced with vector pLVX M#2 (day 28), stained with 250 μl 10 μg/ml Alexa 488 coupled antigen, FACS analysis and sort of Alexa 488 positive cells in 24-well microtiter plates.

Four days after sort, all cells in the wells of the 24-well microtiter plate, i.e. approx. 1*10⁵ cells, were stained with 50 μl 10 μg/ml Alexa 488 coupled antigen followed by FACS analysis.

The results are shown in FIG. 8.

### EXAMPLE 7

### IgG Secretion of Sorted Cells (Membrane-Bound IgG Positive) into Cultivation Supernatant

Sorted cells were expanded in 6-well microtiter plates or T75 flasks. The splitting of cells was carried out at 80% confluence. Supernatant from cells with 95% confluence was used for IgG ELISA. The results are presented in the following Table and in FIG. 9.

TABLE
ELISA results of supernatants from pLVX M #2 or
MS #5 positive sorted cells (pool-sort)

| | capture: Biotin. Goat Anti- | | |
|---|---|---|---|
| | Human IgG, Fcy Frag Spec | | capture: Biotin. |
| | counts | Conc. | IL18-R-Fc |
| | Average | [ng/ml] | counts Average |
| WDHL Pool-Sort | 4.79E+04 | <MIN | 3.0E+04 |
| pLVX M # 2 | | | |
| WDHL Pool-Sort | 9.96E+06 | 317.4 | 3.6E+06 |
| pLVX MS # 5 | | | |
| Pool-Sort | 3.79E+04 | <MIN | 1.9E+04 |
| pLVX M # 2 | | | |
| Pool-Sort | 1.20E+07 | 557.3 | 4.8E+06 |
| pLVX MS # 5 | | | |
| Hek293A | 2.06E+04 | <MIN | 7.4E+03 |
| culture medium | | | |

### EXAMPLE 8

### Correlation of Membrane-Bound Antibody and Secreted Antibody

### Virus Generation

3.75*10⁵ Lenti-X™ 293 T cells were seeded in each well of a 6-well plate and incubated overnight. The next day the cells were co-transfected with 2.5 μg pLVX MS#5 and 12.75 μl Lenti-X HTX Packaging Mix (Clontech 631248) using 20 μl Lipofectamine™ 2000 Transfection Reagent (Invitrogen cat. no.: P/N 52887) for each well. The medium was changed after 24 hours of incubation. The virus containing supernatants were harvested 48 hours post transfection.

### Viral Infection/Transduction

3*10⁴ Hek293A cells seeded in the wells of a 24-well plate were incubated overnight. The next day the complete medium was removed and the cells were infected with 600 μl undiluted or 1:25 diluted virus containing supernatants in presence of 8 μg/ml polybrene. The medium was changed after 24 hours of incubation. The staining of the cells was done with goat anti-human IgG (H+L) antibody-Alexa 488 conjugate (Invitrogen cat. no.: A11013) and FACS analysis was performed 96 hours post transduction and individual cells were sorted into the wells of a 96-well plate.

Three populations of cells were single sorted:
pLVX MS#5 undiluted virus infected cells with high sort gate
pLVX MS#5 undiluted virus infected cells with low sort gate
pLVX MS#5 1:25 diluted virus infected cells with low sort gate.

Sorted cells were grown at 95% confluence from 96-well plates and expanded into 24 well plates. For FACS staining 5*10⁴ cells were incubated with 10 μg/ml goat anti-human IgG (H+L) antibody-Alexa 488 conjugate in 100 μl total volume. From the sorted cells RNA was isolated for PCR analysis for LC (Touchdown PCR).

In FIG. 10 the results are shown.

It can be seen from the compared analysis that FACS analysis of single cell clones stained with anti-human IgG (H+L) antibody Alexa 488 and human IgG ELISA of cultivation supernatants from one single cell clone of each sort gate can both be used for the selection of cells, whereby for FACS a quotient of more than 2 to a control sample is used as threshold (results also shown in the following Table).

TABLE

| | | human IgG | human IgG | FACS Quotient to ctrl. | Touchdown |
|---|---|---|---|---|---|
| | | ELISA counts | concentration | (1 = no binding, >2 | PCR for Light |
| | # of clone | (RLU) average | (ng/ml) | binding) | Chain (Lc) |
| pLVX MS #5 | 1 | 3979272 | >62.5 | 1124.2 | ++++ |
| undiluted | 2 | 17898500 | 43.3 | 18.7 | ++++ |
| Sortgate High | 3 | 9632206 | >62.5 | 56.7 | ++++ |
| | 4 | 9461668 | >62.5 | 6.2 | ++++ |
| | 9 | 9566852 | >62.5 | 58.5 | ++++ |
| pLVX MS #5 | 1 | 218076 | <MIN | 1.2 | − |
| undiluted | 2 | 180654 | <MIN | 1.0 | − |
| Sortgate Low | 3 | 179118 | <MIN | 1.4 | − |
| | 4 | 39302 | 3.2 | 1.2 | + |
| | 5 | 164588 | <MIN | 1.3 | − |
| | 6 | 174238 | <MIN | 1.4 | − |
| | 7 | 50222 | 3.5 | 1.0 | − |
| | 8 | 177218 | <MIN | 1.3 | − |
| | 9 | 173048 | <MIN | 1.6 | − |
| | 10 | 8597740 | >62.5 | 2.0 | ++++ |
| | 11 | 9288676 | >62.5 | 6.0 | ++++ |
| | 12 | 81202 | 4.3 | 1.3 | + |
| | 13 | 188344 | <MIN | 1.6 | − |
| | 14 | 48562 | 3.4 | 1.2 | − |
| | 15 | 39204 | 3.2 | 1.1 | − |
| pLVX MS #5 | 1 | −57642 | <MIN | 1.1 | − |
| 1:25 diluted | 2 | −66350 | <MIN | 1.1 | − |
| Sortgate Low | 3 | 528 | <MIN | 1.0 | − |
| | 4 | −77158 | <MIN | 1.2 | − |
| | 5 | −73694 | <MIN | 0.8 | − |
| | 6 | 2458 | <MIN | 1.2 | − |
| | 7 | −83110 | <MIN | 1.4 | − |
| | 8 | −76824 | <MIN | 1.6 | − |
| | 9 | 35162 | 3.1 | 1.1 | − |
| | 10 | 8447052 | >62.5 | 5.3 | ++++ |
| | 11 | 37314 | 3.1 | 1.5 | − |
| | 12 | 1338 | <MIN | 1.3 | − |
| | 13 | 666 | <MIN | 1.3 | − |
| | 14 | 1728 | <MIN | 1.6 | − |
| | 15 | 414 | <MIN | 1.7 | + |

### EXAMPLE 9

### Infection in the Presence of Two Viruses with Plasmids of Different Antibodies

Cells have been transduced in the presence of a mixture of pLVX mAb1-M and pLVX mAb2-M pool. Cells were transduced with different PCR calculated MOI values (1000, 100, and 40). After transduction single sorted cells clones (IgG+) were stained by incubation with mAb1-antigen-Alexa 488 conjugate and mAb2-antigen-Cy5 conjugate.

The result is shown in FIG. 11.

### EXAMPLE 10

### Infection of Cells with Different Full Length IgG Lentiviral Vectors

Viruses were generated as reported in Example 2 with the following lentiviral expression vectors:
pLVX MS without IRES, two hCMV promoter containing separated expression cassettes for expression of membrane-bound and secreted full length antibody
pLVX MS with IRES, one bicistronic expression cassette with one hCMV promoter for expression of membrane-bound and secreted full length antibody
pLVX M with IRES, one bicistronic expression cassette with one hCMV promoter for expression of membrane-bound full length antibody.

Cells were transduced with diluted viruses comprising these three vectors as described in Example 4. Transduced cells were analyzed by FACS after staining with anti-human IgG (H+L) antibody Alexa 488 conjugate. The results are shown in FIGS. 12 and 13.

### EXAMPLE 11

### Amplification of Nucleic Acids from B-Cells

Total RNA is isolated from antigen-specific B-cells. Single-stranded cDNA is produced with PowerScript™ reverse transcriptase (Clontech) using the template switch protocol (Zhu, et al., BioTechniques 30 (2001) 892-897), with the CDS oligonucleotide (5′-AAG CAG TGG TAA CAA CGC AGA GTA CTT TTT TTT TTT TTT TTT TTT TTT TTT TTT TVN-3′, SEQ ID NO: 36) as primer, and the SMART II oligonucleotide (5′-d[AAG CAG TGG TAA CAA CGC AGA GTA CGC] r[GGG]-3′, SEQ ID NO: 37) as switch template. The cDNA is bulk-amplified by 14 cycles of PCR, using the Advantage2 polymerase mix (Clontech) and an anchor primer (5′-AAG CAG TGG TAT CAA CGC AGA GT-3′, SEQ ID NO: 38) in a total volume of 200 μl. Double-stranded cDNA is purified with the QIAquick PCR purification kit (Qiagen).

Heavy chain variable region coding sequences are amplified with an equimolar mix of one sense primer (SEQ ID NO: 5) plus four antisense primer (SEQ ID NO: 1 to SEQ ID NO: 4) for the hole construct and one sense primer (SEQ ID NO: 11) plus 5 antisense primers (SED ID NO: 6 to SEQ ID NO: 10) for the knob construct; the kappa light chain variable region coding sequences are amplified with an equimolar mix of seven sense primers (SEQ ID NO: 12 to SEQ ID NO: 18) plus an equimolar mix of one antisense primers (SEQ ID NO: 19); and the lambda light chain variable region coding sequences are amplified with an equimolar mix of eight sense primers (SEQ ID NO: 20 to SEQ ID NO: 27) plus an equimolar mix of one antisense primer (SEQ ID NO: 28).

The coding region of the hole and knob heavy chain linked via the IRES can be amplified using the primers SEQ ID NO: 29 and SEQ ID NO: 30.

### EXAMPLE 12

### Enrichment of Cells Displaying Specifically Binding Antibodies by Fluorescence-Activated Cell Sorting

Subconfluent (80%) HEK cells are infected with the full length antibody library or an empty viral vector as a negative control at a multiplicity of infection (MOI) of 0.2. After 5 hours, cells are detached with cell dissociation buffer (Sigma), washed and stained. Half of the cells are stained with Alexa 647 nm-labeled antigen (4 μg/ml) for 30 min. The remaining cells are stained with Alexa 546 nm-labeled antigen (4 μg/ml) and an anti-lentiviral serum from rabbit (diluted 1:6000) for 30 min, followed by staining with Cy5-labeled donkey anti-rabbit IgG (1 μg/m) (Jackson ImmunoResearch Laboratories) for 20 min. All cells are then washed, filtered and stained with propidium iodide (PI) to exclude dead cells. Single cell sorting is performed on a FACS Vantage SE flow cytometer (Becton Dickinson) for, respectively, Alexa 647 nm-positive, PI-negative and, Alexa 546 nm-positive, lentivirus-positive, PI-negative cells.

Each cell is sorted into a well of a 24-well plate containing 50% confluent HEK feeder cells. Upon virus spread (2-3 days post sorting), the infected cells are tested by FACS analysis for antigen binding.

### EXAMPLE 13

### Influence of the Membrane Anchor on Cell Surface Expression of Bispecific Antibodies

1*10⁵ HEK293A cells were seeded in the wells of a 24-well plate and incubated overnight. The next day the cells were co-transfected with 0.5 μg common light chain vector and 0.5 μg of V1.1 to V1.5 shuttle vector driving the expression of two different heavy chains. Heavy chain B binds in combination with the common light chain to an antigen, while the heavy chain N does not bind to the antigen in combination with the common light chain.

Double staining was performed with goat anti human IgG (H+L)—Alexa 488 conjugate (Invitrogen cat. no. A11013) and biotinylated antigen and streptavidine-PE (SA-PE). FACS analysis was performed 48 hours post transfection.

The employed shuttle vectors were:
V.1.1: M-B(knob)-IRES-M-B(hole) (membrane anchor on both binder heavy chains)
V.1.2: M-B(knob)-IRES-M-N(hole) (membrane anchor on both heavy chains, binder and non-binder)
V.1.3: M-N(knob)-IRES-B(hole) (membrane anchor on non-binder only)
V.1.4: B(knob)-IRES-M-N(hole) (membrane anchor on non-binder only)
V.1.5: M-N(knob)-IRES-M-N(hole) (non-binder only, membrane anchor on both heavy chains)
V.1.6: B(hole)-IRES-M-N(knob) (membrane anchor on non-binder, knob and hole exchanged) A transmembrane anchor on the non-binding antibody part (N) is sufficient to display the binding antibody part B on the cell surface when both heavy chain form heterodimers with the knob-into-hole technology (V1.4 FIG. 17) indicating that a single transmembrane anchor is sufficient for the display of a full IgG consisting of two different heavy chains and a common light chain on the cell surface.

1. A method of selecting a cell expressing a bispecific antibody comprising the steps of
(a) generating a population of eukaryotic cells by transduction with a population of lentiviral virus particles, whereby each of the lentiviral virus particles comprises a bicistronic expression cassette, which comprises a first heavy chain variable domain encoding nucleic acid in a hole- or knob-locus upstream of the EV71-IRES and a second heavy chain variable domain encoding nucleic acid in the respective other locus downstream of the EV71-IRES, whereby the first heavy chain variable domain binds to a first antigen and the second variable domain binds to a second antigen, whereby the first antigen and the second antigen can be the same or different, whereby the eukaryotic cell expresses a common light chain, whereby one or both of the heavy chains further comprise a transmembrane domain at their C-terminus, and
(b) selecting from the population of eukaryotic cells a cell depending on the properties of the displayed membrane-bound full length bispecific antibody.

2. The method according to claim 1, characterized in that only the heavy chain downstream of the EV71-IRES comprises a transmembrane domain at its C-terminus.

3. A method of selecting a cell secreting a bispecific antibody comprising the steps of
(a) generating a population of eukaryotic cells by transduction with a population of lentiviral virus particles, whereby each of the lentiviral virus particles comprises a bicistronic expression cassette encoding a secreted bispecific antibody, which comprises a first heavy chain variable domain encoding nucleic acid in a hole- or knob-locus upstream of the EV71-IRES and a second heavy chain variable domain encoding nucleic acid in the respective other locus downstream of the EV71-IRES, whereby the first heavy chain variable domain binds to a first antigen and the second variable domain binds to a second antigen, whereby the first antigen and the second antigen can be the same or different, whereby the eukaryotic cell expresses a common light chain, and
(b) selecting from the population of eukaryotic cells a cell depending on the properties of the secreted full length bispecific antibody.

4. (canceled)

5. The method according to claim 3, characterized in comprising as first step one or more of the following steps:
immunizing a transgenic animal with an antigen of interest, wherein the B-cells of the experimental animal express the same light chain, and/or
selecting the B-cells of the immunized experimental animal by bulk sorting by FACS, and/or
obtaining the heavy chain encoding nucleic acid of each B-cell by individual PCR amplification by two separate/sequential polymerase chain reactions introducing unique restriction sites to enable directed cloning into a shuttle vector/lentiviral expression vector.

6. The method according to claim 5, characterized in comprising the step:
performing a PCR of the complete first heavy chain encoding nucleic acid and the variable domain of the second heavy chain encoding nucleic acid including the EV71-IRES and cloning into second shuttle vector without transmembrane-domain, optionally with the removal of the transmembrane-domain of the first heavy chain if present by restriction cutting and religation of the vector.

7. (canceled)

8. The method according to any one of claims 3, 5 and 6, characterized in that the bicistronic expression cassettes comprises in 5′- to 3′-direction
a promoter,
a first nucleic acid encoding a first full length antibody heavy chain,
the EV71-IRES,
a second nucleic acid encoding a second full length antibody heavy chain.

9. The method according to any one claims 3, 5, 6 and 8, characterized in that the antibody is a bivalent, bispecific antibody.

10. The method according to any one of claims 3, 5, 6, 8 and 9, characterized in that the antibody specifically binds to two different antigens or to two epitopes on the same antigen.

11. The method according to any one of claims 3, 5, 6, 8, 9 and 10, characterized in that the first full length antibody heavy chain comprises a hole mutation and the second antibody heavy chain comprises a knob mutation.

12. (canceled)

13. The method according to any one of claims 3, 5, 6, and 8-11 12, characterized in that the full length antibody comprises a constant region of human origin, especially of human IgG1, IgG2, or IgG4 class.

14. The method according to any one of claims 3, 5, 6, 8-11 and 13, characterized in that the eukaryotic cell is a mammalian cell or a yeast cell.

15. The method according to any one of claims 3, 5, 6, 8-11, 13 and 14, characterized in that the mammalian cell is a CHO cell or a HEK cell.

16. The method according to any one of claims 3, 5, 6, 8-11, 13, 14 and 15, characterized in that the nucleic acid encoding an immunoglobulin heavy chain comprises all exons and all but one intron of the genomically organized immunoglobulin heavy chain gene.

17. (canceled)

18. (canceled)

19. (canceled)

20. The method according to any one of claims 3, 5, 6, 8-11, 13 16, characterized in that the antibody is a humanized or human antibody, especially a human antibody.

21-43. (canceled)

44. A bicistronic expression cassette, which comprises in 5′- to 3′-direction
a promoter,
a first nucleic acid encoding a full length antibody light chain,
the EV71-IRES,
a second nucleic acid encoding a full length antibody heavy chain,
a spliceable intron, and
a nucleic acid encoding a transmembrane domain or a GPI-anchor.

45. A bicistronic expression cassette, which comprises in 5′- to 3′-direction
a promoter,
a first nucleic acid encoding a first full length antibody heavy chain,
the EV71-IRES,
a second nucleic acid encoding a second full length antibody heavy chain, and
a nucleic acid encoding a transmembrane domain or a GPI-anchor

46. The bicistronic expression cassette according to claim 44 or 45, characterized in that the first full length antibody heavy chain comprises a hole mutation and the second antibody heavy chain comprises a knob mutation.

47. The bicistronic expression cassette according to any one of claims 44 to 46, characterized in that the first full length antibody light chain comprises as constant domain a CH1 domain and the first full length antibody heavy chain comprises as first constant domain a CL domain, or the second full length antibody light chain comprises as constant domain a CH1 domain and the second full length antibody heavy chain comprises as first constant domain a CL domain.

48. (canceled)

49. (canceled)

50. The bicistronic expression cassette according to any one of claims 44, 45, 46 and 47, characterized in that the mammalian cell is a CHO cell or a HEK cell.

51. (canceled)

52. (canceled)

53. The bicistronic expression cassette according to any one of claims 44, 45, 46, 47, and 50, characterized in that the transmembrane domain an immunoglobulin transmembrane domain encoded by an M1-M2-exon-fusion of a single exon without the genomically intervening intron.

54. The bicistronic expression cassette according to any one of claims 44, 45, 46, 47, 50 and 53, characterized in that the transmembrane domain is encoded by a cDNA.

55-92. (canceled)

93. A eukaryotic cell library comprising two or more eukaryotic cells each comprising a bicistronic expression cassette according to any one of claims 44, 45, 46, 47, 50, 53 and 54 or a lentiviral vector according to claim 60, wherein the antibodies expressed by each cell differ in at least one amino acid from each other.

94-168. (canceled)

169. A workflow/method for the display of full length bispecific antibodies comprising a common light chain on the surface of eukaryotic cells and the selection of such eukaryotic cells and thereby also the selection of a bispecific antibody comprising the following steps:
a first experimental animal, in one embodiment a transgenic mouse or a transgenic rabbit, is immunized with a first antigen of interest, in one embodiment an extracellular receptor domain, wherein the B-cells of the experimental animal express the same light chain,
a second experimental animal, in one embodiment a transgenic mouse or a transgenic rabbit, is immunized with a second antigen of interest, in one embodiment an extracellular receptor domain, wherein the B-cells of the experimental animal express the same light chain,
whereby the first antigen and the second antigen are different,
selecting the B-cells of the first and second immunized experimental animal, in one embodiment by bulk sorting by FACS,
obtaining the heavy chain encoding nucleic acid of each B-cell by individual PCR amplification by two separate/sequential polymerase chain reactions introducing unique restriction sites to enable directed cloning into a shuttle vector/lentiviral expression vector,
ligation of a first heavy chain variable domain encoding nucleic acid into a shuttle vector/lentiviral expression vector upstream of an IRES, in one embodiment the IRES is the EV71-IRES, into a hole- or knob-locus with transmembrane domain, and ligation of a second heavy chain variable domain encoding nucleic acid into a the same shuttle vector/lentiviral expression vector downstream of the IRES into the respective other locus with a transmembrane domain, i.e. if the heavy chain upstream of the IRES has a hole-locus the heavy chain downstream of the IRES has a knob-locus and vice versa, whereby the first heavy chain variable domain binds to a first antigen and the second variable domain binds to a second antigen, whereby the first antigen and the second antigen are different,
virus generation,
infection of a mammalian cell expressing a common light chain with the virus,
selection of cells displaying bispecific antibodies on their surface by FACS of doubly labeled transduced cells,
PCR of the complete first heavy chain encoding nucleic acid and the variable domain of the second heavy chain encoding nucleic acid (2.2 kbp) including the EV71-IRES and cloning into second shuttle vector without transmembrane-domain; removal of the transmembrane-domain of the first heavy chain by restriction cutting and religation of the vector,
virus generation, infection of a mammalian cell expressing a common light chain, single cell sort of cells, screening of the bispecific antibody in the supernatant, and selection of a bispecific antibody.

170-171. (canceled)

172. A bicistronic expression cassette comprising in 5′- to 3′-direction:
a promoter,
a first nucleic acid encoding a first full length antibody heavy chain,
the EV71-IRES, and
a second nucleic acid encoding a second full length antibody heavy chain.


SEQUENCE LISTING (39 sequences)

SEQ ID NO: 1 - DNA, 35 bp, Artificial Sequence
  PCR primer
tgatatatgctagctgaggagacggtgaccagggt

SEQ ID NO: 2 - DNA, 35 bp, Artificial Sequence
  PCR primer
tgatatatgctagctgaggagacagtgaccagggt

SEQ ID NO: 3 - DNA, 35 bp, Artificial Sequence
  PCR primer
tgatatatgctagctgaagagacggtgaccattgt

SEQ ID NO: 4 - DNA, 35 bp, Artificial Sequence
  PCR primer
tgatatatgctagctgaggagacggtgaccgtggt

SEQ ID NO: 5 - DNA, 40 bp, Artificial Sequence
  PCR primer
ctgttcgggcccacagcgaggtgcagctgktgsagtctgs

SEQ ID NO: 6 - DNA, 36 bp, Artificial Sequence
  PCR primer
agtatatactcgagacggtgaccagggtgccctggc

SEQ ID NO: 7 - DNA, 36 bp, Artificial Sequence
  PCR primer
agtatatactcgagacagtgaccagggtgccacggc

SEQ ID NO: 8 - DNA, 36 bp, Artificial Sequence
  PCR primer
agtatatactcgagacggtgaccattgtcccttggc

SEQ ID NO: 9 - DNA, 36 bp, Artificial Sequence
  PCR primer
agtatatactcgagacggtgaccagggttccctggc

SEQ ID NO: 10 - DNA, 36 bp, Artificial Sequence
  PCR primer
agtatatactcgagacggtgaccgtggtcccttggc

SEQ ID NO: 11 - DNA, 53 bp, Artificial Sequence
  PCR primer
ctggtggcggccgccaccggcgcccacagcgaggtgcagctgktgsagtctgs

SEQ ID NO: 12 - DNA, 42 bp, Artificial Sequence
  PCR primer
gcagccacaggggcccactccgacatccrgwtgacccagtct

SEQ ID NO: 13 - DNA, 42 bp, Artificial Sequence
  PCR primer
gcagccacaggggcccactccgatgttgtgatgactcagtct

SEQ ID NO: 14 - DNA, 42 bp, Artificial Sequence
  PCR primer
gcagccacaggggcccactccgaaattgtgwtgacrcagtct

SEQ ID NO: 15 - DNA, 40 bp, Artificial Sequence
  PCR primer
agctacaggggcccactccgatrttgtgatgacycagwct

SEQ ID NO: 16 - DNA, 39 bp, Artificial Sequence
  PCR primer
gcagccacaggggcccactccgacatcgtgatgacccag

SEQ ID NO: 17 - DNA, 40 bp, Artificial Sequence
  PCR primer
agctacaggggcccactccgaaattgtgctgactcagtct

SEQ ID NO: 18 - DNA, 44 bp, Artificial Sequence
  PCR primer
gcagccacaggggcccactccgawrttgtgmtgackcagtctcc

SEQ ID NO: 19 - DNA, 42 bp, Artificial Sequence
  PCR primer
tgggggcactttctagatcaacactctcccctgttgaagctc

SEQ ID NO: 20 - DNA, 39 bp, Artificial Sequence
  PCR primer
gcagccacaggggcccactcccagtctgtgytgackcag

SEQ ID NO: 21 - DNA, 39 bp, Artificial Sequence
  PCR primer
gcagccacaggggcccactcccagtctgccctgactcag

SEQ ID NO: 22 - DNA, 39 bp, Artificial Sequence
  PCR primer
gcagccacaggggcccactcccagcytgtgctgactcaa

SEQ ID NO: 23 - DNA, 39 bp, Artificial Sequence
  PCR primer
gcagccacaggggcccactcccagsctgtgctgactcag

SEQ ID NO: 24 - DNA, 39 bp, Artificial Sequence
  PCR primer
gcagccacaggggcccactcccagrctgtggtgactcag

SEQ ID NO: 25 - DNA, 39 bp, Artificial Sequence
  PCR primer
gcagccacaggggcccactcccagactgtggtgacccag

SEQ ID NO: 26 - DNA, 39 bp, Artificial Sequence
  PCR primer
gcagccacaggggcccactcccwgcctgtgctgactcag

SEQ ID NO: 27 - DNA, 39 bp, Artificial Sequence
  PCR primer
gcagccacaggggcccactcccaggcagggctgactcag

SEQ ID NO: 28 - DNA, 42 bp, Artificial Sequence
  PCR primer
acattctgtacgtacgactgtcttctccacggtgctcccttc

SEQ ID NO: 29 - DNA, 21 bp, Artificial Sequence
  PCR primer
gatcagcactgaacacagagg

SEQ ID NO: 30 - DNA, 22 bp, Artificial Sequence
  PCr primer
ggaaacacgctagggcctttcg

SEQ ID NO: 31 - DNA, 655 bp, Enterovirus 71
ggcgcgcccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagagcctattgagctagctggtagtcctccggcccctgaatgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaattgtttacctatttattggttttgtaccattatcactgaagtctgtgatcactctcaaattcattttgaccctcaacacaatcaaac

SEQ ID NO: 32 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 33 - DNA, 608 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 34 - DNA, 1517 bp, Artificial Sequence
  intron 6 + M1/M2
gtaaatgagtgcgacggccggcaagcccccgctccccgggctctcgcggtcgcacgaggatgcttggcacgtaccccctgtacatacttcccgggcgcccagcatggaaataaagcacccagcgctgccctggccccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgcgcgtagggacaggccctccctcacccatctacccccacggcactaacccctggcagccctgcccagcctcgcacccacatggggacacaaccgactcctggggacatgcactctcggcccctgtggagggactggtccagatgcccacacacacactcagcccagacccgttcaacaaaccccgcactgaggttggccggccacacggccaccacacacacacgtgcacgcctcacacacggagcttcacccgggcgaaccgcacagcacccagaccagagcaaggtcctcgcacacgtgaacactcctcggacacaggcccccacgagccccacgcggcacctcaaggcccacgagccgctcggcagcttctccacttgctgaccagctcagacaaacccagccctcctctcacaaagtgcccctgcagccgccacacacacacaggcccccacacacaggggaacacacgccacgtcacgtccctggcactggcccacttcccaatacagcccttccctgcagctgaggtcacatgaggtgtgggcttcaccatcctcctgccctctgggcctcagggagggacacgggagacggggagtgggtcctgctgagggccaggcctctatctagggccgggtgtctggctgagtcccggggccaaagctggtgcccagggcgggcagctgtggggagctgacctcaggacactgttggcccatcccggccggcccctacatcctggcccccgccacagagggaatcacccccagaggcccaagcccagggggacacagcactgaccacccccttcctgtccagagctgcaactggaggagagctgtgcggaggcgcaggacggggagctggacgggctgtggacgaccatcaccatcttcatcacactcttcctgctaagcgtgtgctacagtgccaccgtcaccttcttcaaggtgaagtggatcttctcctcggtggtggacctgaagcagaccatcgtccccgactacaggaacatgataaggcagggggcctag

SEQ ID NO: 35 - DNA, 216 bp, Artificial Sequence
  M1/M2
gagctgcaactggaggagagctgtgcggaggcgcaggacggggagctggacgggctgtggacgaccatcaccatcttcatcacactcttcctgctaagcgtgtgctacagtgccaccgtcaccttcttcaaggtgaagtggatcttctcctcggtggtggacctgaagcagaccatcgtccccgactacaggaacatgataaggcagggggcctag

SEQ ID NO: 36 - DNA, 57 bp, Artificial Sequence
  PCR primer
aagcagtggtaacaacgcagagtacttttttttttttttttttttttttttttttvn

SEQ ID NO: 37 - DNA, 30 bp, Artificial Sequence
  PCR primer
aagcagtggtaacaacgcagagtacgcggg

SEQ ID NO: 38 - DNA, 23 bp, Artificial Sequence
  PCR primer
aagcagtggtatcaacgcagagt

SEQ ID NO: 39 - DNA, 20 bp, Artificial Sequence
  PCR primer
ctagaagtcggcggtgtttc